(19) United States
(12) Patent Application Publication
(10) Pub. No.: US 2024/0189410 A1
(43) Pub. Date: Jun. 13, 2024

(54) IMMUNOGENIC COMPOSITIONS AND METHODS FOR ELICITING AN IMMUNE RESPONSE AGAINST CLOSTRIDIOIDES (CLOSTRIDIUM) DIFFICILE

(71) Applicant: Pfizer Inc., New York, NY (US)

(72) Inventors: Annaliesa Sybil Anderson, Upper Saddle River, NJ (US); Marina A. Gonzalez-Garis, Bloomfield, NJ (US); Lei Hu, New City, NY (US); Isis Kanevsky, New York, NY (US); Paul Arthur Liberator, Lewes, DE (US); Justin Keith Moran, West Nyack, NY (US); Lynn Marie Phelan, Lake Hiawatha, NJ (US); Michael William Pride, Staten Island, NY (US); Shuai Shi, Whippany, NJ (US); Naveen Surendran, Dumont, NJ (US)

(21) Appl. No.: 18/535,116

(22) Filed: Dec. 11, 2023

Related U.S. Application Data
(60) Provisional application No. 63/598,556, filed on Nov. 14, 2023.
(60) Provisional application No. 63/485,995, filed on Feb. 20, 2023.
(60) Provisional application No. 63/387,100, filed on Dec. 13, 2022.

(51) Int. Cl. A61K 39/08; A61K 39/39; A61P 31/04

(52) U.S. Cl. CPC A61K 39/08; A61K 39/39; A61P 31/04; A61K 2039/55561

(57) ABSTRACT

The present invention relates to immunogenic compositions that comprise a Clostridioides difficile toxoid A and/or a C. difficile toxoid B, and an adjuvant, and methods of use thereof. The present invention further relate to a methods for eliciting an enhanced immune response in a human against a C. difficile infection. The methods include administering to the human an effective dose of an immunogenic composition, which includes a C. difficile toxoid and an adjuvant, wherein the composition is administered two times.

### REFERENCE TO SEQUENCE LISTING

The instant application contains a sequence listing which has been submitted electronically in .xml format and is hereby incorporated by reference in its entirety. The .xml file, named “PC072922A Sequence Listing.xml”, was created on Nov. 29, 2023, and is 2,360 KB in size.

### FIELD

The present invention is directed to compositions and methods concerning Clostridioides difficile toxoids and methods thereof.

### BACKGROUND

Clostridioides difficile (C. difficile), formerly Clostridium difficile, is a Gram-positive anaerobic bacterium that is associated with gastrointestinal disease in humans. Colonization of C. difficile usually occurs in the colon if the natural gut flora is diminished by treatment with antibiotics. An infection can lead to antibiotic-associated diarrhea and sometimes pseudomembranous colitis through the secretion of the glucosylating toxins, toxin A and toxin B (approximately 308 and 270 kDa, respectively), which are the primary virulence factors of C. difficile.

In the last decade, the numbers and severity of C. difficile outbreaks in hospitals, nursing homes, and other long-term care facilities increased dramatically. Key factors in this escalation include emergence of hypervirulent pathogenic strains, increased use of antibiotics, improved detection methods, and increased exposure to airborne spores in health care facilities.

The increasing burden of C. difficile infection (CDI) on patients and on the healthcare system demonstrates that prevention of CDI constitutes a significant unmet medical need. To date, there is no approved vaccine to prevent primary or recurrent CDI, and treatment options are non-optimal. Thus, there is a need for efficacious C. difficile vaccines for the prevention of CDI.

### SUMMARY OF THE INVENTION

The present invention provides for immunogenic compositions comprising a Clostridioides difficile (C. difficile) toxoid A and/or toxoid B, and an adjuvant. In one aspect, the adjuvant is a CpG adjuvant. In another aspect, the adjuvant is a saponin containing liposomal adjuvant. In one aspect the C. difficile toxoid A comprises the amino acid sequence of SEQ ID NO: 4, wherein the initial methionine is absent. For example, the mutant C. difficile toxin A comprises the amino acid sequence set forth in SEQ ID NO: 84. In one aspect, the C. difficile toxoid B comprises the amino acid sequence of SEQ ID NO: 6, wherein the initial methionine is absent. For example, the mutant C. difficile toxin B comprises the amino acid sequence set forth in SEQ ID NO: 86.

In one aspect, the CpG adjuvant comprises at least on CpG. In one aspect, the immunogenic composition comprises about 0.1 to about 1.0 mg/mL of the CpG. In one aspect, the immunogenic composition comprises 0.5, 1.0 or 3.6 mg/mL of the CpG. In a preferred aspect, the immunogenic composition comprises 3.6 mg/mL of the CpG. In another aspect, the CpG adjuvant further comprises aluminum hydroxide (Al(OH)₃). In one aspect, the immunogenic composition comprises CpG and aluminum hydroxide at about 0.1 to 5 mg/mL or higher. In one aspect, the immunogenic composition comprises CpG and aluminum hydroxide at about 1.0 or 1.5 mg/mL. In a preferred aspect, the immunogenic composition comprises 1.0 mg/mL CpG combined with 1.5 mg/mL aluminum hydroxide. In a preferred aspect, the CpG is CpG 24555.

In one aspect, the CpG adjuvant comprises a histidine or phosphate buffer. In one aspect, the CpG adjuvant comprises sodium chloride.

In another aspect, the adjuvant comprises a saponin containing liposomal adjuvant. In one aspect, the saponin containing liposomal adjuvant comprises a saponin and a monophosphoryl lipid A (MPLA)-containing liposome composition, wherein the liposome composition comprises i) a lipid bilayer comprising phospholipids and ii) cholesterol. In one aspect, the saponin is QS-21. In one aspect, the phospholipids are DMPC and DMPG. In preferred aspect, the saponin containing liposomal adjuvant comprises QS-21, Monophosphoryl Lipid A (MPLA), DMPC, DMPG and cholesterol. In a preferred aspect, the MPLA is Monophosphoryl 3-Deacyl Lipid A.

In one aspect, the immunogenic composition comprises about 0.05 to about 1.0 mg/mL or higher of QS-21. In a preferred aspect, the immunogenic composition comprises 0.2 mg/mL of QS-21. In one aspect, the immunogenic composition comprises about 0.1 to about 1.0 mg/mL or higher of MPLA. In a preferred aspect, the immunogenic composition comprises 0.4 mg/mL of MPLA. In one aspect, the immunogenic composition comprises about 0.5 to about 20 mg/mL or higher of cholesterol. In a preferred aspect, the immunogenic composition comprises 11 mg/mL of cholesterol. In one aspect, the immunogenic composition comprises about 0.5 to about 20 mg/mL or higher of DMPC. In a preferred aspect, the immunogenic composition comprises 14 mg/mL of DMPC. In one aspect, the immunogenic composition comprises about 0.5 to about 3.0 mg/mL or higher of DMPG. In a preferred aspect, the immunogenic composition comprises 1.6 mg/mL of DMPG.

In a preferred aspect, the saponin containing liposomal adjuvant comprises about 0.2 mg/mL QS-21, about 0.4 mg/mL Monophosphoryl 3-Deacyl Lipid A, about 14 mg/mL DMPC, about 1.6 mg/mL DMPG and about 11 mg/mL cholesterol (LiNA-2).

In one aspect, the saponin containing liposomal adjuvant comprises a histidine or phosphate buffer. In one aspect, the saponin containing liposomal adjuvant comprises sodium chloride.

In one aspect, the immunogenic compositions comprise the C. difficile toxoid A and the C. difficile toxoid B in a ratio of about 3:1 to about 1:1. In one aspect, the immunogenic compositions comprise 50-200 μg of toxoid. In one aspect, the immunogenic compositions further comprise at least one of a buffer, a stabilizer, and a surfactant. In one aspect, the immunogenic composition is lyophilized. In one aspect, the lyophilized immunogenic composition is reconstituted prior to administration with the CpG adjuvant or saponin containing liposomal adjuvant.

The present invention further provides for methods of eliciting an immune response in a human subject against Clostridioides difficile, the method comprising administering to a human subject a first dose and a second dose of an immunogenic composition described herein comprising a C. difficile toxoid A and/or C. difficile toxoid B, and a CpG adjuvant or saponin containing liposomal adjuvant.

The present invention further provides for methods of preventing, treating or ameliorating a medically attended C. difficile infection in a human subject, the method comprising administering to a human subject a first dose and a second dose of an immunogenic composition described herein comprising a C. difficile toxoid A and/or C. difficile toxoid B, and a CpG adjuvant or saponin containing liposomal adjuvant.

In one aspect, the method comprises administering an immunogenic composition having a C. difficile toxoid A comprising the amino acid sequence of SEQ ID NO: 4, wherein the initial methionine is absent, and/or a C. difficile toxoid B having the amino acid sequence of SEQ ID NO: 6, wherein the initial methionine is absent. For example, the mutant C. difficile toxin A comprises the amino acid sequence set forth in SEQ ID NO: 84 and/or, the mutant C. difficile toxin B comprises the amino acid sequence set forth in SEQ ID NO: 86.

In one aspect, the second dose is administered about 2 months after the first dose (M 0, 2). In another aspect, the second dose is administered about 6 months after the first dose (M 0, 6).

In one aspect, the method comprises administering an immunogenic composition comprising a CpG adjuvant. In one aspect, the CpG adjuvant is at a dose of 0.5, 1.0 or 3.6 mg/mL. In a preferred aspect, the CpG adjuvant at a dose of 3.6 mg/mL. In another aspect, the method comprises administering an immunogenic composition comprising a CpG adjuvant combined with aluminum hydroxide (Al(OH)₃). In one aspect, the aluminum hydroxide is at a dose of 1.0 or 1.5 mg/mL. In a preferred aspect, the adjuvant comprises CpG at a dose of 1.0 mg/mL combined with 1.5 mg/mL aluminum hydroxide.

In a preferred aspect, the method comprises administering an immunogenic composition comprising CpG 24555. In another preferred aspect, the method comprises administering an immunogenic composition comprising CpG 24555 and aluminum hydroxide. In another preferred aspect, the method comprises administering an immunogenic composition comprising LiNA-2.

In a preferred aspect, the composition is administered at a dose volume of 0.5 mL.

In one aspect, the immune response elicited comprises neutralizing antibodies against C. difficile toxin A and/or neutralizing antibodies against C. difficile toxin B.

In a preferred aspect, the neutralizing antibodies elicited against C. difficile toxin A and/or B is greater after administration of 2 doses of the immunogenic composition compared to administration of 3 doses of the immunogenic composition.

### BRIEF DESCRIPTION OF THE DRAWINGS

FIG. 1 shows the neutralization titers of individual animals (NHPs) immunized with C. difficile toxoid antigens formulated with LiNA-2 adjuvant (2 doses and 3 doses) compared to formulations with aluminum hydroxide (Al(OH)₃) (3 doses). Formulation of toxoid antigens with LiNA-2 elicits a robust and more rapid immune response able to neutralize TcdB cytotoxicity after 2 doses compared to 3 doses formulated with Al(OH)₃.

FIG. 2 shows the neutralization titers of individual animals (NHPs) immunized with C. difficile toxoid antigens formulated with LiNA-2 adjuvant (2 doses—M 0, 2) or CpG adjuvant combined with Al(OH)₃ (2 doses—M 0, 2) compared to formulations with Al(OH)₃ (3 doses). Formulation of toxoid antigens with either LiNA-2 or CpG/Al(OH)₃ elicits robust and more rapid immune responses able to neutralize TcdB cytotoxicity after 2 doses (Month 0, 2) compared to 3 doses (Month 0, 1, 6) formulated with Al(OH)₃.

FIG. 3 shows the neutralization titers of individual animals (NHPs) immunized with C. difficile toxoid antigens formulated with LiNA-2 adjuvant (2 doses—M 0, 6) or CpG adjuvant combined with Al(OH)₃ (2 doses—M 0, 6) compared to formulations with Al(OH)₃ (3 doses). Formulation of toxoid antigens with either LiNA-2 or CpG/Al(OH)₃ elicits robust and rapid immune responses able to neutralize TcdB cytotoxicity after 2 doses (Month 0, 6) compared to 3 doses (Month 0, 1, 6) formulated with Al(OH)₃.

FIG. 4 shows the neutralization titers of individual animals (NHPs) immunized with C. difficile toxoid antigens with two levels of CpG adjuvant (0.5 or 1.0 mg/mL CpG) combined with Al(OH)₃ (2 doses—M 0, 6) compared to formulations with Al(OH)₃ (3 doses). Formulation of toxoid antigens with CpG/Al(OH)₃ elicits robust and rapid immune responses able to neutralize TcdB cytotoxicity after 2 doses (Month 0, 6) compared to 3 doses (Month 0, 1, 6) formulated with Al(OH)₃.

FIG. 5 shows the neutralization titers of individual animals (NHPs) immunized with C. difficile toxoid antigens with CpG adjuvant alone (0.5 or 3.6 mg/mL CpG) or CpG adjuvant combined with Al(OH)₃ at different levels of CpG (0.5, 1 or 3.6 mg/mL CpG) (2 doses—M 0, 2) compared to formulations with Al(OH)₃ (3 doses). Formulation of toxoid antigens with either CpG or CpG/Al(OH)₃ elicits robust and rapid immune responses able to neutralize TcdB cytotoxicity after 2 doses (Month 0, 2) compared to 3 doses (Month 0, 1, 6) formulated with Al(OH)₃.

FIG. 6 shows the neutralization titers of individual animals (rats) immunized with C. difficile toxoid antigens formulated with different LiNA-2 adjuvants (homogeneous and heterogeneous).

FIG. 7 shows the relationship between the % toxoids binding and the mass ratio of the Al(OH)₃/CpG.

### DETAILED DESCRIPTION OF THE INVENTION

The investigational C. difficile vaccine drug product (also referred to as “PF-06425090” or “Al(OH)₃ formulation”, once reconstituted with Al(OH)₃, herein) includes a mixture of genetically modified C. difficile toxoid A (TxdA), i.e., a polypeptide comprising SEQ ID NO: 4, wherein the initial methionine is not present (for example SEQ ID NO: 84) and genetically modified C. difficile toxoid B (TxdB), i.e., a polypeptide comprising SEQ ID NO: 6, wherein the initial methionine is not present (for example SEQ ID NO: 86) that were further chemically inactivated by 1-ethyl-3-(3-dimethylaminopropyl) carbodiimide) (EDC) and N-Hydroxysuccinimide (NHS) to eliminate residual cytotoxicity but retain native antigenic structure and generate a neutralizing antibody response. The investigational C. difficile vaccine is presented as a sterile lyophilized powder with a 1:1 ratio of TxdA and TxdB, in a dosage strength of 200 μg/dose (total dose for TxdA and TxdB) comprising 10 mM Tris buffer at pH 74, 4.5% (w/w) trehalose dihydrate, and 0.01% (w/v) polysorbate 80. Prior to immunization, the lyophilized powder is reconstituted with 1 mg/mL aluminum as aluminum hydroxide (Al(OH)₃) acting as an adsorbent/diluent for a 0.5 mL IM injection. The reconstituted investigational C. difficile vaccine comprises tromethamine, Tris-HCl, trehalose dihydrate, polysorbate 80, sodium chloride and aluminum in the form of aluminum hydroxide. See WIPO Patent Application WO/2012/143902, U.S. Pat. No. 9,187,536, and WIPO Patent Application WO/2014/060898, which are each incorporated by reference herein in their respective entireties.

The investigational C. difficile vaccine was designed to induce protective, functional toxin-neutralizing antibody responses against Toxin A (TcdA) and Toxin B (TcdB) from clinically relevant and hypervirulent C. difficile strains. The vaccine is intended to prevent initial/primary C. difficile infection (CDI) episodes in subjects 50 years of age and older. The vaccine has been shown to be safe and well tolerated after a 3 dose regimen. The investigational C. difficile vaccine has been evaluated in multiple clinical studies, including, B5091001, B5091002, B5091003, B5091007, B5091008, B5091009, B5091010, and B5091019. See also WIPO Patent Application WO/2019/064115, WIPO Patent Application WO/2020/201985, and WIPO Patent Application WO/2021,255690, which are each incorporated by reference herein in their respective entireties.

The investigational C. difficile vaccine reconstituted with Al(OH)₃ is administered at 3 doses schedule (0, 1, 6-month) to elicit a functional immune response required to protect from CDI, in particular, to achieve sufficient toxin-neutralizing titers in subjects with low pre-vaccination toxin B neutralizing titers. Thus, the present invention provides for C. difficile vaccines further comprising an adjuvant to enhance the magnitude, kinetics, and persistence of the anti-toxin immune response to a C. difficile toxoid antigens. The present invention further provides for C. difficile vaccines comprising an adjuvant to enhance the immune response to the C. difficile toxoid antigens, in particular the immune response to toxin B, and potentially reduce the number of doses (2 doses vs 3 doses) required to prevent CDI in humans. Further, a 2 dose schedule would likely increase vaccine adherence and provide an advantage for subjects who have transient risk of CDI. As provided herein, the investigational C. difficile vaccine is formulated with different adjuvants at various doses and compared to the investigational C. difficile vaccine formulated with Al(OH)₃ as an adsorbent.

The present invention provides for the investigational C. difficile vaccine, as described herein, and further comprising an adjuvant. In particular, the present invention provides for the investigational C. difficile vaccine formulated with a CpG adjuvant, a CpG adjuvant in combination with Al(OH)₃ and/or a saponin containing liposomal adjuvant.

In one aspect, the immunogenic compositions of the present invention comprise genetically modified C. difficile toxoid A (TxdA), i.e., a polypeptide comprising SEQ ID NO: 4, wherein the initial methionine is not present (for example SEQ ID NO: 84) and genetically modified C. difficile toxoid B (TxdB), i.e., a polypeptide comprising SEQ ID NO: 6, wherein the initial methionine is not present (for example SEQ ID NO: 86) at 200 μg/dose (total dose for TxdA and TxdB) with a 1:1 ratio of TxdA and TxdB, and an adjuvant.

In one aspect, the adjuvant is a CpG adjuvant, which comprises at least one CpG oligonucleotide, preferably a CpG oligodeoxynucleotides (ODN). In one aspect the CpG is a B class CpG. In one aspect the CpG is CpG 24555, a 21-mer oligodeoxynucleotide TLR9 agonist. In one aspect, the adjuvant comprises CpG alone. In one aspect, the CpG is combined with aluminum hydroxide (Al(OH)₃). In one aspect, the CpG adjuvant comprising CpG 24555 alone, or combined with aluminum hydroxide (Al(OH)₃), is designed to reconstitute lyophilized the investigational C. difficile vaccine drug product for administration.

In one aspect, the immunogenic composition comprises about 0.1 to about 5 mg/mL CpG. In one aspect, the immunogenic composition comprises about 0.5, about 1.0 or about 3.6 mg/mL CpG. In one aspect, the immunogenic composition comprises 0.5 mg/mL CpG alone, or combined with aluminum hydroxide (AlOH₃). In one aspect, the immunogenic composition comprises 0.5 mg/mL CpG alone. In one aspect, the immunogenic composition comprises 0.5 mg/mL CpG combined with 1.0 mg/mL AlOH₃. In one aspect, the immunogenic composition comprises 0.5 mg/mL CpG combined with 1.5 mg/mL AlOH₃.

In one aspect, the immunogenic composition comprises 1.0 mg/mL CpG alone, or combined with aluminum hydroxide (AlOH₃). In one aspect, the immunogenic composition comprises 1.0 mg/mL CpG alone. In one aspect, the immunogenic composition comprises 1.0 mg/mL CpG combined with 1.0 mg/mL AlOH₃. In one aspect, the immunogenic composition comprises 1.0 mg/mL CpG combined with 1.5 mg/mL AlOH₃.

In one aspect, the immunogenic composition comprises 3.6 mg/mL CpG alone, or combined with aluminum hydroxide (AlOH₃). In one aspect, the immunogenic composition comprises 3.6 mg/mL CpG alone. In one aspect, the immunogenic composition comprises 3.6 mg/mL CpG combined with 1.0 mg/mL AlOH₃. In one aspect, the immunogenic composition comprises 3.6 mg/mL CpG combined with 1.5 mg/mL AlOH₃. In one aspect, the immunogenic composition comprises 3.6 mg/mL CpG combined with 1.8 mg/mL aluminum hydroxide AlOH₃.

In one aspect, the immunogenic composition comprises a CpG combined with about 1.0, about 1.5, about 1.7, about 1.8, about 2.0, or about 2.5 mg/mL Al(OH)₃.

In a preferred aspect, the CpG adjuvant comprises CpG alone. In a preferred aspect, the CpG adjuvant comprises CpG in a histidine buffer and sodium chloride (NaCl) at a pH of about 6.5. In a preferred aspect, the CpG adjuvant comprises CpG in 10 mM histidine buffer and 60 mM sodium chloride (NaCl) at a pH of about 6.5.

In a preferred aspect, the CpG adjuvant comprises 3.6 mg/mL CpG 24555 (i.e. high-dose CpG). In a preferred aspect, the CpG adjuvant comprises 3.6 mg/mL CpG 24555 in a histidine buffer and sodium chloride (NaCl) at a pH of about 6.5. In a preferred aspect, the CpG adjuvant comprises 3.6 mg/mL CpG 24555 in 10 mM histidine buffer and 60 mM sodium chloride (NaCl) at a pH of about 6.5.

In another preferred aspect, the CpG adjuvant comprises 1.0 mg/mL CpG 24555 combined with 1.5 mg/mL AlOH₃ (i.e. low-dose CpG). In another preferred aspect, the CpG adjuvant comprises 1.0 mg/mL CpG 24555 combined with 1.5 mg/mL AlOH₃ in a histidine buffer and sodium chloride (NaCl) at a pH of about 6.5. In another preferred aspect, the CpG adjuvant comprises 1.0 mg/mL CpG combined with 1.5 mg/mL AlOH₃ in 10 mM histidine buffer and 50 mM sodium chloride (NaCl) at a pH of about 6.5.

In another preferred aspect, the CpG adjuvant comprises CpG combined with AlOH₃ in a histidine buffer and sodium chloride (NaCl) at a pH of about 6.5. In another preferred aspect, the CpG adjuvant comprises CpG combined with AlOH₃ in 10 mM histidine buffer and 50 mM sodium chloride (NaCl) at a pH of about 6.5.

The present invention provides for immunogenic compositions comprising genetically modified C. difficile toxoid A (TxdA), i.e., a polypeptide comprising SEQ ID NO: 4, wherein the initial methionine is not present (for example SEQ ID NO: 84) and genetically modified C. difficile toxoid B (TxdB), i.e., a polypeptide comprising SEQ ID NO: 6, wherein the initial methionine is not present (for example SEQ ID NO: 86) at 200 μg/dose (total dose for TxdA and TxdB) and a CpG adjuvant comprising CpG 24555. In one aspect, the immunogenic composition comprises 0.5, 1.0, or 3.6 mg/mL CpG 24555. In a preferred aspect, the immunogenic composition comprises 3.6 mg/mL CpG 24555. In a preferred aspect, the immunogenic composition comprises 3.6 mg/mL CpG 24555 in a histidine buffer and sodium chloride (NaCl). In a preferred aspect, the immunogenic composition comprises 3.6 mg/mL CpG 24555 in 10 mM histidine buffer and 60 mM sodium chloride (NaCl).

In a preferred aspect, the immunogenic composition comprises the investigational C. difficile vaccine and 3.6 mg/mL CpG 24555. In a preferred aspect, the lyophilized investigational C. difficile vaccine is reconstituted with 3.6 mg/mL CpG 24555 in a histidine buffer and sodium chloride (NaCl). In a preferred aspect, the lyophilized investigational C. difficile vaccine is reconstituted with 3.6 mg/mL CpG 24555 in a 10 mM histidine buffer and 60 mM sodium chloride (NaCl).

The present invention further provides for immunogenic compositions comprising genetically modified C. difficile toxoid A (TxdA), i.e., a polypeptide comprising SEQ ID NO: 4, wherein the initial methionine is not present (for example SEQ ID NO: 84) and genetically modified C. difficile toxoid B (TxdB), i.e., a polypeptide comprising SEQ ID NO: 6, wherein the initial methionine is not present (for example SEQ ID NO: 86) at 200 μg/dose (total dose for TxdA and TxdB), a CpG adjuvant comprising CpG 24555 and aluminum hydroxide Al(OH)₃. In one aspect, the immunogenic composition comprises 0.5, 1.0, or 3.6 mg/mL CpG 24555 and aluminum hydroxide Al(OH)₃. In one aspect, the immunogenic composition comprises CpG 24555 and 1.0 or 1.5 mg/mL aluminum hydroxide Al(OH)₃. In a preferred aspect, the immunogenic composition comprises 1.0 mg/mL CpG 24555 and 1.5 mg/mL aluminum hydroxide Al(OH)₃. In a preferred aspect, the immunogenic composition comprises 1.0 mg/mL CpG 24555 and 1.5 mg/mL AlOH₃ in a histidine buffer and sodium chloride (NaCl). In a preferred aspect, the immunogenic composition comprises 1.0 mg/mL CpG 24555 and 1.5 mg/mL AlOH₃ in 10 mM histidine buffer and 50 mM sodium chloride (NaCl).

In a preferred aspect, the immunogenic composition comprises the investigational C. difficile vaccine, 1.0 mg/mL CpG 24555 and 1.5 mg/mL aluminum hydroxide Al(OH)₃. In a preferred aspect, the lyophilized investigational C. difficile vaccine is reconstituted with 1.0 mg/mL CpG 24555 and 1.5 mg/mL aluminum hydroxide Al(OH)₃ in a histidine buffer comprising sodium chloride (NaCl). In a preferred aspect, the lyophilized investigational C. difficile vaccine is reconstituted with 1.0 mg/mL CpG 24555 and 1.5 mg/mL aluminum hydroxide Al(OH)₃ in 10 mM histidine buffer and 50 mM sodium chloride (NaCl).

In one aspect, an adjuvanted immunogenic composition of the present invention comprises a C. difficile toxoid A (TxdA) comprising SEQ ID NO: 4, wherein the initial methionine is not present (for example SEQ ID NO: 84), and a C. difficile toxoid B (TxdB) comprising SEQ ID NO: 6, wherein the initial methionine is not present (for example SEQ ID NO: 86), and a CpG (e.g. CpG 24555) adjuvant alone or in combination with aluminum (e.g. aluminum hydroxide Al(OH)₃).

In another aspect, the adjuvant is a saponin containing liposomal adjuvant, which comprises a monophosphoryl lipid A (MPLA) and a triterpenoid glycoside saponin (QS-21). In one aspect, the saponin containing liposomal adjuvant further comprises 1,2-dimyristoyl-sn-glycero-3-phosphocholine (DMPC). In one aspect, the saponin containing liposomal adjuvant further comprises 1,2-dimyristoyl-sn-glycero-3-phospho-(1′-rac-glycerol) (DMPG). In one aspect, the saponin containing liposomal adjuvant further comprises cholesterol.

In a preferred aspect, the saponin containing liposomal adjuvant comprises MPLA (e.g. 3D-PHAD®), QS-21, DMPC, DMPG and cholesterol. In a preferred aspect, the saponin containing liposomal adjuvant comprises 0.4 mg/mL MPLA (e.g. 3D-PHAD®), 0.2 mg/mL QS-21, 14 mg/mL DMPC, 1.6 mg/mL DMPG and 11/mg/mL cholesterol (i.e. LiNA-2). In another preferred aspect, the saponin containing liposomal adjuvant comprises MPLA (e.g. 3D-PHAD®), QS-21, DMPC, DMPG and cholesterol in a phosphate buffer and sodium chloride (NaCl). In another preferred aspect, the saponin containing liposomal adjuvant comprises MPLA (e.g. 3D-PHAD®), QS-21, DMPC, DMPG and cholesterol in 10 mM phosphate buffer and 150 mM sodium chloride (NaCl). In another preferred aspect, the saponin containing liposomal adjuvant comprises MPLA (e.g. 3D-PHAD®), QS-21, DMPC, DMPG and cholesterol in a phosphate buffer and sodium chloride (NaCl) at a pH of about 6.2. In a preferred aspect, the saponin containing liposomal adjuvant is designed to reconstitute the lyophilized investigational C. difficile vaccine drug product for administration.

The present invention further provides for immunogenic compositions comprising genetically modified C. difficile toxoid A (TxdA), i.e., a polypeptide comprising SEQ ID NO: 4, wherein the initial methionine is not present (for example SEQ ID NO: 84) and genetically modified C. difficile toxoid B (TxdB), i.e., a polypeptide comprising SEQ ID NO: 6, wherein the initial methionine is not present (for example SEQ ID NO: 86) at 200 μg/dose (total dose for TxdA and TxdB) and a saponin containing liposomal adjuvant (e.g. LiNA-2). In one aspect, the immunogenic composition comprises a saponin containing liposomal adjuvant comprising MPLA (e.g. 3D-PHAD®) and QS-21. In a preferred aspect, the immunogenic composition comprises a saponin containing liposomal adjuvant comprising MPLA (e.g. 3D-PHAD®), QS-21, DMPC, DMPG and cholesterol in a phosphate buffer and sodium chloride (NaCl). In a preferred aspect, the immunogenic composition comprises a saponin containing liposomal adjuvant comprising MPLA (e.g. 3D-PHAD®), QS-21, DMPC, DMPG and cholesterol in a 10 mM phosphate buffer and 150 mM sodium chloride (NaCl). In a preferred aspect, the immunogenic composition comprises 0.4 mg/mL MPLA (e.g. 3D-PHAD®), 0.2 mg/mL QS-21, 14 mg/mL DMPC, 1.6 mg/mL DMPG and 11/mg/mL cholesterol. In a preferred aspect, the immunogenic composition comprises 0.4 mg/mL MPLA (e.g. 3D-PHAD®), 0.2 mg/mL QS-21, 14 mg/mL DMPC, 1.6 mg/mL DMPG and 11/mg/mL cholesterol in a 10 mM phosphate buffer and 150 mM sodium chloride (NaCl).

In a preferred aspect, the immunogenic composition comprises the investigational C. difficile vaccine, 0.4 mg/mL MPLA (e.g. 3D-PHAD®), 0.2 mg/mL QS-21, 14 mg/mL DMPC, 1.6 mg/mL DMPG and 11/mg/mL cholesterol. In a preferred aspect, the lyophilized investigational C. difficile vaccine is reconstituted with 0.4 mg/mL MPLA (e.g. 3D-PHAD®), 0.2 mg/mL QS-21, 14 mg/mL DMPC, 1.6 mg/mL DMPG and 11/mg/mL cholesterol in a phosphate buffer and sodium chloride (NaCl). In a preferred aspect, the lyophilized investigational C. difficile vaccine is reconstituted with 0.4 mg/mL MPLA (e.g. 3D-PHAD®), 0.2 mg/mL QS-21, 14 mg/mL DMPC, 1.6 mg/mL DMPG and 11/mg/mL in a 10 mM phosphate buffer and 150 mM sodium chloride (NaCl). In a preferred aspect, the immunogenic composition comprises the investigational C. difficile vaccine and LiNA-2. In a preferred aspect, the lyophilized investigational C. difficile vaccine is reconstituted with LiNA-2 in a phosphate buffer comprising sodium chloride (NaCl). In a preferred aspect, the lyophilized investigational C. difficile vaccine is reconstituted with LiNA-2 in 10 mM phosphate buffer and 150 mM sodium chloride (NaCl).

In one aspect, an adjuvanted immunogenic composition of the present invention comprises a C. difficile toxoid A (TxdA) comprising SEQ ID NO: 4, wherein the initial methionine is not present (for example SEQ ID NO: 84), and a C. difficile toxoid B (TxdB) comprising SEQ ID NO: 6, wherein the initial methionine is not present (for example SEQ ID NO: 86), and a saponin containing liposomal adjuvant (e.g. LiNA-2).

In one aspect, immunogenic compositions of the present invention are prepared by reconstituting the lyophilized investigational C. difficile vaccine for injection with either CpG (e.g. CpG 24555), CpG (e.g. CpG 24555) combined with aluminum hydroxide (AlOH₃), or a saponin containing liposomal adjuvant (e.g. LiNA-2). The reconstituted immunogenic compositions are withdrawn in the syringe using the vial adapter to enable a dose of 0.5 mL for IM administration.

In one aspect, immunogenic compositions of the present invention are prepared by reconstituting the lyophilized investigational C. difficile vaccine for injection with aluminum hydroxide (AlOH₃) adsorbent (current formulation). The reconstituted immunogenic compositions are presented as a sterile liquid suspension in a dosage strength of 1 mg/mL aluminum in the form of aluminum hydroxide containing sodium chloride (NaCl).

In one aspect, the immunogenic compositions disclosed herein are administered two times each dose being separated from one another by about one to about four months. In one aspect, the immunogenic compositions disclosed herein are administered two times each dose being separated from one another by about one, two, three or four months. In a preferred aspect, the immunogenic compositions disclosed herein are administered two times each dose being separated from one another by about two months. In one aspect, methods of the present invention include administering a first dose and a second dose of a immunogenic composition described herein, preferably the second dose is administered about 2 months after the first dose (M 0, 2).

In another aspect, the immunogenic compositions disclosed herein are administered two times each dose being separated from one another by about one to about eight months. In one aspect, the immunogenic compositions disclosed herein are administered two times each dose being separated from one another by about one, two, three, four, five, six, seven or eight months. In a preferred aspect, the immunogenic compositions disclosed herein are administered two times each dose being separated from one another by about six months. In one aspect, methods of the present invention include administering a first dose and a second dose of a immunogenic composition described herein, preferably the second dose is administered about 6 months after the first dose (M 0, 6).

In one aspect, the method includes further administration of a booster dose after the second dose, e.g. 6 months or 12 months after the second dose. Further boosters may be administered. In one aspect, the method does not include further administration of a booster after the second dose.

In one aspect, the immunogenic compositions disclosed herein are administered three times the first and second dose being separated from one another by about one to about four months and the third dose being separated from the first dose by about 5 to 10 months. In a preferred aspect, the immunogenic compositions disclosed herein are administered three times the first and second dose being separated from one another by about one month and the third dose being separated from the first dose by about 6 months. In one aspect, methods of the present invention include administering a first dose, a second dose and a third dose of a immunogenic composition described herein, preferably the second dose is administered about 1 month after the first dose and the third dose is administered about 6 months after the first dose (M 0, 1, 6).

In one aspect, the method includes further administration of a booster dose after the third dose, e.g. 6 months or 12 months after the third dose. Further boosters may be administered. In one aspect, the method does not include further administration of a booster after the third dose.

Examples 1 to 4 of the present invention compare the relative immunogenicity in nonhuman primates (NHPs) of C. difficile toxoid antigens formulated with adjuvant formulations compared to an Al(OH)₃ formulation. The formulations were assessed for enhanced immune responses, in particular the immune response to toxin B, and potential reduction in the number of doses (2 doses vs 3 doses) required to prevent C. difficile infection (CDI) in humans.

As provided in the examples herein, multiple immunogenicity studies were conducted in NHPs to compare the functional response of adjuvanted formulations to Al(OH)₃ formulated toxoid antigens. See Examples 1-4. The adjuvanted formulations including a CpG adjuvant (e.g. CpG 24555), both with and without Al(OH)₃, as well as a saponin containing liposomal adjuvant (e.g. LiNA-2), elicited a robust and more rapid immune response in NHPs following 2 doses (at Month 0, 2 or Month 0, 6) compared to 3 doses (at Months 0, 1, and 6) of the Al(OH)₃ formulation.

As further provided in the examples herein, dose-ranging immunogenicity studies were conducted with CpG-adjuvanted C. difficile toxoid antigens in NHPs. The functional immune response to 2 doses (at Months 0, 2) of toxoid antigen increased in parallel with the amount of CpG included (0.5, 1.0, and 3.6 mg/mL CpG 24555) when formulated together with Al(OH)₃. The inclusion of CpG at either 1.0 or 3.6 mg/mL elicited a more rapid and robust immune response than 3 doses of the Al(OH)₃ formulation. See Examples 3 and 4. The functional immune response to 2 doses (at Months 0, 2) of toxoid antigen formulated with either 0.5 or 3.6 mg/mL CpG alone (no Al(OH)₃) was also evaluated in NHPs. Both elicited a more rapid response compared to 3 doses of the Al(OH)₃ formulation. See Example 4.

Example 5 provides immunogenicity studies conducted with homogeneous and heterogenous LiNA-2-adjuvanted C. difficile toxoid antigens in rats. The toxoids formulated with homogeneous and heterogeneous saponin containing liposomal adjuvant (e.g. LiNA-2) elicited similar immune responses able to neutralize Toxin B cytotoxicity.

Example 6 of the present invention provides binding studies with various CpG and Al(OH)₃ concentrations and ratios to understand the binding properties of the C. difficile toxoids and CpG adjuvant.

Example 7 of the present invention provides binding and resuspension studies for various CpG/Al(OH)₃ formulations to assess the properties of the C. difficile toxoids and CpG adjuvant.

Example 8 provides preferred immunogenic compositions and administration regimens of the present invention. For example, immunogenic compositions formulated with CpG adjuvant alone, CpG adjuvant+Al(OH)₃ and/or a saponin containing liposomal adjuvant (LiNA-2) administered in 2 doses (e.g. M 0, 2 or M 0, 6).

### Composition and Vaccine

In one aspect, the composition is an immunogenic composition. In one aspect, the composition is an immunogenic composition for a human. In another aspect, the composition is a vaccine. A “vaccine” refers to a composition that includes an antigen, which contains at least one epitope that induces an immune response that is specific for that antigen. The vaccine may be administered directly into the subject by subcutaneous, oral, oronasal, or intranasal routes of administration. Preferably, the vaccine is administered intramuscularly. In one aspect, the composition is a human vaccine. In one aspect, the composition is an immunogenic composition against C. difficile. In certain aspects, the compositions may further comprise one or more C. difficile antigens, one or more pharmaceutically acceptable carriers and/or one or more adjuvants, as described herein.

As described above, the investigational C. difficile vaccine (also referred to as “PF-06425090” or “Al(OH)₃ formulation”, once reconstituted with Al(OH)₃, herein) includes a mixture of genetically modified C. difficile toxoid A (TxdA), i.e., a polypeptide comprising SEQ ID NO: 4, wherein the initial methionine is not present (for example SEQ ID NO: 84) and genetically modified C. difficile toxoid B (TxdB), i.e., a polypeptide comprising SEQ ID NO: 6, wherein the initial methionine is not present (for example SEQ ID NO: 86) that were further chemically inactivated by 1-ethyl-3-(3-dimethylaminopropyl) carbodiimide) (EDC) and N-Hydroxysuccinimide (NHS). The vaccine is presented as a lyophilized powder with a 1:1 ratio of TxdA and TxdB in a dosage strength of 200 μg/dose (total dose for TxdA and TxdB). Prior to immunization, the investigational C. difficile vaccine is reconstituted with Al(OH)₃ as an adsorbent. The vaccine is administered in 3 doses (M 0, 1, 6).

The present invention provides for the investigational C. difficile vaccine formulated to include an oligonucleotide CpG adjuvant (e.g. CpG 24555), a CpG adjuvant (e.g. CpG 24555) combined with Al(OH)₃ and/or a saponin containing liposomal adjuvant (e.g. LiNA-2) to enhance the immune response, in particular the immune response to toxin B, thus providing a more rapid onset of protection and use of a 2-dose regimen (M 0, 2 or Month 0, 6). The adjuvanted vaccine compositions described herein are presented as a lyophilized dosage form (investigational C. difficile vaccine) and prepared for injection by reconstituting with either an oligonucleotide CpG adjuvant (e.g. CpG 24555), an oligonucleotide CpG adjuvant (e.g. CpG 24555) combined with aluminum hydroxide Al(OH)₃, or a saponin containing liposomal adjuvant (e.g. LiNA-2).

C. difficile Toxoids

The term “C. difficile toxoid” is used herein to refer to a C. difficile toxin (Toxin A or Toxin B) that has been partially or completely inactivated. A toxin is inactivated if it has less toxicity (e.g., 100%, 99%, 98%, 80%, 70%, 60%, 50%, 40%, 30%, 20%, 10% or less toxicity or any value therebetween) than untreated toxin, as measured by for example an in vitro cytotoxicity assay or by animal toxicity. C. difficile toxoids can be produced by purification of toxins from C. difficile cultures and inactivation of toxins by chemical (e.g., formaldehyde, glutaraldehyde, peroxide or oxygen treatment). Alternatively, wild type or mutant C. difficile toxins that lack or have reduced toxicity can be produced using recombinant methods and/or alternative chemical crosslinking agents. For example, genetic mutations resulting in reduced toxicity can be made. Wild type or mutant C. difficile toxins lacking specific regions to reduce toxicity can also be made.

The C. difficile toxoid or mutant C. difficile toxin refers to a molecule that exhibits a structure or sequence that differs from the corresponding wild-type structure or sequence, e.g., by having crosslinks as compared to the corresponding wild-type structure and/or by having at least one mutation, as compared to the corresponding wild-type sequence when optimally aligned, such as by the programs GAP or BESTFIT using default gap weights. The term toxoid or mutant toxin as used herein further exhibits a functional property (e.g., abrogated glucosyltransferase and/or abrogated cysteine protease activity) that differs from the corresponding wild-type molecule.

The toxoid as used herein may be any of the toxoids or mutant C. difficile toxins as described in WIPO Patent Application WO/2012/143902, U.S. Pat. No. 9,187,536, and WIPO Patent Application WO/2014/060898, which are each incorporated by reference herein in their respective entireties. That is, the toxoid as used herein may be any of the polypeptides as described in WIPO Patent Application WO/2012/143902, U.S. Pat. No. 9,187,536, and WIPO Patent Application WO/2014/060898, which are each incorporated by reference herein in their respective entireties. A C. difficile toxin from any of the wild-type strains described above may be used as a source from which a toxoid or mutant C. difficile toxin is produced. Preferably, C. difficile 630 is the source from which a C. difficile toxoid is produced.

In one aspect, the toxoid refers to a polypeptide that has any one sequence selected from the toxoid polypeptides of SEQ ID NO: 1 to SEQ ID NO: 861, wherein the initial methionine is absent, and wherein the polypeptide has been contacted with a chemical crosslinker, such as, for example, formaldehyde or EDC, as described herein, and/or has been genetically mutated. More specifically, in one aspect, the toxoid is a polypeptide having the amino acid sequence set forth in any one of SEQ ID NOs: 1-8, 15, 17, 19, 21, 23, 25, 28-35, 82-761, and 762-840. In another aspect, the polypeptide has an amino acid sequence that is about 60%, 65%, 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOs: 1-8, 15, 17, 19, 21, 23, 25, 28-35, 82-761, and 762-840. In another aspect, the polypeptide has an amino acid sequence having at least 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, or 2200 consecutive amino acids to any one of SEQ ID NOs: 1-8, 15, 17, 19, 21, 23, 25, 28-35, 82-761, and 762-840.

The mutation may involve a substitution, deletion, truncation or modification of the wild type amino acid residue normally located at that position. Accordingly, the polypeptide may be any one of a fusion polypeptide, glycosylated polypeptide, non-glycosylated polypeptide, lipidated polypeptide, non-lipidated polypeptide, phosphorylated polypeptide, non-phosphorylated polypeptide, myristoylated polypeptide, non-myristoylated polypeptide, monomeric polypeptide, multimeric polypeptide, particulate polypeptide, denatured polypeptide, etc. Preferably, the mutation is a non-conservative amino acid substitution. The mutant toxins of the invention may be prepared by techniques known in the art for preparing mutations, such as, for example, site-directed mutagenesis, mutagenesis using a mutagen (e.g., UV light), etc. Preferably, site-directed mutagenesis is used. Alternatively, a nucleic acid molecule having an objective sequence may be directly synthesized. Such chemical synthesis methods are known in the art.

In the present invention, the mutant C. difficile toxin includes at least one mutation in a glucosyltransferase domain, relative to the corresponding wild-type C. difficile toxin. In one aspect, the glucosyltransferase domain includes at least two mutations. Preferably, the mutation decreases or abrogates glucosyltransferase enzyme activity of the toxin, as compared to the glucosyltransferase enzyme activity of the corresponding wild-type C. difficile toxin.

An exemplary C. difficile toxoid A includes the amino acid sequence set forth in SEQ ID NO: 4, wherein the initial methionine is not present. SEQ ID NO: 4 has D285A, D287A and C700A mutations, as compared to SEQ ID NO: 1 (wild-type toxin A). In another aspect, the mutant C. difficile toxin A includes the amino acid sequence set forth in SEQ ID NO: 84. In another aspect, the C. difficile toxoid A includes a glucosyltransferase domain including SEQ ID NO: 29 having an amino acid substitution at positions 285 and 287, and a cysteine protease domain comprising SEQ ID NO: 32 having an amino acid substitution at position 158, relative to the corresponding wild-type C. difficile toxin A. In another aspect, the C. difficile toxoid A includes the amino acid sequence set forth in SEQ ID NO: 1, wherein the initial methionine is not present, having D285A, D287A, and C700A mutations.

Further examples of a C. difficile toxoid A include the amino acid sequence set forth in SEQ ID NO: 7, which has a D269A, R272A, D285A, D287A, E460A, R462A, and C700A mutation, as compared to SEQ ID NO: 1, wherein the initial methionine is optionally not present. In another aspect, the mutant C. difficile toxin A includes the amino acid sequence set forth in SEQ ID NO: 83.

An exemplary C. difficile toxoid B includes the amino acid sequence set forth in SEQ ID NO: 6, wherein the initial methionine is not present. SEQ ID NO: 6 has D286A, D288A and C689A mutations, as compared to SEQ ID NO: 2 (wild-type toxin B). In another aspect, the mutant C. difficile toxin A includes the amino acid sequence set forth in SEQ ID NO: 86.

Further examples of a mutant C. difficile TcdB include the amino acid sequence set forth in SEQ ID NO: 8, which has a D270A, R273A, D286A, D288A, D461A, K463A, and C698A mutation, as compared to SEQ ID NO: 2, and wherein the initial methionine of SEQ ID NO: 8 is optionally not present. In another aspect, the mutant C. difficile toxin B includes the amino acid sequence set forth in SEQ ID NO: 85. In another aspect, the C. difficile toxoid B includes the amino acid sequence set forth in SEQ ID NO: 2, wherein the initial methionine is not present, having D286A, D288A and C689A mutations.

In addition to generating an immune response in a mammal, the toxoids described herein also have reduced cytotoxicity compared to the corresponding wild-type C. difficile toxin. Preferably, the immunogenic compositions are safe and have minimal (e.g., about a 6-8 log₁₀ reduction) to no cytotoxicity, relative to the cytotoxicity of a respective wild-type toxin, for administration in mammals.

As used herein, the term cytotoxicity is a term understood in the art and refers to apoptotic cell death and/or a state in which one or more usual biochemical or biological functions of a cell are aberrantly compromised, as compared to an identical cell under identical conditions but in the absence of the cytotoxic agent. Toxicity can be quantitated, for example, in cells or in mammals as the amount of an agent needed to induce 50% cell death (i.e., EC₅₀ or ED₅₀, respectively) or by other methods known in the art.

In one aspect, the toxoid is a polypeptide that has any one sequence selected from the toxoid polypeptides of SEQ ID NO: 1 to SEQ ID NO: 861, more specifically, the toxoid is a polypeptide having the amino acid sequence set forth in any one of SEQ ID NOs: 1-8, 15, 17, 19, 21, 23, 25, 28-35, 82-761, and 762-840, wherein the initial methionine is absent, and wherein the polypeptide has been contacted with a chemical crosslinker, such as, for example, formaldehyde or EDC, as described herein. Crosslinking (also referred to as “chemical inactivation” or “inactivation” herein) is a process of chemically joining two or more molecules by a covalent bond. The terms “crosslinking reagents,” “crosslinking agents,” and “crosslinkers” refer to molecules that are capable of reacting with and/or chemically attaching to specific functional groups (primary amines, sulfhydryls, carboxyls, carbonyls, etc.) on peptides, polypeptides, and/or proteins. In one aspect, the molecule may contain two or more reactive ends that are capable of reacting with and/or chemically attaching to specific functional groups (primary amines, sulfhydryls, carboxyls, carbonyls, etc.) on peptides, polypeptides, and/or proteins. Preferably, the chemical crosslinking agent is water-soluble. In another preferred aspect, the chemical crosslinking agent is a heterobifunctional crosslinker. In another aspect, the chemical crosslinking agent is not a bifunctional crosslinker. Chemical crosslinking agents are known in the art.

Exemplary suitable chemical crosslinking agents include formaldehyde; formalin; acetaldehyde; propionaldehyde; water-soluble carbodiimides (RN═C═NR′), which include 1-Ethyl-3-(3-Dimethylaminopropyl)-Carbodiimide (EDC), 1-Ethyl-3-(3-Dimethylaminopropyl)-Carbodiimide Hydrochloride, 1-Cyclohexyl-3-(2-morpholinyl-(4-ethyl)carbodiimide metho-p-toluenesulfonate (CMC), N,N′-dicyclohexylcarbodiimide (DCC), and N,N′-diisopropylcarbodiimide (DIC), and derivatives thereof; and N-hydroxysuccinimide (NHS); phenylglyoxal; and/or UDP-dialdehyde.

In another aspect, the at least one amino acid may be chemically crosslinked by an agent that includes EDC and NHS. For example, in one aspect, the invention relates to an isolated polypeptide having the amino acid sequence set forth in SEQ ID NO: 4, wherein the methionine residue at position 1 is optionally not present, wherein the polypeptide includes at least one amino acid side chain chemically modified by EDC and NHS. In another aspect, the invention relates to an isolated polypeptide having the amino acid sequence set forth in SEQ ID NO: 6, wherein the methionine residue at position 1 is optionally not present, wherein the polypeptide includes at least one amino acid side chain chemically modified by EDC and NHS. In yet another aspect, the invention relates to an isolated polypeptide having the amino acid sequence set forth in SEQ ID NO: 84, SEQ ID NO: 86, SEQ ID NO: 83, SEQ ID NO: 85, SEQ ID NO: 7, or SEQ ID NO: 8. The polypeptide is modified by contacting the polypeptide with EDC and NHS.

As yet another example of a chemically crosslinked mutant C. difficile toxin, i.e., a polypeptide, the at least one amino acid may be chemically crosslinked by an agent that includes formaldehyde. Formaldehyde may react with the amino group of an N-terminal amino acid residue and the side-chains of arginine, cysteine, histidine, and lysine. Formaldehyde and glycine may form a Schiff-base adduct, which may attach to primary N-terminal amino groups, arginine, and tyrosine residues, and to a lesser degree asparagine, glutamine, histidine, and tryptophan residues.

A chemical crosslinking agent is said to reduce cytotoxicity of a toxin if the treated toxin has less toxicity (e.g., about 100%, 99%, 95%, 90%, 80%, 75%, 60%, 50%, 25%, or 10% less toxicity) than untreated toxin under identical conditions, as measured, for example, by an in vitro cytotoxicity assay, or by animal toxicity.

In one aspect, a immunogenic composition of the present invention comprises a polypeptide having the amino acid sequence SEQ ID NO: 4, wherein the methionine is not present (toxoid A), for example SEQ ID NO: 84, and a second polypeptide having the amino acid sequence SEQ ID NO: 6, wherein the methionine is not present (toxoid B), for example SEQ ID NO: 86, and an adjuvant, such as a CpG adjuvant or a saponin containing liposomal adjuvant.

In one aspect, immunogenic compositions comprising an effective amount of C. difficile toxoid A and toxoid B (e.g., from about 40 to about 500 μg/dose, such as about any of 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 μg/dose, such as about 50 to about 100 μg/dose (w/w, total amount of toxoids A and B in the composition)) at an effective toxoid A:B ratio (e.g., about any of 10%, 20%, 30%, 40%, 50%, 60%, 70%, 3:1, 3:2, or 1:1 toxoid A to toxoid B by weight), and with a sufficient purity (e.g., at least about 80 to about 100%, such as about any of 80, 85, 90, 95 or 90-100% (w/w)), using one or more administrations (e.g., at least two, three administrations or doses) by any suitable route (e.g., intramuscularly), each dose of a multiple dose administration regimen being suitably separated from one another (e.g., by at least about one to about ten days, such as about any of one, two, three, four, five, six, seven, eight, nine or ten, such as about seven days or by at least one to about seven months, such as about any one of one, two, three, four, five, six, or seven months) are provided. The length of time (time interval) between doses would be understood by those of ordinary skill to vary depending on the individual and that that interval should be long enough (e.g., as measured in days or months) such that the immune response from the prior dose both has time to develop (e.g., to be primed) and is not in any way inhibited by the subsequent dose (e.g., the boosting dose or doses).

In one aspect, the immunogenic composition used in the vaccination regimen of the present invention includes from about 40 to about 500 μg/dose of C. difficile toxoid A. In an aspect the composition includes from about 50 to about 400 μg/dose of C. difficile toxoid A. In one aspect, the composition includes from about 50 to about 200 μg/dose of C. difficile toxoid A. In one aspect the composition includes from about 50 to about 150 μg/dose. In one aspect the composition includes about any of 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 μg/dose of C. difficile toxoid A. In one aspect, the composition includes about 50 μg/dose of C. difficile toxoid A. In another aspect, the composition includes about 100 μg/dose of C. difficile toxoid A.

In one aspect the immunogenic composition used in the vaccination regimen of the present invention includes from about 40 to about 500 μg/dose of C. difficile toxoid B. In one aspect the composition includes from about 50 to about 400 μg/dose of C. difficile toxoid B. In one aspect the composition includes from about 50 to about 200 μg/dose of C. difficile toxoid B. In one aspect the composition includes from about 50 to about 150 μg/dose. In one aspect the composition includes about any of 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 μg/dose of C. difficile toxoid B. In one aspect, the composition includes about 50 μg/dose of C. difficile toxoid B. In another aspect, the composition includes about 100 μg/dose of C. difficile toxoid B.

In one aspect, the immunogenic composition used in the vaccination regimen of the present invention includes C. difficile toxoid A and B at the doses disclosed herein. In one aspect, the toxoid A to B ratio is 3:1, 3:2, or 1:1 toxoid A to toxoid B by weight. In one aspect, the toxoid A to B ratio is 1:3, 2:3, or 1:1 toxoid A to toxoid B by weight. In one aspect, the toxoid A to B ratio is 1:1 toxoid A to toxoid B by weight. In one aspect the composition used in the vaccination regimen of the present invention includes C. difficile toxoid A and B with a purity of at least about 80 to about 100%. In one aspect the composition used in the vaccination regimen of the present invention includes C. difficile toxoid A and B with a purity of at least about 90 to about 100%. In one aspect the composition used in the vaccination regimen of the present invention includes C. difficile toxoid A and B with a purity of about 80, 85, 90, 95 or 100% (w/w).

### Pharmaceutically Acceptable Carriers

In one aspect, the immunogenic compositions described herein may further comprise one or more pharmaceutically acceptable carriers and/or one or more adjuvants. In one aspect, the C. difficile toxoids A and/or B as described herein may be combined with one or more pharmaceutically acceptable carriers to provide a composition prior to administration. In one aspect, the composition, which may be a vaccine, may be provided as a lyophilized formulation that may be reconstituted at the clinical site with diluent, and/or mixed with adjuvant, when specified.

A pharmaceutically acceptable carrier is a material that is not biologically or otherwise undesirable, e.g., the material may be administered to a subject, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. Suitable pharmaceutical carriers and their formulations are described in, for example, Remington's: The Science and Practice of Pharmacy, 27^{th} Edition, David B. Troy, ed., Lippicott Williams & Wilkins (2005), and may be appropriate Typically, an appropriate amount of a pharmaceutically-acceptable salt is used in the formulation to render the formulation isotonic.

Examples of the pharmaceutically acceptable carriers include, but are not limited to, sterile water, saline, buffered solutions like Ringer's solution, and dextrose solution. The pH of the solution is generally from about 5 to about 8 or from about 7 to about 7.5. Other carriers include sustained-release preparations such as semipermeable matrices of solid hydrophobic polymers containing polypeptides or fragments thereof. Matrices may be in the form of shaped articles, e.g., films, liposomes or microparticles. It will be apparent to those persons skilled in the art that certain carriers may be more preferable depending upon, for instance, the route of administration and concentration of composition being administered. Carriers are those suitable for administration to humans or other subjects.

In one aspect, the composition includes a pharmaceutically acceptable carrier(s), which refer to any solvents, dispersion media, stabilizers, diluents, and/or buffers that are physiologically suitable. Exemplary stabilizers include carbohydrates, such as sorbitol, mannitol, starch, dextran, sucrose, trehalose, lactose, and/or glucose; inert proteins, such as albumin and/or casein; and/or other large, slowly metabolized macromolecules, such as polysaccharides such as chitosan, polylactic acids, polyglycolic acids and copolymers (such as latex functionalized SEPHAROSE™ agarose, agarose, cellulose, etc.), amino acids, polymeric amino acids, amino acid copolymers, and lipid aggregates (such as oil droplets or liposomes). Additionally, these carriers may function as immunostimulating agents (i.e., adjuvants).

Preferably, the C. difficile immunogenic composition includes trehalose. Preferred amounts of trehalose (% by weight) include from a minimum of about 1%, 2%, 3%, or 4% to a maximum of about 10%, 9%, 8%, 7%, 6%, or 5%. Any minimum value can be combined with any maximum value to define a suitable range. In one aspect, the composition includes about 3% to about 6% trehalose, most preferably, about 4.5% trehalose, for example, per 0.5 mL dose.

Exemplary buffers include phosphate (such as potassium phosphate, sodium phosphate); acetate (such as sodium acetate); succinate (such as sodium succinate); glycine; histidine; carbonate, Tris (tris(hydroxymethyl)aminomethane), and/or bicarbonate (such as ammonium bicarbonate) buffers.

Preferably, the C. difficile immunogenic composition includes a Tris buffer. Preferred amounts of Tris buffer include from a minimum of about 1 mM, 5 mM, 6 mM, 7 mM, 8 mM, 9 mM, 10 mM to a maximum of about 100 mM, 50 mM, 20 mM, 19 mM, 18 mM, 17 mM, 16 mM, 15 mM, 14 mM, 13 mM, 12 mM, or 11 mM. Any minimum value can be combined with any maximum value to define a suitable range. In one aspect, the composition includes about 10 mM to about 15 mM Tris buffer, more preferably, about 8 mM to about 12 mM Tris buffer, most preferably, about 10 mM Tris buffer, for example, per 0.5 mL dose.

In another aspect, the C. difficile immunogenic composition includes histidine buffer. Preferred amounts of histidine buffer include from a minimum of about 1 mM, 5 mM, 6 mM, 7 mM, 8 mM, 9 mM, 10 mM to a maximum of about 100 mM, 50 mM, 20 mM, 19 mM, 18 mM, 17 mM, 16 mM, 15 mM, 14 mM, 13 mM, 12 mM, or 11 mM. Any minimum value can be combined with any maximum value to define a suitable range. In one aspect, the composition includes about 10 mM to about 15 mM histidine buffer, more preferably, about 8 mM to about 12 mM histidine buffer most preferably, about 10 mM histidine buffer, for example, per 0.5 mL dose.

In another aspect, the C. difficile immunogenic composition includes phosphate buffer. Preferred amounts of phosphate buffer include from a minimum of about 1 mM, 5 mM, 6 mM, 7 mM, 8 mM, 9 mM, 10 mM to a maximum of about 100 mM, 50 mM, 2 mM, 19 mM, 18 mM, 17 mM, 16 mM, 15 mM, 14 mM, 13 mM, 12 mM, or 11 mM. Any minimum value can be combined with any maximum value to define a suitable range. In one aspect, the composition includes about 10 mM to about 15 mM phosphate buffer, more preferably, about 8 mM to about 12 mM phosphate buffer, most preferably, about 10 mM phosphate buffer, for example, per 0.5 mL dose. In some aspects, the composition includes a surfactant. Any surfactant is suitable, whether it is amphoteric, non-ionic, cationic or anionic. Exemplary surfactants include the polyoxyethylene sorbitan esters surfactants (e.g., TWEEN @), such as polysorbate 20 and/or polysorbate 80; polyoxyethylene fatty ethers derived from lauryl, cetyl, stearyl and oleyl alcohols (known as BRIJ surfactants), such as triethyleneglycol monolauryl ether (BRIJ 30); TRITON X 100, or t-octylphenoxypolyethoxyethanol; and sorbitan esters (commonly known as the SPANs), such as sorbitan trioleate (SPAN 85) and sorbitan monolaurate, and combinations thereof. Preferred surfactants include polysorbate 80 (polyoxyethylene sorbitan monooleate).

Polysorbate 80 (PS-80) is a non-ionic surfactant. In one aspect, the composition includes a PS-80 concentration ranging from 0.0005% to 1%. For example, the PS-80 concentration in the composition may be at least 0.0005%, 0.005%, 0.01%, 0.02%, 0.03%, 0.04%, 0.05%, 0.06%, 0.07%, 0.08%, 0.09%, 0.10%, 0.2%, 0.3%, 0.4%, 0.5%, 0.6%, 0.7%, 0.8%, 0.9%, 1%, or 1.1% PS-80. In one aspect, the PS-80 concentration in the composition may be at most 2.0%, 1.9%, 1.8%, 1.7%, 1.6%, 1.5%, 1.4%, 1.3%, 1.2%, 1.1%, 1.0%, 0.9%, 0.8%, or 0.7% PS-80. Any minimum value may be combined with any maximum value described herein to define a range. Preferably, the composition comprises about 0.01% PS-80.

In an exemplary aspect, the immunogenic composition of the present invention comprises C. difficile Toxoid A and Toxoid B in trehalose, Tris buffer and polysorbate 80. In another exemplary aspect, the immunogenic composition of the present invention is a lyophilized composition comprising C. difficile Toxoid A and Toxoid B in trehalose, Tris buffer and polysorbate 80 and is reconstituted with an adjuvant or adsorbent. For example, a CpG adjuvant comprising a CpG alone or a CpG and aluminum hydroxide, or a saponin containing liposomal adjuvant.

In another exemplary aspect, the immunogenic composition includes trehalose, tris buffer, histidine buffer and polysorbate 80. In another exemplary aspect, the immunogenic composition includes trehalose, tris buffer, sodium phosphate buffer, potassium phosphate buffer and polysorbate 80. The pH of the buffer will generally be chosen to stabilize the active material of choice, and can be ascertainable by those in the art by known methods. Preferably, the pH of the buffer will be in the range of physiological pH. Thus, preferred pH ranges are from about 3 to about 8; more preferably, from about 6.0 to about 8.0; yet more preferably, from about 6.5 to about 7.5.

The adjuvant may comprise, for instance, a suitable concentration (e.g., about any of 800-5000 μg/mL) of an adjuvant. The immunogenic compositions may further comprise aluminum (e.g., aluminum hydroxide or aluminum phosphate), for example, aluminum hydroxide in Sodium Chloride may be used as the diluent to reconstitute the lyophilized formulation. WFI may be used to dilute the lyophilized vaccine for the unadjuvanted formulations. The final dosing solution may comprise, for instance, composition/vaccine, diluent and adjuvant.

In one aspect, the pharmaceutical composition includes one, two or more different adjuvants. Alternatively, in one aspect, the composition is administered to the mammal in the absence of an adjuvant. That is, the composition does not comprise an adjuvant.

In some aspects, the pharmaceutical composition further includes formaldehyde. For example, in a preferred aspect, a pharmaceutical composition that further includes formaldehyde has an immunogenic composition, wherein the mutant C. difficile toxin of the immunogenic composition has been contacted with a chemical crosslinking agent that includes formaldehyde. The amount of formaldehyde present in the pharmaceutical composition may vary from a minimum of about 0.001%, 0.002%, 0.003%, 0.004%, 0.005%, 0.006%, 0.007%, 0.008%, 0.009%, 0.010%, 0.013%, or 0.015%, to a maximum of about 0.020%, 0.019%, 0.018%, 0.017% 0.016%, 0.015%, 0.014%, 0.013%, 0.012% 0.011% or 0.010%. Any minimum value can be combined with any maximum value to define a suitable range. In one aspect, the pharmaceutical composition includes about 0.010% formaldehyde.

In some alternative aspects, the pharmaceutical compositions described herein do not include formaldehyde. For example, in a preferred aspect, a pharmaceutical composition that does not include formaldehyde has an immunogenic composition, wherein at least one amino acid of the mutant C. difficile toxin is chemically crosslinked by an agent that includes EDC. More preferably, in such an aspect, the mutant C. difficile toxin has not been contacted with a chemical crosslinking agent that includes formaldehyde. As another exemplary aspect, a pharmaceutical composition that is in a lyophilized form does not include formaldehyde.

Also provided herein are kits for administering the C. difficile antigens. In one aspect, one or more of C. difficile antigens may form part of and/or be provided as a kit for administration to a subject. Instructions for administering the C. difficile antigens may also be provided by the kit. Compositions comprising C. difficile antigens as described herein may be included in a kit (e.g., a vaccine kit). For example, the kit may comprise a first container containing a composition described herein in dried or lyophilized form and a second container containing an aqueous solution for reconstituting the composition. The kit may optionally include the device for administration of the reconstituted liquid form of the composition (e.g., hypodermic syringe, microneedle array) and/or instructions for use. The device for administration may be supplied pre-filled with an aqueous solution for reconstituting the composition.

The volume of each delivered dose of study drug (vaccine or placebo) may be about 0.5 mL. The volume of each delivered dose of the composition disclosed herein may be about 0.2, 0.3, 0.4, 0.5, 0.6, 0.7; 0.8, 0.9 or 1 mL. The volume of each delivered dose of the composition disclosed herein may be about 0.4, 0.5, 0.6 ml. The volume of each delivered dose of the composition disclosed herein may be about 0.5 mL. The volume of each delivered dose of the composition disclosed herein may be about 1 mL. Formulations may be administered by any suitable route (e.g., subcutaneously, intravenously, intramuscularly, intraperitoneally, intradermally, intranodally, intranasally, orally).

As referred to above, an immunological composition is typically one that comprises C. difficile antigen(s) and, upon administration to a host/subject (e.g., a human), induces or enhances an immune response directed against the antigen (e.g., C. difficile). Such responses may include the generation of antibodies (e.g., through the stimulation of B cells) or a T cell-based response (e.g., a cytolytic response), which may be protective and/or neutralizing. A protective or neutralizing immune response may be one that is detrimental to the infectious organism corresponding to the antigen (e.g., from which the antigen was derived) and beneficial to the host (e.g., by reducing or preventing infection). As used herein, protective or neutralizing antibodies and/or cellular responses may be reactive with the C. difficile antigen(s) described here, especially when administered in an effective amount and/or schedule. Those antibodies and/or cellular responses may reduce or inhibit the severity, time, and/or lethality of C. difficile infection when tested in animals. As shown in the examples, the immunogenic compositions comprising adjuvants described herein may be used to induce an immune response against C. difficile. An immunological composition that, upon administration to a host, results in a therapeutic (e.g., typically administered during an active infection) and/or protective (e.g., typically administered before or after an active infection) and/or neutralizing immune response, may be considered a vaccine.

### Adjuvants

The present invention further provides for immunogenic compositions comprising an adjuvant. An adjuvant is a substance that enhances the immune response when administered together with an immunogen or antigen. Antigens may act primarily as a delivery system, primarily as an immune modulator or have strong features of both. Suitable adjuvants include those suitable for use in mammals, including humans. Preferred adjuvants augment the intrinsic immune response to an immunogen without causing conformational changes in the immunogen that may affect the qualitative form of the immune response. Suitable adjuvants include MPL™ (3-O-deacylated monophosphoryl lipid A; Corixa, Hamilton, MT), which is described in U.S. Pat. No. 4,912,094, which is hereby incorporated by reference in its entirety, an aluminum hydroxide gel such as ALHYDROGEL™ (Brenntag Biosector, Denmark); aluminum salts (such as aluminum hydroxide, aluminum phosphate, aluminum sulfate), which may be used with or without an immunostimulating agent such as MPL or 3-DMP, QS-21, polymeric or monomeric amino acids such as polyglutamic acid or polylysine.

Suitable adjuvants further include an immunostimulatory oligonucleotide such as a CpG oligonucleotide (see, e.g., WO 1998/040100, WO2010/067262 and further described herein), a saponin containing liposomal adjuvant (further described herein), or a saponin and an immunostimulatory oligonucleotide, such as a CpG oligonucleotide (see, e.g., WO 00/062800), which are incorporated by reference herein in their entireties.

Other adjuvants include RC-529, GM-CSF and Complete Freund's Adjuvant (CFA) and Incomplete Freund's Adjuvant (IFA). Yet another class of adjuvants is glycolipid analogues including N-glycosylamides, N-glycosylureas and N-glycosylcarbamates, each of which is substituted in the sugar residue by an amino acid.

An effective amount of an adjuvant, such as those described herein, refers to the amount necessary or sufficient to realize a desired biologic effect. For example, an effective amount of a adjuvant administered with an antigen for inducing an antigen-specific immune response is that amount necessary to induce an immune response in response to an antigen upon exposure to the antigen. Combined with the teachings provided herein, by choosing among the various adjuvants and weighing factors such as potency, relative bioavailability, subject body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the particular adjuvant being administered, the size of the subject, or the severity of the disease or condition.

### CpG Adjuvants

In some aspects, the immunogenic compositions described herein comprise C. difficile toxoids, TxdA and TxdB, and an immunostimulatory oligonucleotide adjuvant.

In a preferred aspect, the immunostimulatory oligonucleotide adjuvant is a CpG oligonucleotide, and most preferably a CpG oligodeoxynucleotides (CpG ODN), and accordingly these terms are used interchangeably unless otherwise indicated. A “CpG” refers to cytosine-phosphoguanosine (CpG) motif-containing oligodeoxynucleotide (CpG ODN), which is a toll-like receptor 9 (TLR9) agonist.

A CpG oligonucleotide is a short nucleic acid molecule containing a cytosine followed by a guanine linked by a phosphate bond in which the pyrimidine ring of the cytosine is unmethylated. A CpG motif is a pattern of bases that include an unmethylated central CpG surrounded by at least one base flanking (on the 3′ and the 5′ side of) the central CpG. CpG oligonucleotides include both D and K oligonucleotides. The entire CpG oligonucleotide may be unmethylated or portions may be unmethylated. Examples of CpG oligonucleotides useful in the methods provided by the present disclosure include those disclosed in U.S. Pat. Nos. 6,194,388, 6,207,646, 6,214,806, 628,371, 6,239,116, and 6,339,068.

CpG oligonucleotides may encompass various chemical modifications and substitutions, in comparison to natural RNA and DNA, involving a phosphodiester internucleoside bridge, a beta-D-ribose (deoxyhbose) unit and/or a natural nucleoside base (adenine, guanine, cytosine, thymine, uracil). Examples of chemical modifications are known to the skilled person and are described, for example in Uhlmann E. et al. (1990), Chem. Rev. 90:543; “Protocols for Oligonucleotides and Analogs”, Synthesis and Properties and Synthesis and Analytical Techniques, S. Agrawal, Ed., Humana Press, Totowa, USA 1993; Crooke, S T. et al. (1996) Annu. Rev. Pharmacol. Toxicol. 36:107-129; and Hunziker J. et al., (1995), Mod. Synth. Methods 7:331-417. Specifically, a CpG oligonucleotide can contain a modified cytosine. A modified cytosine is a naturally occurring or non-naturally occurring pyrimidine base analog of cytosine which can replace this base without impairing the immunostimulatory activity of the oligonucleotide. Modified cytosines include but are not limited to 5-substituted cytosines (e.g. 5-methyl-cytosine, 5-fluorocytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). Some of the preferred cytosines include 5-methyl-cytosine, 5-fluoro-cytosine, 5-hydroxy-cytosine, 5-hydroxymethyl-cytosine, and N4-ethyl-cytosine.

A CpG oligonucleotide can also contain a modified guanine. A modified guanine is a naturally occurring or non-naturally occurring purine base analog of guanine which can replace this base without impairing the immunostimulatory activity of the oligonucleotide. Modified guanines include but are not limited to 7-deeazaguanine, 7-deaza-7-substituted guanine, hypoxanthine, N2-substituted guanines (e.g. N2-methyl-guanine), 5-amino-3-methyl-3H,6H-thiazolo[4,5-d]pyhmidine-2,7-dione, 2,6-diaminopuhne, 2-aminopuhne, purine, indole, adenine, substituted adenines (e.g. N6-methyl-adenine, 8-oxo-adenine), 8-substituted guanine (e.g. 8-hydroxyguanine and 8-bromoguanine), and 6-thioguanine. In some aspects of the disclosure, the guanine base is substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom.

In certain aspects, the CpG oligonucleotides include modified backbones. It has been demonstrated that modification of the nucleic acid backbone provides enhanced activity of nucleic acids when administered in vivo. Secondary structures, such as stem loops, can stabilize nucleic acids against degradation. Alternatively, nucleic acid stabilization can be accomplished via phosphate backbone modifications. A preferred stabilized nucleic acid has at least a partial phosphorothioate modified backbone. Phosphorothioates may be synthesized using automated techniques employing either phosphoramidate or H-phosphonate chemistries. Aryl- and alkyl-phosphonates can be made, e.g. as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A. (1990) Chem. Rev. 90:544; Goodchild, J. (1990) Bioconjugate Chem. 1:165). 2′-O-methyl nucleic acids with CpG motifs also cause immune activation, as do ethoxy-modified CpG nucleic acids. In fact, no backbone modifications have been found that completely abolish the CpG effect, although it is greatly reduced by replacing the C with a 5-methyl C. Constructs having phosphorothioate linkages provide maximal activity and protect the nucleic acid from degradation by intracellular exo- and endo-nucleases.

In one aspect of the invention the oligonucleotide includes at least one phosphorothioate linkage. In another aspect all internucleotide linkages of the oligonucleotide are phosphorothioate linkages. In another aspect the oligonucleotide includes at least one phosphodiester-like linkage. In another aspect the phosphodiester-like linkage is a phosphodiester linkage. In another aspect a lipophilic group is conjugated to the oligonucleotide. In one aspect the lipophilic group is cholesterol.

In an embodiment, all the internucleotide linkage of the CpG oligonucleotides disclosed herein are phosphodiester bonds (“soft” oligonucleotides, as described in WO 2007/026190). In another embodiment, CpG oligonucleotides of the invention are rendered resistant to degradation (e.g., are stabilized). In an aspect, all the internucleotide linkage of the CpG oligonucleotides disclosed herein are phosphodiester bonds (“soft” oligonucleotides, as described in WO 2007/026190). In another aspect, CpG oligonucleotides of the invention are rendered resistant to degradation (e.g., are stabilized).

The immunostimulatory oligonucleotides may have a chimeric backbone, which have combinations of phosphodiester and phosphorothioate linkages. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. When the phosphodiester linkage is preferentially located within the CpG motif such molecules are called “semi-soft” as described in WO 2007/026190.

Other modified oligonucleotides include phosphodiester modified oligonucleotides, combinations of phosphodiester and phosphorothioate oligonucleotides, methylphosphonate, methyl phosphorothioate, phosphorordithioate, p-ethoxy, and combinations thereof. Each of these combinations and their particular effects on immune cells is discussed in more detail with respect to CpG nucleic acids in PCT Publication Nos. WO 96/02555 and WO 98/18810 and in U.S. Pat. Nos. 6,194,388 and 6,239,116.

Mixed backbone modified ODN may be synthesized as described in WO 2007/026190. In an aspect, the CpG oligonucleotides disclosed herein may comprise substitutions or modifications, such as in the bases and/or sugars as described in WO 2007/026190.

In some aspects of the invention, CpG-containing nucleic acids might be mixed with immunogenic carriers according to methods known to those skilled in the art (see, e.g., WO 03/024480).

The CpG oligonucleotides may have one or two accessible 5′ ends. It is possible to create modified oligonucleotides having two such 5 ends, for instance, by attaching two oligonucleotides through a 3′-3′ linkage to generate an oligonucleotide having one or two accessible 5′ ends. The 3′-3′-linkage may be a phosphodiester, phosphorothioate or any other modified internucleoside bridge. Methods for accomplishing such linkages are known in the art. For instance, such linkages have been described in Seliger, H. et al., Oligonucleotide analogs with terminal 3′-3′- and 5′-5′-internucleotidic linkages as antisense inhibitors of viral gene expression, Nucleosides and Nucleotides (1991), 10(1-3), 469-77 and Jiang, et al., Pseudo-cyclic oligonucleotides: in vitro and in vivo properties, Bioorganic and Medicinal Chemistry (1999), 7(12), 2727-2735.

Additionally, 3′-3′-linked oligonucleotides where the linkage between the 3′-terminal nucleosides is not a phosphodiester, phosphorothioate or other modified bridge, can be prepared using an additional spacer, such as tri- or tetra-ethyleneglycol phosphate moiety (Durand, M. et al., Triple-helix formation by an oligonucleotide containing one (dA)12 and two (dT)12 sequences bridged by two hexaethylene glycol chains, Biochemistry (1992), 31 (38), 9197-204, U.S. Pat. Nos. 5,658,738 and 5,668,265). Alternatively, the non-nucleotidic linker may be derived from ethanediol, propanediol, or from an abasic deoxyhbose (dSpacer) unit (Fontanel, Marie Laurence et al., Nucleic Acids Research (1994), 22(11), 2022-7) using standard phosphoramidite chemistry. The non-nucleotidic linkers can be incorporated once or multiple times, or combined with each other allowing for any desirable distance between the 3′-ends of the two oligonucleotides to be linked.

A phosphodiester internucleoside bridge located at the 3′ and/or the 5 end of a nucleoside can be replaced by a modified internucleoside bridge, wherein the modified internucleoside bridge is for example selected from phosphorothioate, phosphorodithioate, NR₁R₂-phosphoramidate, boranophosphate, a-hydroxybenzyl phosphonate, phosphate-(C₁-C₂₁)—O-alkyl ester, phosphate-[(C₆-C₂₁)aryl-(C₁-C₂₁)—O-alkyl]ester, (C₁-C₃)alkylphosphonate and/or (C₆-C₁₂)arylphosphonate bridges, (C₇-C₁₂)-a-hydroxymethyl-aryl (e.g. disclosed in PCT Publication No. WO 95/01363), wherein (C₆-C₁₂)aryl, (C₆-C₂₀)aryl and (C₆-C₁₄)aryl are optionally substituted by halogen, alkyl, alkoxy, nitro, cyano, and where R₁ and R₂ are, independently of each other, hydrogen, (C₁-C₁₈)-alkyl, (C₆-C₂₀)-aryl, (C₆-C₁₄)-aryl, (C₁-C₃)-alkyl, preferably hydrogen, (C₁-C₃)-alkyl, preferably (C₁-C₄)-alkyl and/or methoxyethyl, or R₁ and R₂ form, together with the nitrogen atom carrying them, a 5 to 6-membered heterocyclic ring which can additionally contain a further heteroatom selected from the group O, S and N.

The replacement of a phosphodiester bridge located at the 3′ and/or the 5′ end of a nucleoside by a dephospho bridge (dephospho bridges are described, for example, in Uhlmann E. and Peyman A. in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

Different classes of CpG immunostimulatory oligonucleotides have been identified and are described in greater detail in WO 2010/125480. Compositions and methods of the present invention include the use of these different classes of CpG immunostimulatory oligonucleotides. In aspects of the invention, the immunostimulatory oligonucleotides include, but are not limited to, oligonucleotides that are A-Class, B-Class, C-Class, T-Class, P-Class or any Class with an E modification.

In an aspect of the present invention, the immunogenic compositions as disclosed herein comprise an A class CpG ODN. In some aspects, the A class CpG oligonucleotide of the present invention comprises the nucleic acid sequence: 5′ GGGGACGACGTCGTGGGGGGG 3′ (SEQ ID NO: 847).

In any of the A class CpG oligonucleotide sequences, all of the linkages may be all phosphorothioate bonds. In another aspect, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

In a preferred aspect of the present invention, the immunogenic compositions as disclosed herein comprise a B class CpG ODN that preferentially activate B cells. In one aspect, the CpG oligonucleotide of the present invention is a B class CpG oligonucleotide represented by at least the formula: 5′ X₁X₂CGX₃X₄ 3′, wherein X1, X2, X3, and X4 are nucleotides. In one embodiment, X₂ is adenine, guanine, or thymine. In another embodiment, X₃ is cytosine, adenine, or thymine. The B class CpG oligonucleotide sequences of the present invention may include those described in WO 96/02555, WO 98/18810 and U.S. Pat. Nos. 6,194,388; 6,207,646; 6,214,806; 6,218,371; 6,239,116 and 6,339,068.

In some aspects, the B class CpG oligonucleotides of the present invention may include, but are not limited to, the following nucleic acid sequences:

(SEQ ID NO: 48; CpG 24555)
5′ TCGTCGTTTTTCGGTGCTTTT 3′,
(SEQ ID NO: 841; CpG 1018)
5′ TGACTGTGAACGTTCGAGATGA 3′;
(SEQ ID NO: 842; CpG 7909)
5′ TCGTCGTTTTGTCGTTTTGTCGTT 3′;
(SEQ ID NO: 843; CpG 10103)
5′ TCGTCGTTTTTCGGTCGTTTT 3′;
(SEQ ID NO: 844; CpG 1826)
5′ TCCATGACGTTCCTGACGTT 3′;
(SEQ ID NO: 845)
5′ TCGTCGTTTCGTCGTTTTGTCGTT 3′;
and
(SEQ ID NO: 846)
5′ TCGTCGTTTTGTCGTTTTTTTCGA 3′.

In any of the B class CpG oligonucleotide sequences, all of the linkages may be all phosphorothioate bonds. In another aspect, in any of these sequences, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

In a preferred aspect of the invention, the CpG ODN comprises the nucleic acid sequence 5′ T*C*G*T*C*G*T*T*T*T*T*C*G*G*T*G*C*T*T*T*T 3′ (SEQ ID NO: 48) wherein * indicates a phosphorothioate linkage. The CpG ODN of this sequence is known as CpG 24555, which is described in WO2010/067262. CpG 24555 is a TLR9 agonist with potent Th1 cell activity that stimulates strong B-cell and NK-cell activation.

In an aspect of the present invention, the immunogenic compositions as disclosed herein comprise a C class CpG oligonucleotide. In some aspects, the C class CpG oligonucleotides of the present invention may include, but are not limited to, the following nucleic acid sequences:

(SEQ ID NO: 848)
5′ TCGCGTCGTTCGGCGCGCGCCG 3′;
(SEQ ID NO: 849)
5′ TCGTCGACGTTCGGCGCGCGCCG 3′;
(SEQ ID NO: 850)
5′ TCGGACGTTCGGCGCGCGCCG 3′;
(SEQ ID NO: 851)
5′ TCGGACGTTCGGCGCGCCG 3′;
(SEQ ID NO: 852)
5′ TCGCGTCGTTCGGCGCGCCG 3′;
(SEQ ID NO: 853)
5′ TCGACGTTCGGCGCGCGCCG 3′;
(SEQ ID NO: 854)
5′ TCGACGTTCGGCGCGCCG 3′;
(SEQ ID NO: 855)
5′ TCGCGTCGTTCGGCGCCG 3′;
(SEQ ID NO: 856)
5′ TCGCGACGTTCGGCGCGCGCCG 3′;
(SEQ ID NO: 857)
5′ TCGTCGTTTTCGGCGCGCGCCG 3′;
(SEQ ID NO: 858)
5′ TCGTCGTTTTCGGCGGCCGCCG 3′;
(SEQ ID NO: 859)
5′ TCGTCGTTTTACGGCGCCGTGCCG 3′;
and
(SEQ ID NO: 860)
5′ TCGTCGTTTTCGGCGCGCGCCGT 3′

In any of the C class CpG oligonucleotide sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

In an aspect of the present invention, the immunogenic compositions as disclosed herein comprise a P class CpG Oligonucleotide. In some aspects, the CpG oligonucleotides of the present invention may include a P class CpG oligonucleotide containing a 5′ TLR activation domain and at least two palindromic regions, one palindromic region being a 5′ palindromic region of at least 6 nucleotides in length and connected to a 3′ palindromic region of at least 8 nucleotides in length either directly or through a spacer, wherein the oligonucleotide includes at least one YpR dinucleotide. In one aspect, the P class CpG oligonucleotide includes at least one unmethylated CpG dinucleotide. In another aspect, the TLR activation domain is TCG, TTCG, TTTCG, TYpR, TTYpR, TTTYpR, UCG, UUCG, UUUCG, TTT, orTTTT. In yet another aspect, the TLR activation domain is within the 5′ palindromic region. In another aspect, the TLR activation domain is immediately 5′ to the 5′ palindromic region. In some aspects, the P class CpG oligonucleotides of the invention comprise the nucleic acid sequence: 5′ TCGTCGACGATCGGCGCGCGCCG 3′ (SEQ ID NO: 861).

In any of the P class CpG oligonucleotide sequences, all of the linkages may be all phosphorothioate bonds. In another aspect, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

The immunogenic compositions comprising a CpG adjuvant may further comprise a buffer. Exemplary buffers include phosphate (such as potassium phosphate, sodium phosphate); acetate (such as sodium acetate); succinate (such as sodium succinate); glycine; histidine; carbonate, Tris (tris(hydroxymethyl)aminomethane), and/or bicarbonate (such as ammonium bicarbonate) buffers.

In a preferred aspect, the CpG adjuvant may include a histidine buffer. Preferred amounts of histidine buffer include a concentration from about 1 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 50 mM, or 100 mM. In one aspect, the CpG adjuvant includes histidine buffer at a concentration of about 5 mM to about 15 mM histidine buffer. In one aspect, the CpG adjuvant includes histidine buffer at a concentration of about 8 mM to about 12 mM. In a preferred aspect, the CpG adjuvant includes histidine buffer at a concentration of about 10 mM.

In another aspect, the CpG adjuvant may include a phosphate buffer. Preferred amounts of phosphate buffer include a concentration from about 1 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 50 mM, or 100 mM. In one aspect, the CpG adjuvant includes phosphate buffer at a concentration of about 10 mM to about 25 mM. In a preferred aspect, the CpG adjuvant includes phosphate buffer at a concentration of about 20 mM phosphate.

The CpG adjuvant may further comprise a salt. Exemplary salts include magnesium chloride, potassium chloride, sodium chloride and a combination thereof.

In a preferred aspect, the CpG adjuvant may include sodium chloride (NaCl). Preferred amounts of sodium chloride include a concentration from a minimum of about 10 mM, 20 mM, 25 mM, 30 mM, 40 mM, 50 mM, 60 mM, 75 mM, 80 mM, 90 mM, 100 mM, 125 mM, 150 mM, 175 mM, 200 mM, 225 mM, 250 mM, 270 mM, or 300 mM. In a preferred aspect, the CpG adjuvant includes sodium chloride at a concentration of about 20 to about 100 mM. In a preferred aspect, the CpG adjuvant includes sodium chloride at a concentration of about 40 to about 200 mM. In a preferred aspect, the CpG adjuvant includes sodium chloride at a concentration of about 50 mM. In another preferred aspect, the CpG adjuvant includes sodium chloride at a concentration of about 60 mM.

Doses of the CpG oligonucleotides describe herein may range from about 0.1 μg to 50 mg per dose depending on the application. In some aspects, the dose may range from about 10 μg to 10 mg per administration. In some aspects, the immunogenic composition comprises CpG alone. In some aspects, the immunogenic composition comprises CpG and an additional adjuvant.

In one aspect, the immunogenic composition comprises about 0.1 to 5 mg/mL or higher of CpG, including at least about 0.1 mg/mL, at least about 0.5 mg/mL, at least about 1.0 mg/mL, at least about 1.1 mg/mL, at least about 1.2 mg/mL, at least about 1.3 mg/mL, at least about 1.4 mg/mL, at least about 1.5 mg/mL, at least about 1.6 mg/mL, at least about 1.7 mg/mL, at least about 1.8 mg/mL, at least about 1.9 mg/mL, at least about 2.0 mg/mL, at least about 2.1 mg/mL, at least about 2.2 mg/mL, at least about 2.3 mg/mL, at least about 2.4 mg/mL, at least about 2.5 mg/mL, at least about 3.0 mg/mL, at least about 3.1 mg/mL, at least about 3.2 mg/mL, at least about 3.3 mg/mL, at least about 3.4 mg/mL, at least about 3.5 mg/mL, at least about 3.6 mg/mL, at least about 3.7 mg/mL, at least about 3.8 mg/mL, at least about 3.9 mg/mL, at least about 4.0 mg/mL, at least about 4.5 mg/mL, at least about 5.0 mg/mL or higher of CpG.

In one aspect, the immunogenic composition comprises about 0.1 to about 1.0 mg/mL of CpG. In one preferred aspect, the immunogenic composition comprises about 0.5 mg/mL of CpG. In one aspect, the immunogenic composition comprises about 0.1 to about 1.0 mg/mL of CpG 24555. In one preferred aspect, the immunogenic composition comprises about 0.5 mg/mL of CpG 24555.

In one aspect, the immunogenic composition comprises about 0.5 to about 1.5 mg/mL of CpG. In one preferred aspect, the immunogenic composition comprises about 1.0 mg/mL of CpG. In one aspect, the immunogenic composition comprises about 0.5 to about 1.5 mg/mL of CpG 24555. In one preferred aspect, the immunogenic composition comprises about 1.0 mg/mL of CpG 24555.

In one aspect, the immunogenic composition comprises about 0.8 to about 1.8 mg/mL of CpG. In one preferred aspect, the immunogenic composition comprises about 1.2 mg/mL of CpG. In one aspect, the immunogenic composition comprises about 0.8 to about 1.8 mg/mL of CpG 24555. In one preferred aspect, the immunogenic composition comprises about 1.2 mg/mL of CpG 24555.

In one aspect, the immunogenic composition comprises about 3.0 to about 4.0 mg/mL of CpG. In one preferred aspect, the immunogenic composition comprises about 3.6 mg/mL of CpG. In one aspect, the immunogenic composition comprises about 3.0 to about 4.0 mg/mL of CpG 24555. In one preferred aspect, the immunogenic composition comprises about 3.6 mg/mL of CpG 24555.

In a preferred aspect, an immunogenic composition comprises lyophilized C. difficile toxoid A and toxoid B, reconstituted with a CpG adjuvant comprising about 3.6 mg/mL of CpG 24555 (high-dose CpG). In a preferred aspect, an immunogenic composition comprises lyophilized C. difficile toxoids A and B, reconstituted with a CpG adjuvant comprising about 3.6 mg/mL of CpG 24555 in 10 mM histidine, 60 mM NaCl at pH 6.5 (high-dose CpG). In a preferred aspect, the composition is administered at a dose volume of 0.5 mL.

In a preferred aspect, an immunogenic composition comprises lyophilized C. difficile toxoid A and toxoid B at 0.4 mg/mL total (200 μg/mL of toxoid A and 200 μg/mL of toxoid B), reconstituted with a CpG adjuvant comprising about 3.6 mg/mL of CpG 24555 (high-dose CpG). In a preferred aspect, an immunogenic composition comprises lyophilized C. difficile toxoid A and toxoid B at 0.4 mg/mL total (200 μg/mL of toxoid A and 200 μg/mL of toxoid B), reconstituted with a CpG adjuvant comprising about 3.6 mg/mL of CpG 24555 in 10 mM histidine, 60 mM NaCl at pH 6.5 (high-dose CpG). In a preferred aspect, the composition is administered at a dose volume of 0.5 mL.

The immunogenic compositions comprising CpG adjuvants disclosed herein may further comprise an additional adjuvant.

In an aspect, the immunogenic compositions comprising CpG adjuvants disclosed herein may further comprise aluminum salts (alum) (e.g., aluminum phosphate, aluminum sulfate or aluminum hydroxide). In one aspect, the immunogenic compositions comprising CpG adjuvants further comprise aluminum phosphate or aluminum hydroxide as adjuvant. In a preferred aspect, the immunogenic compositions comprising CpG adjuvants further comprise aluminum hydroxide (Al(OH)₃).

In one aspect, the immunogenic compositions comprising CpG may further comprise about 0.1 to 5 mg/mL or higher of Al(OH)₃, including at least about 0.1 mg/mL, at least about 0.5 mg/mL, at least about 1.0 mg/mL, at least about 1.1 mg/mL, at least about 1.2 mg/mL, at least about 1.3 mg/mL, at least about 1.4 mg/mL, at least about 1.5 mg/mL, at least about 1.6 mg/mL, at least about 1.7 mg/mL, at least about 1.8 mg/mL, at least about 1.9 mg/mL, at least about 2.0 mg/mL, at least about 2.1 mg/mL, at least about 2.2 mg/mL, at least about 2.3 mg/mL, at least about 2.4 mg/mL, at least about 2.5 mg/mL, at least about 3.0 mg/mL, at least about 3.1 mg/mL, at least about 3.2 mg/mL, at least about 3.3 mg/mL, at least about 3.4 mg/mL, at least about 3.5 mg/mL, at least about 3.6 mg/mL, at least about 3.7 mg/mL, at least about 3.8 mg/mL, at least about 3.9 mg/mL, at least about 4.0 mg/mL, at least about 4.5 mg/mL, at least about 5.0 mg/mL or higher of Al(OH)₃.

In one aspect, the immunogenic compositions comprising CpG may further comprise about 0.5 to about 2.5 mg/mL of Al(OH)₃. In one aspect, the immunogenic compositions comprising CpG may further comprise about 1.0 to about 2.5 mg/mL of Al(OH)₃. In one aspect, the immunogenic compositions comprising CpG may further comprise about 1.5 to about 2.5 mg/mL of Al(OH)₃. In one preferred aspect, the immunogenic compositions comprising CpG may further comprise about 1.0 mg/mL of Al(OH)₃. In one preferred aspect, the immunogenic compositions comprising CpG may further comprise about 1.5 mg/mL of Al(OH)₃. In one preferred aspect, the immunogenic compositions comprising CpG may further comprise about 1.7 mg/mL of Al(OH)₃. In one preferred aspect, the immunogenic compositions comprising CpG may further comprise about 1.8 mg/mL of Al(OH)₃. In one preferred aspect, the immunogenic compositions comprising CpG adjuvants further comprise about 2.0 mg/mL of Al(OH)₃. In one preferred aspect, the immunogenic compositions comprising CpG adjuvants further comprise about 2.5 mg/mL of Al(OH)₃.

In one aspect, the immunogenic compositions comprise about 0.5 mg/mL or higher of CpG (e.g. CpG 24555) and 1.0 mg/mL or higher of Al(OH)₃.

In one aspect, the immunogenic compositions comprise about 0.5/1.0, 0.5/1.5, 0.5/1.6, 0.5/1.7, 0.5/1.8, 0.5/1.9, 0.5/2.0, 0.5/2.5 mg/mL or higher of CpG/Al(OH)₃. In one aspect, the immunogenic compositions comprise about 1.0/1.0, 1.0/1.5, 1.0/1.6, 1.0/1.7, 1.0/1.8, 1.0/1.9, 1.0/2.0, 1.0/2.5 mg/mL or higher of CpG/Al(OH)₃. In one aspect, the immunogenic compositions comprise about 1.1/1.0, 1.1/1.5, 1.1/1.6, 1.1/1.7, 1.1/1.8, 1.1/1.9, 1.1/2.0, 1.1/2.5 mg/mL or higher of CpG/Al(OH)₃. In one aspect, the immunogenic compositions comprise about 1.2/1.0, 1.2/1.5, 1.2/1.6, 1.2/1.7, 1.2/1.8, 1.2/1.9, 1.2/2.0, 1.2/2.5 mg/mL or higher of CpG/Al(OH)₃. In one aspect, the immunogenic compositions comprise about 1.3/1.0, 1.3/1.5, 1.3/1.6, 1.3/1.7, 1.3/1.8, 1.3/1.9, 1.3/2.0, 1.3/2.5 mg/mL or higher of CpG/Al(OH)₃. In one aspect, the immunogenic compositions comprise about 1.4/1.0, 1.4/1.5, 1.4/1.6, 1.4/1.7, 1.4/1.8, 1.4/1.9, 1.4/2.0, 1.4/2.5 mg/mL or higher of CpG/Al(OH)₃. In one aspect, the immunogenic compositions comprise about 1.5/1.0, 1.5/1.5, 1.5/1.6, 1.5/1.7, 1.5/1.8, 1.5/1.9, 1.5/2.0, 1.5/2.5 mg/mL or higher of CpG/Al(OH)₃. In one aspect, the immunogenic compositions comprise about 1.6/1.0, 1.6/1.5, 1.6/1.6, 1.6/1.7, 1.6/1.8, 1.6/1.9, 1.6/2.0, 1.6/2.5 mg/mL or higher of CpG/Al(OH)₃. In one aspect, the immunogenic compositions comprise about 1.7/1.0, 1.7/1.5, 1.7/1.6, 1.7/1.7, 1.7/1.8, 1.7/1.9, 1.7/2.0, 1.7/2.5 mg/mL or higher of CpG/Al(OH)₃. In one aspect, the immunogenic compositions comprise about 1.8/1.0, 1.8/1.5, 1.8/1.6, 1.8/1.7, 1.8/1.8, 1.8/1.9, 1.8/2.0, 1.8/2.5 mg/mL or higher of CpG/Al(OH)₃. In one aspect, the immunogenic compositions comprise about 1.9/1.0, 1.9/1.5, 1.9/1.6, 1.9/1.7, 1.9/1.8, 1.9/1.9, 1.9/2.0, 1.9/2.5 mg/mL or higher of CpG/Al(OH)₃. In one aspect, the immunogenic compositions comprise about 2.0/1.0, 2.0/1.5, 2.0/1.6, 2.0/1.7, 2.0/1.8, 2.0/1.9, 2.0/2.0, 2.0/2.5 mg/mL or higher of CpG/Al(OH)₃. In one aspect, the immunogenic compositions comprise about 2.1/1.0, 2.1/1.5, 2.1/1.6, 2.1/1.7, 2.1/1.8, 2.1/1.9, 2.1/2.0, 2.1/2.5 mg/mL or higher of CpG/Al(OH)₃. In one aspect, the immunogenic compositions comprise about 2.2/1.0, 2.2/1.5, 2.2/1.6, 2.2/1.7, 2.2/1.8, 2.2/1.9, 2.2/2.0, 2.2/2.5 mg/mL or higher of CpG/Al(OH)₃. In one aspect, the immunogenic compositions comprise about 2.3/1.0, 2.3/1.5, 2.3/1.6, 2.3/1.7, 2.3/1.8, 2.3/1.9, 2.3/2.0, 2.3/2.5 mg/mL or higher of CpG/Al(OH)₃. In one aspect, the immunogenic compositions comprise about 2.4/1.0, 2.4/1.5, 2.4/1.6, 2.4/1.7, 2.4/1.8, 2.4/1.9, 2.4/2.0, 2.4/2.5 mg/mL or higher of CpG/Al(OH)₃. In one aspect, the immunogenic compositions comprise about 2.5/1.0, 2.5/1.5, 2.5/1.6, 2.5/1.7, 2.5/1.8, 2.5/1.9, 2.5/2.0, 2.5/2.5 mg/mL or higher of CpG/Al(OH)₃.

In a preferred aspect, an immunogenic composition comprises lyophilized C. difficile toxoid A and toxoid B, reconstituted with a CpG adjuvant comprising about 1.0 mg/mL of CpG 24555 and about 1.5 mg/mL of Al(OH)₃ (low-dose CpG). In a preferred aspect, an immunogenic composition comprises lyophilized C. difficile toxoid A and toxoid B, reconstituted with a CpG adjuvant comprising about 1.0 mg/mL of CpG 24555 and about 1.5 mg/mL of Al(OH)₃ in 10 mM histidine, 50 mM NaCl at pH 6.5 (low-dose adjuvant). In a preferred aspect, the composition is administered at a dose volume of 0.5 mL.

In a preferred aspect, an immunogenic composition comprises lyophilized C. difficile toxoid A and toxoid B at 0.4 mg/mL total (200 μg/mL of toxoid A and 200 μg/mL of toxoid B), reconstituted with about 1.0 mg/mL of CpG 24555 and about 1.5 mg/mL of Al(OH)₃ (low-dose adjuvant). In a preferred aspect, an immunogenic composition comprises lyophilized C. difficile toxoid A and toxoid B total (200 μg/mL of toxoid A and 200 μg/mL of toxoid B), reconstituted with about 1.0 mg/mL of CpG 24555 and about 1.5 mg/mL of Al(OH)₃ in 10 mM histidine, 50 mM NaCl at pH 6.5 (low-dose adjuvant). In a preferred aspect, the composition is administered at a dose volume of 0.5 mL.

In one aspect, the immunogenic compositions comprise about 3.0/1.0, 3.0/1.5, 3.0/1.6, 3.0/1.7, 3.0/1.8, 3.0/1.9, 3.0/2.0, 3.0/2.5 mg/mL or higher of CpG/Al(OH)₃. In one aspect, the immunogenic compositions comprise about 3.1/1.0, 3.1/1.5, 3.1/1.6, 3.1/1.7, 3.1/1.8, 3.1/1.9, 3.1/2.0, 3.1/2.5 mg/mL or higher of CpG/Al(OH)₃. In one aspect, the immunogenic compositions comprise about 3.2/1.0, 3.2/1.5, 3.2/1.6, 3.2/1.7, 3.2/1.8, 3.2/1.9, 3.2/2.0, 3.2/2.5 mg/mL or higher of CpG/Al(OH)₃. In one aspect, the immunogenic compositions comprise about 3.3/1.0, 3.3/1.5, 3.3/1.6, 3.3/1.7, 3.3/1.8, 3.3/1.9, 3.3/2.0, 3.3/2.5 mg/mL or higher of CpG/Al(OH)₃. In one aspect, the immunogenic compositions comprise about 3.4/1.0, 3.4/1.5, 3.4/1.6, 3.4/1.7, 3.4/1.8, 3.4/1.9, 3.4/2.0, 3.4/2.5 mg/mL or higher of CpG/Al(OH)₃. In one aspect, the immunogenic compositions comprise about 3.5/1.0, 3.5/1.5, 3.5/1.6, 3.5/1.7, 3.5/1.8, 3.5/1.9, 3.5/2.0, 3.5/2.5 mg/mL or higher of CpG/Al(OH)₃. In one aspect, the immunogenic compositions comprise about 3.6/1.0, 3.6/1.5, 3.6/1.6, 3.6/1.7, 3.6/1.8, 3.6/1.9, 3.6/2.0, 3.6/2.5 mg/mL or higher of CpG/Al(OH)₃. In one aspect, the immunogenic compositions comprise about 3.7/1.0, 3.7/1.5, 3.7/1.6, 3.7/1.7, 3.7/1.8, 3.7/1.9, 3.7/2.0, 3.7/2.5 mg/mL or higher of CpG/Al(OH)₃. In one aspect, the immunogenic compositions comprise about 3.8/1.0, 3.8/1.5, 3.8/1.6, 3.8/1.7, 3.8/1.8, 3.8/1.9, 3.8/2.0, 3.8/2.5 mg/mL or higher of CpG/Al(OH)₃. In one aspect, the immunogenic compositions comprise about 3.9/1.0, 3.9/1.5, 3.9/1.6, 3.9/1.7, 3.9/1.8, 3.9/1.9, 3.9/2.0, 3.9/2.5 mg/mL or higher of CpG/Al(OH)₃. In one aspect, the immunogenic compositions comprise about 4.0/1.0, 4.0/1.5, 4.0/1.6, 4.0/1.7, 4.0/1.8, 4.0/1.9, 4.0/2.0, 4.0/2.5 mg/mL or higher of CpG/Al(OH)₃. In one aspect, the immunogenic compositions comprise about 4.5/1.0, 4.5/1.5, 4.5/1.6, 4.5/1.7, 4.5/1.8, 4.5/1.9, 4.5/2.0, 4.5/2.5 mg/mL or higher of CpG/Al(OH)₃. In one aspect, the immunogenic compositions comprise about 5.0/1.0, 5.0/1.5, 5.0/1.6, 5.0/1.7, 5.0/1.8, 5.0/1.9, 5.0/2.0, 5.0/2.5 mg/mL or higher of CpG/Al(OH)₃.

### Saponin Containing Liposomal Adjuvants

In some aspects, the immunogenic compositions described herein comprise C. difficile toxoids, TxdA and TxdB, and a saponin containing liposomal adjuvant.

The saponin containing liposomal adjuvant may include, but is not limited to, STIMULON™ (QS-21 (Quillaja Saponaria-21), which is a triterpene glycoside or saponin, Aquila, Framingham, Mass.) or particles generated therefrom such as ISCOMs (immune stimulating complexes) and ISCOMATRIX® adjuvant. Accordingly, the compositions of the present invention may be delivered in the form of ISCOMs, ISCOMS containing CTB, liposomes or encapsulated in compounds such as acrylates or poly(DL-lactide-co-glycoside) to form microspheres of a size suited to adsorption. Typically, the term “ISCOM” refers to immunogenic complexes formed between glycosides, such as triterpenoid saponins (particularly Quil A), and antigens which contain a hydrophobic region. In a preferred aspect, the adjuvant is an ISCOMATRIX adjuvant.

In a further preferred aspect, the saponin containing liposomal adjuvant comprises at least one saponin and a monophosphoryl lipid A (MPLA)-containing liposome composition. In one aspect, the saponin containing liposomal adjuvant comprises at least one saponin and a monophosphoryl lipid A (MPLA)-containing liposome composition, wherein the liposome composition comprises i) a lipid bilayer comprising phospholipids and ii) cholesterol.

In one aspect, the saponin may be selected from QS-7, QS-18, QS-21, or a mixture thereof. Preferably, the saponin is QS-21.

In one aspect, the phospholipid is selected from dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG). Preferably the phospholipids are DMPC and DMPG.

In a preferred aspect, the saponin containing liposomal adjuvant comprises QS-21, MPLA, DMPC, DMPG and cholesterol. In a preferred aspect, the saponin containing liposomal adjuvant comprises QS-21, MPLA, DMPC, DMPG and cholesterol in a phosphate buffer. MPLA is preferably Monophosphoryl 3-Deacyl Lipid A (also known as 3D-PHAD®). 3D-PHAD® is a synthetic analogue of MPLA derived from Salmonella minnesota. In a preferred aspect, the saponin containing liposomal adjuvant comprises QS-21, MPLA (3D-PHAD®), DMPC, DMPG and cholesterol.

In one aspect, the immunogenic composition comprises about 0.05 to about 1.0 mg/mL or higher of QS-21, including about 0.05 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL or about 1.0 mg/mL or higher of QS-21.

In one aspect, the immunogenic composition comprises about 0.1 to about 0.4 mg/mL of QS-21. In one preferred aspect, the immunogenic composition comprises about 0.2 mg/mL of QS-21.

In one aspect, the immunogenic composition comprises about 0.1 to about 1.0 mg/mL or higher of MPLA, including about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL or about 1.0 mg/mL or higher of MPLA (preferably Monophosphoryl 3-Deacyl Lipid A).

In one aspect, the immunogenic composition comprises about 0.2 to about 0.6 mg/mL of MPLA (preferably Monophosphoryl 3-Deacyl Lipid A).

In one preferred aspect, the immunogenic composition comprises about 0.3 to about 0.5 mg/mL of MPLA (preferably Monophosphoryl 3-Deacyl Lipid A).

In one preferred aspect, the immunogenic composition comprises about 0.4 mg/mL of MPLA (preferably Monophosphoryl 3-Deacyl Lipid A).

In one aspect, the immunogenic composition comprises about 0.5 to about 20 mg/mL or higher of cholesterol, including about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, about 10.0 mg/mL, about 11.0 mg/mL, about 12.0 mg/mL, about 13.0 mg/mL, about 14.0 mg/mL, about 15.0 mg/mL, about 16.0 mg/mL, about 17.0 mg/mL, about 18.0 mg/mL, about 19.0 mg/mL, about 20.0 mg/mL or higher of cholesterol.

In one aspect, the immunogenic composition comprises about 5 to about 15 mg/mL of cholesterol. In one preferred aspect, the composition comprises about 11 mg/mL of cholesterol.

In one aspect, the immunogenic composition comprises about 0.5 to about 20 mg/mL or higher of DMPC, including about 0.5 mg/mL, about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, about 10.0 mg/mL, about 11.0 mg/mL, about 12.0 mg/mL, about 13.0 mg/mL, about 14.0 mg/mL, about 15.0 mg/mL, about 16.0 mg/mL, about 17.0 mg/mL, about 18.0 mg/mL, about 19.0 mg/mL, about 20.0 mg/mL or higher of DMPC.

In one aspect, the immunogenic composition comprises about 5 to about 20 mg/mL of DMPC. In one aspect, the immunogenic composition comprises about 5 to about 15 mg/mL of DMPC. In one preferred aspect, the immunogenic composition comprises about 14 mg/mL of DMPC.

In one aspect, the immunogenic composition comprises about 0.5 to about 3.0 mg/mL or higher of DMPG, including about 0.5 mg/mL, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL or higher, at least about 2.5 mg/mL, or at least about 3.0 mg/mL or higher of DMPG.

In one aspect, the immunogenic composition comprises about 1.0 to about 2.0 mg/mL of DMPG. In one preferred aspect, the immunogenic composition comprises about 1.6 mg/mL of DMPG.

In a preferred aspect, the immunogenic composition comprising a saponin containing liposomal adjuvant comprises QS-21 at about 0.2 mg/mL, Monophosphoryl 3-Deacyl Lipid A (also known as 3D-PHAD®) at about 0.4 mg/mL, DMPC at about 14 mg/mL, DMPG about 1.6 mg/mL and cholesterol at about 11 mg/mL (LiNA-2).

In another aspect, the immunogenic composition comprising a saponin containing liposomal adjuvant comprises QS-21 at about 0.4 mg/mL, Monophosphoryl 3-Deacyl Lipid A (also known as 3D-PHAD®) at about 0.8 mg/mL, DMPC at about 28 mg/mL, DMPG about 3.2 mg/mL and cholesterol at about 22 mg/mL.

In another aspect, the immunogenic composition comprising a saponin containing liposomal adjuvant comprises QS-21 at about 0.1 mg/mL, Monophosphoryl 3-Deacyl Lipid A (also known as 3D-PHAD®) at about 0.2 mg/mL, DMPC at about 7 mg/mL, DMPG about 0.8 mg/mL and cholesterol at about 5.5 mg/mL.

In another aspect, the immunogenic composition comprising a saponin containing liposomal adjuvant may further comprise a buffer. Exemplary buffers include phosphate (such as potassium phosphate, sodium phosphate); acetate (such as sodium acetate); succinate (such as sodium succinate); glycine; histidine; carbonate, Tris (tris(hydroxymethyl)aminomethane), and/or bicarbonate (such as ammonium bicarbonate) buffers.

In a preferred aspect, the compositions comprising a saponin containing liposomal adjuvant may include a phosphate buffer. Preferred amounts of phosphate buffer include a concentration from 1 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 50 mM, or 100 mM. In one aspect, the saponin containing liposomal adjuvant includes phosphate buffer at a concentration of about 5 mM to about 15 mM. In one aspect, the saponin containing liposomal adjuvant includes phosphate buffer at a concentration of about 8 mM to 12 mM. In a preferred aspect, the saponin containing liposomal adjuvant includes phosphate buffer at a concentration of about 10 mM.

The saponin containing liposomal adjuvant may further comprise a salt. Exemplary salts include magnesium chloride, potassium chloride, sodium chloride and a combination thereof.

In a preferred aspect, the saponin containing liposomal adjuvant includes sodium chloride (NaCl). Preferred amounts of sodium chloride include a concentration from a minimum of about 10 mM, 20 mM, 25 mM, 30 mM, 40 mM, 50 mM, 60 mM, 75 mM, 80 mM, 90 mM, 100 mM, 125 mM, 150 mM, 175 mM, 200 mM, 225 mM, 250 mM, 270 mM, or 300 mM. In a preferred aspect, the saponin containing liposomal adjuvant includes sodium chloride at a concentration of about 100 to about 200 mM. In a preferred aspect, the saponin containing liposomal adjuvant includes sodium chloride at a concentration of about 150 mM.

In a preferred aspect, an immunogenic composition comprises lyophilized C. difficile toxoid A and toxoid B, reconstituted with a saponin containing liposomal adjuvant comprising about 0.2 mg/mL QS-21, about 0.4 mg/mL Monophosphoryl 3-Deacyl Lipid A (also known as 3D-PHAD®), about 14 mg/mL DMPC, about 1.6 mg/mL DMPG and about 11 mg/mL cholesterol (LiNA-2). In preferred aspect, an immunogenic composition comprises lyophilized C. difficile toxoid A and toxoid B, reconstituted with a saponin containing liposomal adjuvant comprising about 0.2 mg/mL QS-21, about 0.4 mg/mL Monophosphoryl 3-Deacyl Lipid A (also known as 3D-PHAD®), about 14 mg/mL DMPC, about 1.6 mg/mL DMPG and about 11 mg/mL cholesterol in 10 mM phosphate, 150 mM NaCl at pH 6.2 (LiNA-2). In a preferred aspect, the composition is administered at a dose volume of 0.5 mL.

In a preferred aspect, an immunogenic composition comprises lyophilized C. difficile toxoid A and toxoid B at 0.4 mg/mL total (200 μg/mL of toxoid A and 200 μg/mL of toxoid B), reconstituted with a saponin containing liposomal adjuvant comprising about 0.2 mg/mL QS-21, about 0.4 mg/mL Monophosphoryl 3-Deacyl Lipid A (also known as 3D-PHAD®), about 14 mg/mL DMPC, about 1.6 mg/mL DMPG and about 11 mg/mL cholesterol (LiNA-2). In preferred aspect, an immunogenic composition comprises lyophilized C. difficile toxoid A and toxoid B, reconstituted with a saponin containing liposomal adjuvant comprising about 0.2 mg/mL QS-21, about 0.4 mg/mL Monophosphoryl 3-Deacyl Lipid A (also known as 3D-PHAD®), about 14 mg/mL DMPC, about 1.6 mg/mL DMPG and about 11 mg/mL cholesterol in 10 mM phosphate, 150 mM NaCl at pH 6.2 (LiNA-2). In a preferred aspect, the composition is administered at a dose volume of 0.5 mL.

As use herein, “liposomes” refer to closed bilayer membranes containing an entrapped aqueous volume. Liposomes may also be uni-lamellar vesicles possessing a single membrane bilayer or multi-lamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Liposomes, as they are ordinarily used, consist of smectic mesophases, and can consist of either phospholipid or nonphospholipid smectic mesophases. Smectic mesophase is most accurately described by Small, HANDBOOK OF LIPID RESEARCH, Vol. 4, Plenum, N.Y., 1986, pp. 49-50.

The saponin containing liposomal adjuvant may be homogeneous or heterogeneous. As used herein, the term “homogeneous” shall mean a final adjuvant formulation comprising liposomes having a size range of 30-400 nm, as determined by methods known in the art including, but not limited to, Dynamic light scattering (DLS), Transmission electron cryomicroscopy (e.g. cryo-TEM or cryo-EM), Nanoparticle Tracking Analysis (NTA, e.g. ViewSizer). A “homogeneous” adjuvant formulation may also mean a final adjuvant formulation comprising liposomes having a polydispersity index (PDI) of between about 0.05 to 0.5 or between about 0.05 to about 0.3, preferably about 0.3.

As used herein, the term “heterogeneous” shall mean a final adjuvant formulation comprising liposomes having varying sizes ranging from 30 nm to over 10 micrometers, about 30 nm to 4 micrometers, about 30 nm to 1400 nm, preferably about 30 nm to 1000 nm as determined by methods known in the art including, but not limited to, Dynamic light scattering (DLS), Transmission electron microscopy (e.g. cryo-TEM or cryo-EM), Nanoparticle Tracking Analysis (NTA, e.g. ViewSizer). A “heterogeneous” adjuvant formulation may also mean a final adjuvant formulation comprising liposomes having a polydispersity index (PDI)>0.5. Calculations used for the determination of size and PDI parameters may be found in the ISO standard documents 13321:1996 E and ISO 22412:2008 (Worldwide M.I. Dynamic Light Scattering, Common Terms Defined. Malvern Instruments Limited; Malvern, UK: 2011. pp. 1-6. Inform White Paper). As used herein a “heterogeneous” adjuvant formulation shall also mean a “polydisperse” adjuvant formulation. The term “about” as used herein refers to ±5% of the referenced value.

In an aspect, the saponin containing liposomal adjuvant of the invention is homogeneous. In another aspect, the saponin containing liposomal adjuvant of the invention is heterogeneous.

Unless noted otherwise, the saponin containing liposomal adjuvant used in the Examples of the present application is a heterogeneous saponin containing liposomal adjuvant.

In one aspect, the saponin containing liposomal adjuvant is produced according to the process and methods described in U.S. Provisional Application Nos. 63/319,418 and 63/485,964, and International Publication No. WO2023/175454, which are incorporated by reference herein in their entireties. the disclosures of which are incorporated by reference herein in their entireties Multiple processes are described to prepare heterogeneous and homogeneous liposomal adjuvant formulations scalable for manufacturing.

In the present invention, the saponin containing liposomal adjuvant may be produced by a first method for producing a homogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG), and a combination thereof, and (ii) cholesterol, wherein the mole percent concentration of the cholesterol in the liposome composition is greater than 50% (mol/mol), or the molar ratio of cholesterol to phospholipid is greater than 1, said method comprising the steps of:
(i) dissolving the phospholipids, cholesterol and MPLA in an organic solvent to form an organic phase;
(ii) injecting the organic phase of step (i) into an aqueous phase at a specific flowrate and at a specific ratio of the organic phase to the aqueous phase to form a liposome;
(iii) stirring the liposome of step (ii) to form an intermediate liposome;
(iv) removing the organic phase of the intermediate liposome of step (iii);
(v) concentrating the intermediate liposome of step (iv); and
(vi) compounding the intermediate liposome of step (v) with a saponin to form a final adjuvant formulation having a size range of about 30-400 nm with a polydispersity of 0.05 to 0.5, thereby producing the homogeneous adjuvant formulation.

In one aspect of the first method for producing a homogeneous adjuvant composition, wherein in step (i) the phospholipids, cholesterol and MPLA are dissolved in the organic solvent by sonication, heat or a combination thereof, preferably by heating. In one aspect, the organic solvent is ethanol or isopropyl alcohol. In another aspect, the organic phase is heated to a temperature between 45° C. to 65° C.

In another aspect of the first method for producing a homogeneous adjuvant composition, the aqueous phase comprises water, and optionally, a buffer. In a preferred aspect, the buffer comprises 10 mM phosphate at pH 6.2 containing 150 mM NaCl. In a preferred aspect, the aqueous phase is 10 mM phosphate at pH 6.2 containing 150 mM NaCl. In another aspect, the aqueous phase is at a temperature between 20° C. to 60° C. In another aspect, the flowrate of step (ii) is 0.5 mL/min to 400 mL/min or quick addition. In another aspect, the flowrate of step (ii) is 0.5 mL/min to 400 mL/min. In another aspect, the injection of step (ii) is conducted by rapid injection of the organic phase of step (i).

In another aspect of the first method for producing a homogeneous adjuvant composition, the intermediate liposome of step (iii) is stirred at a rate of 700 rpm to 900 rpm.

In another aspect of the first method for producing a homogeneous adjuvant composition, the ratio of organic phase to aqueous phase of step (ii) ranges from 1:4 to 1:16. In a preferred aspect, the ratio is 1:8.

In another aspect of the first method for producing a homogeneous adjuvant composition, the size of the intermediate liposome in step (iv) is downsized by using a high pressure extruder or a microfluid homogenizer. In one aspect, the size of the liposome is downsized in step (iv) by using membrane sizes ranging from 50 nm to 120 nm or homogenization pressures between 17000 PSI and 24000 PSI, or a combination of both.

In another aspect of the first method for producing a homogeneous adjuvant composition, the organic solvent is removed before downsizing of step (iv) or after downsizing of step (iv). In one aspect, removing the organic phase of the intermediate liposome of step (iv) is by Tangential Flow Filtration (TFF). In another aspect, the TFF is TFF diafiltration. In another aspect, the TFF comprises membranes having a molecular weight cut-off (MWCO) ranging from 100-500 kDa.

In another aspect of the first method for producing a homogeneous adjuvant composition, the concentrating of step (v) is by Ultrafiltration. In one aspect, the Ultrafiltration comprises a bioburden reduction filter and a sterile filter.

In another aspect of the first method for producing a homogeneous adjuvant composition, the compounding of step (vi) is at a mixing speed of 300 rpm. In one aspect, the compounding of step (vi) ranges from 1 hour to 24 hours. In another aspect, the compounding of step (vi) occurs at room temperature or from 2-8° C.

In another aspect of the first method for producing a homogeneous adjuvant composition, the final adjuvant formulation has a size range of about 30 nm-400 nm.

In another aspect of the first method for producing a homogeneous adjuvant composition, the final adjuvant formulation has a polydispersity of 0.05 to 0.5.

In a further aspect of the first method for producing a homogeneous adjuvant composition, the injecting of step (ii) is by pump or syringe injection. In a preferred aspect, the injecting of step (ii) is by pump.

In one aspect, the saponin containing liposomal adjuvant may be produced by a second method for producing a homogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG) and a combination thereof, and (ii) cholesterol, wherein the mole percent concentration of the cholesterol in the liposome composition is greater than 50% (mol/mol), or the molar ratio of cholesterol to phospholipid is greater than 1, said method comprising the steps of:
(i) dissolving the phospholipids, cholesterol and MPLA in an organic solvent or a mixture of organic solvents to form an organic phase;
(ii) injecting the organic phase of step (i) into an aqueous phase at a specific flowrate and at a specific ratio of the organic phase to the aqueous phase to form an intermediate liposome;
(iii) concentrating the intermediate liposome of step (ii);
(iv) removing the organic phase of the intermediate liposome of step (iii);
(v) filtering the intermediate liposome of step (iv); and
(vi) compounding the intermediate liposome of step (v) with a saponin to form a final adjuvant formulation having a size range of about 30-400 nm with a polydispersity of 0.05 to 0.5, thereby producing the homogeneous adjuvant formulation.

In another aspect of the second method for producing a homogeneous adjuvant composition, in step (i) the phospholipids, cholesterol and MPLA are dissolved in the organic solvent by sonication, heat, stirring or a combination thereof. In one aspect, the organic solvent is ethanol or isopropyl alcohol or other organic solvents. In one aspect, the organic solvent is ethanol or isopropyl alcohol. In another aspect, the organic phase is heated to a temperature between 45° C. to 65° C. Preferably, between 50° C. to 65° C. or between 45° C. to 55° C.

In another aspect of the second method for producing a homogeneous adjuvant composition, the aqueous phase comprises water, and optionally, a buffer. In one aspect, the buffer comprises 10 mM phosphate at pH 6.2 containing 150 mM NaCl. In a preferred aspect, the aqueous phase is 10 mM phosphate at pH 6.2 containing 150 mM NaCl. In another aspect, the aqueous phase is at a temperature between 20° C. to 65° C. In another aspect, the flowrate of step (ii) is 5 mL/min to 15 mL/min. In another aspect, the flowrate of step (ii) is 12 mL/min. In another aspect, the intermediate liposome of step (ii) is stirred at a rate of 100 rpm to 1000 rpm. In another aspect, the ratio of organic phase to aqueous phase of step (ii) ranges from 1:2 to 1:16. In a preferred aspect, the ratio is 4:7.

In another aspect of the second method for producing a homogeneous adjuvant composition, the concentrating of step (iii) is by Ultrafiltration.

In another aspect of the second method for producing a homogeneous adjuvant composition, removing the organic phase of the intermediate liposome of step (iv) is by Tangential Flow Filtration (TFF). In one aspect, the TFF is TFF diafiltration. In another aspect, the TFF comprises membranes having a molecular weight cut-off (MWCO) ranging from 100-500 kDa.

In another aspect of the second method for producing a homogeneous adjuvant composition, the filtering of step (v) comprises a bioburden reduction filter and a sterile filter.

In another aspect of the second method for producing a homogeneous adjuvant composition, the compounding of step (vi) is at a mixing speed of 350 rpm for 1 hour at RT.

In another aspect of the second method for producing a homogeneous adjuvant composition, the final adjuvant formulation has a size range of about 50-200 nm. Preferably, the final adjuvant formulation has a size of about 100 nm.

In another aspect of the second method for producing a homogeneous adjuvant composition, the final adjuvant formulation has a polydispersity of 0.05 to 0.5. Preferably, the final adjuvant formulation has a PDI of <0.2.

In a further aspect of the second method for producing a homogeneous adjuvant composition, the injecting of step (ii) is by pump or syringe injection. In a preferred aspect, the injecting of step (ii) is by pump.

In another aspect, the saponin containing liposomal adjuvant may be produced by a first method for producing a heterogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the mole percent concentration of the cholesterol in the liposome composition is greater than 50% (mol/mol), or the molar ratio of cholesterol to phospholipid is greater than 1, said method comprising the steps of:
(i) dissolving the phospholipids, cholesterol and MPLA in an organic solvent to form a lipid solution as an organic phase;
(ii) simultaneously injecting the organic phase of step (i) and an aqueous phase together at specific flowrates and at a specific ratio of the organic solvent phase to the aqueous phase to form a liposome;
(iii) concentrating the intermediate liposome of step (ii);
(iv) removing the organic phase of the intermediate liposome of step (iii);
(v) treating the intermediate liposome of step (iv) by microfluidizer for 10 passes at −17500 psi;
(vi) filtering the intermediate liposome of step (v) using 0.22 um membrane; and
(vii) compounding the intermediate liposome of step (vi) with a saponin,
   thereby producing the heterogeneous adjuvant formulation.

In another aspect of the first method for producing a heterogeneous adjuvant formulation, in step (i) the phospholipids, cholesterol and MPLA are dissolved in the organic solvent by sonication, heat, stirring or a combination thereof. In one aspect, the organic solvent is ethanol or isopropyl alcohol or their mixture. In another aspect, the lipid formulation organic phase is heated to a temperature of 45-65° C.

In another aspect of the first method for producing a heterogeneous adjuvant formulation, the aqueous phase comprises water, and optionally, a buffer. In one aspect, the buffer comprises 10 mM phosphate at pH 6.2 containing 150 mM NaCl. In a preferred aspect, the aqueous phase is 10 mM phosphate at pH 6.2 containing 150 mM NaCl. In another aspect, the aqueous phase is at a temperature between 45-60° C.

In another aspect of the first method for producing a heterogeneous adjuvant formulation, in step (ii) the organic phase has a flowrate of 0.5 to 2 ml/min and the aqueous phase has a flowrate of 5 to 15 ml/min. In another aspect of the third method, in step (ii) the organic phase has a flowrate of 1.333 ml/min and the aqueous phase has a flowrate of 10.667 ml/min.

In another aspect of the first method for producing a heterogeneous adjuvant formulation, the intermediate liposome of step (iii) is stirred at a rate of 100 rpm to 900 rpm.

In another aspect of the first method for producing a heterogeneous adjuvant formulation, the ratio of organic phase to aqueous phase of step (ii) ranges from 1:4 to 1:8. In a preferred aspect, the ratio is 1:8.

In another aspect of the first method for producing a heterogeneous adjuvant formulation, removing the organic phase of the intermediate liposome of step (iii) and step (iv) is by Tangential Flow Filtration (TFF). In one aspect, the TFF is TFF ultrafiltration and diafiltration. In another aspect, the TFF comprises membranes having a molecular weight cut-off (MWCO) ranging from 100-500 kDa.

In another aspect of the first method for producing a heterogeneous adjuvant formulation, the concentrating of step (iii) is by Ultrafiltration.

In another aspect of the first method for producing a heterogeneous adjuvant formulation, the size of the intermediate liposome in step (iv) is treated by using a microfluidizer.

In another aspect of the first method for producing a heterogeneous adjuvant formulation, the organic solvent is removed before microfluidizing of step (v).

In another aspect of the first method for producing a heterogeneous adjuvant formulation, a buffer is added to the compounding of step (vii). In one aspect, the compounding of step (vii) is at a mixing speed of 350 rpm for 1 hour to 48 at RT or agitated for 1 hr. In another aspect, following the compounding of step (vii) the intermediate liposome is stored at RT without stirring for up to 48 hours.

In another aspect of the first method for producing a heterogeneous adjuvant formulation, the final adjuvant formulation has a size of about 300 nm to 1000 nm.

In another aspect of the first method for producing a heterogeneous adjuvant formulation, the final adjuvant formulation has a polydispersity of 0.4-1.0.

In a further aspect of the first method for producing a heterogeneous adjuvant formulation, the injecting of step (ii) is by Nanoassemblr or pump or syringe injection.

In another aspect, the saponin containing liposomal adjuvant may be produced by a second method for producing a heterogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the mole percent concentration of the cholesterol in the liposome composition is greater than 50% (mol/mol), or the molar ratio of cholesterol to phospholipid is greater than 1, said method comprising the steps of:
(i) dissolving the phospholipids, cholesterol and MPLA in an organic solvent to form a lipid formulation an organic phase;
(ii) injecting the organic phase of step (i) into an aqueous phase at a specific flowrate and at a specific ratio of the organic phase to the aqueous phase to form a liposome;
(iii) stirring the liposome of step (ii) to form an intermediate liposome;
(iv) concentrating the intermediate liposome of step (iii);
(v) removing the organic phase of the intermediate liposome of step (iv); and
(vi) compounding the intermediate liposome of step (vi) with a saponin,
   thereby producing the heterogeneous adjuvant formulation.

In another aspect of the second method for producing a heterogeneous adjuvant formulation, in step (i) the phospholipids, cholesterol and MPLA are dissolved in the organic solvent by sonication, heat, stirring or a combination thereof. In one aspect, the organic solvent comprises ethyl acetate and isopropyl alcohol. In another aspect, the lipid formulation organic phase is heated to a temperature from 50° C. to 65° C.

In another aspect of the second method for producing a heterogeneous adjuvant formulation, the aqueous phase comprises water, and optionally, a buffer. In one aspect, the buffer comprises 10 mM phosphate at pH 6.2 containing 150 mM NaCl. In a preferred aspect, the aqueous phase is 10 mM phosphate at pH 6.2 containing 150 mM NaCl. In another aspect, the aqueous phase is at a temperature of 20-25° C. In a further aspect, the flowrate of step (ii) is 10 to 30 mL/min. In a further aspect, the flowrate of step (ii) is 20 mL/min.

In another aspect of the second method for producing a heterogeneous adjuvant formulation, the intermediate liposome of step (iii) is stirred at a rate of 100 rpm to 900 rpm.

In another aspect of the second method for producing a heterogeneous adjuvant formulation, the ratio of organic phase to aqueous phase of step (ii) ranges from 1:4 to 1:8. In a preferred aspect, the ratio is 1.8.

In another aspect of the second method for producing a heterogeneous adjuvant formulation, the organic solvent is removed before compounding of step (vi).

In another aspect of the second method for producing a heterogeneous adjuvant formulation, removing the organic phase of the intermediate liposome of step (iv) and step (v) is by Tangential Flow Filtration (TFF). In one aspect, the TFF is TFF ultrafiltration and diafiltration.

In another aspect, the TFF comprises membranes having a molecular weight cut-off (MWCO) ranging from 100-500 kDa.

In another aspect of the second method for producing a heterogeneous adjuvant formulation, the concentrating of step (vi) is by Ultrafiltration.

In another aspect of the second method for producing a heterogeneous adjuvant formulation, a buffer is added to the compounding of step (vi).

In another aspect of the second method for producing a heterogeneous adjuvant formulation, the compounding of step (vi) is at a mixing speed of 300 rpm for 1 hour at RT. In one aspect, following the compounding of step (vii) the intermediate liposome is stored at RT without stirring for 24 hours.

In another aspect of the second method for producing a heterogeneous adjuvant formulation, the final adjuvant formulation has a size range of 300 nm to 1000 nm.

In another aspect of the second method for producing a heterogeneous adjuvant formulation, the final adjuvant formulation has a polydispersity of 0.4 to 1.0.

In another aspect of the second method for producing a heterogeneous adjuvant formulation, the injecting of step (ii) is by pipette, pump or syringe injection.

In another aspect, the saponin containing liposomal adjuvant may be produced by a third method for producing a heterogeneous adjuvant formulation comprising a liposome bilayer comprising (a) monophosphoryl lipid A (MPLA), (b) a saponin, and (c) a liposome composition comprising (i) at least one phospholipid selected from phosphatidylcholine (PC) and/or phosphatidylglycerol (PG), wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), distearyl phosphatidylglycerol (DSPG), and a combination thereof, and (ii) cholesterol, wherein the mole percent concentration of the cholesterol in the liposome composition is greater than 50% (mol/mol), or the molar ratio of cholesterol to phospholipid is greater than 1, said method comprising the steps of:
(i) preparing a lyophilized organic phase comprising the phospholipids, cholesterol and MPLA;
(ii) rehydrating the lyophilized organic phase of step (i) with an aqueous phase to form an intermediate liposome;
(iii) downsizing of the intermediate liposome of step (ii) using microfluidizer;
(iv) compounding the intermediate liposome of step (iii) with a saponin, thereby producing the heterogeneous adjuvant formulation.

In another aspect of the third method for producing a heterogeneous adjuvant formulation, in step (i) the phospholipids, cholesterol and MPLA are dissolved in the organic solvent by sonication, heat, stirring or a combination thereof. In one aspect, the organic solvent comprises tert-butyl alcohol (TBA) or its mixture. In another aspect, the lipid organic phase is heated to a temperature from 25° C. to 65° C. In another aspect, the organic phase solution is lyophilized.

In another aspect of the third method for producing a heterogeneous adjuvant formulation, the aqueous phase comprises water or a buffer. In one aspect, the buffer comprises 10 mM phosphate at pH 6.2 containing 150 mM NaCl. In a preferred aspect, the aqueous phase is 10 mM phosphate at pH 6.2 containing 150 mM NaCl. In another aspect, the aqueous phase is at a temperature from 20° C. to 70° C.

In another aspect of the third method for producing a heterogeneous adjuvant formulation, the intermediate liposome of step (ii) is stirred at a rate of 100-1000 rpm.

In another aspect of the third method for producing a heterogeneous adjuvant formulation, the size of the intermediate liposome in step (iii) is downsized with a microfluidizer and pressure at or about 18640 PSI.

In another aspect of the third method for producing a heterogeneous adjuvant formulation, the organic solvent is removed before rehydration of step (ii). In one aspect, removing the organic solvent is by lyophilization.

In another aspect of the third method for producing a heterogeneous adjuvant formulation, a buffer is added to the compounding of step (iv).

In another aspect of the third method for producing a heterogeneous adjuvant formulation, the compounding of step (iv) is at a mixing speed of 300 rpm or by agitation for 1 hour at RT.

In another aspect of the third method for producing a heterogeneous adjuvant formulation, the compounding of step (iv) the intermediate liposome is stored at RT without stirring for 24 hours.

In another aspect of the third method for producing a heterogeneous adjuvant formulation, the final adjuvant formulation has a size >300 nm.

In another aspect of the third method for producing a heterogeneous adjuvant formulation, the final adjuvant formulation has a polydispersity >0.4.

In one aspect of the methods for producing a homogeneous or heterogeneous adjuvant formulation, the saponin is selected from the group consisting of QS-7, QS-18, QS-21, or a mixture thereof. In a preferred aspect, the saponin is QS-21.

In one aspect of the methods for producing a homogeneous or heterogeneous adjuvant formulation, said at least one phospholipid is a mixture of dimyristoyl phosphatidylcholine (DMPC) and dimyristoyl phosphatidylglycerol (DMPG).

In one aspect, the liposome composition of the adjuvant formulation may comprise cholesterol at a mole percent concentration of over 50% (mol/mol), of about 55% to about 71% (mol/mol), or preferably about 55% (mol/mol).

In one aspect, the saponin containing liposomal adjuvant is a monophosphoryl lipid A (MPLA)-containing liposome composition comprising at least one saponin (e.g., QS-21) as described in U.S. Pat. No. 10,434,167 (e.g. ALFQ), which is hereby incorporated by reference in its entirety.

### Methods and Administration

The C. difficile toxoids disclosed herein may be used as antigens. For example, they may be part of a vaccine. Therefore, in one aspect, the immunogenic compositions of the invention are for use as a medicament. In an aspect, the immunogenic compositions of the invention are for use as a vaccine.

Therefore, in an aspect, the immunogenic compositions described herein, comprising C. difficile toxoid A and toxoid B, and an adjuvant, are for use in generating, inducing or eliciting an immune response in a subject. In one aspect, the subject is a mammal, such as a human, non-human primate, cat, sheep, pig, horse, bovine or dog. Preferably, the subject is a human.

The immunogenic compositions described herein, comprising C. difficile toxoid A and toxoid B, and an adjuvant, may be used in therapeutic or prophylactic methods for preventing, treating or ameliorating a bacterial infection, disease or condition in a human subject.

The immunogenic compositions described herein, comprising C. difficile toxoid A and toxoid B, and an adjuvant, may be used to prevent, treat or ameliorate a C. difficile infection, disease or condition in a human subject. The immunogenic compositions described herein comprising C. difficile toxoid A and toxoid B, and an adjuvant, may be used to prevent, treat or ameliorate an infection, disease or condition associated with C. difficile in a human subject. The immunogenic compositions described herein may be used to induce or elicit an immune response against C. difficile in a subject.

The immunogenic compositions described herein, comprising C. difficile toxoid A and toxoid B, and an adjuvant, may be used to prevent, treat or ameliorate a medically attended C. difficile infection, disease or condition in a human subject. The immunogenic compositions described herein may be used to prevent, treat or ameliorate medically attended C. difficile infection, disease or condition by the toxoids contained in the composition in a subject.

The immunogenic compositions described herein, comprising C. difficile toxoid A and toxoid B, and an adjuvant, may be used to generate, induce or elicit an immune response against C. difficile in a subject to prevent, treat or ameliorate a medically attended C. difficile infection, comprising administering to the subject an immunogenic composition of the present disclosure.

In one aspect, the invention relates to a method of preventing, treating or ameliorating an infection, disease or condition associated with C. difficile in a human subject, comprising administering to the subject an immunogenic composition of the present disclosure.

In another aspect, the invention relates to a method of preventing, treating or ameliorating a medically attended C. difficile infection in a human subject, comprising administering to the subject an immunogenic composition of the present disclosure.

In one aspect, the invention relates to a method of inducing an immune response against C. difficile in a human. In another aspect, the invention relates to a method of vaccinating a human. In one aspect, the method includes administering to the human at least one dose of an immunogenic composition described herein. In one aspect, the method includes administering to the human one dose of an immunogenic composition described herein. In one aspect, the method includes administering to the human two doses of an immunogenic composition described herein.

In another aspect, the method includes administering to the human a first dose and a second dose of an immunogenic composition described herein.

In one aspect, the second dose is administered about 6 months after the first dose, such as, for example, in a Month 0, 6 immunization schedule. In one aspect, the second dose is administered at least 20, 30, 50, 60, 100, 120, 160, 170, or 180 days after the first dose, and at most 250, 210, 200, or 190 days after the first dose.

In one aspect, the second dose is administered about 2 months after the first dose, such as, for example, in a Month 0, 2 immunization schedule. In one aspect, the second dose is administered at least 5, 10, 20, 30, 50, or 60 days after the first dose, and at most 150, 90, 80, or 70 days after the first dose. Any minimum value may be combined with any maximum value described herein to define a range.

In another aspect, the second dose is administered about 30 days (about 1 month) after the first dose. In another aspect, the second dose is administered about 60 days (about 2 months) after the first dose, such as, for example, in a Month 0, 2 immunization schedule. In another aspect, the second dose is administered about 180 days (about 6 months) after the first dose, such as, for example, in a Month 0, 6 immunization schedule. In yet another aspect, the second dose is administered about 120 days (about 4 months) after the first dose, such as, for example, in a Month 0, 4 immunization schedule.

In one aspect, the method includes administering to the human a single dose of the immunogenic composition and at most two doses. In one aspect, the method includes administering to the human two doses of the immunogenic composition and at most two doses.

In one aspect, the two doses are administered within a period of about 6 months after the first dose. In one aspect, the two doses are administered within a period of about 2 months after the first dose. Administering to the human at most two doses of the immunogenic composition may be advantageous. Such advantages include, for example, facilitating a human to comply with a complete administration schedule and facilitating cost-effectiveness of the schedule.

In one aspect, the method includes further administration of a booster dose to the human after the second dose. For example, a booster does may be administered 6 months or 12 months after the second dose. Further boosters may be administered. In one aspect, the method does not include further administration of a booster to the human after the second dose. A “booster” as used herein refers to an additional administration of the immunogenic composition to the human.

In one aspect, the method includes administering to the human three doses of an immunogenic composition described herein. In one aspect the method includes administering at most three doses of the immunogenic composition. In one aspect, the three doses are administered within a period of about 6 months after the first dose. In a further aspect, at most three doses within a period of about 6 months are administered to the human.

In one aspect, the second dose is administered about 30 days (about 1 month) after the first dose, and the third dose is administered about 150 to 180 days after the second dose, such as, for example, in a Month 0, 1, 6 immunization schedule. In another aspect, the second dose is administered about 60 days (about 2 months) after the first dose, and the third dose is administered about 120 to 150 days after the second dose, such as, for example, in a Month 0, 2, 6 month immunization schedule.

In one aspect, the first dose, second dose, and third dose are administered to the human over a period of about 150, 160, 170, or 180 days, and at most 240, 210 200, or 190 days. Any minimum value may be combined with any maximum value described herein to define a range. In another aspect, the first dose, second dose, and third dose is administered to the human over a period of about 180 days or 6 months. For example, the second dose may be administered to the human about 30 days after the first dose, and the third dose may be administered to the human about 120 days after the second dose. Accordingly, a schedule of administration includes administering a dose to the human at about months 0, 1, and 6. For example, the second dose may be administered to the human about 60 days after the first dose, and the third dose may be administered to the human about 120 days after the second dose. Accordingly, a schedule of administration includes administering a dose to the human at about months 0, 2, and 6.

In one aspect, the method includes an administration of a booster dose to the human after the third dose. For example, a booster does may be administered 6 months or 12 months after the third dose. Further boosters may be administered. In another aspect, the method does not include administration of a booster dose to the human after the third dose.

As described above, multiple doses of the immunogenic composition may be administered to the human, and the number of days between each dose may vary. An advantage of the method includes, for example, flexibility for a human to comply with the administration schedules.

Upon administration of compositions described herein using such methods to a host/subject, an immune response is typically observed, which typically includes a humoral immune response and may involve a cellular immune response.

In certain aspects, the method may comprise administering the immunogenic composition to a human, subject at risk for infection. In some aspects, the human subject may be at least about any of 40, 50, 55, 65, 75, 80 or 85 years or older. In some aspects, the human subject may be about 40 to about 65 years of age or older. In some aspects, the human subject may be about 65 to about 85 years of age older. In some aspects, the human subject may be about 18 years of age or older. In some aspects, the human subject may be about 50 years of age or older. In some aspects, the human subject may be about 55 years of age or older. In some aspects, the human subject may be about 60 years of age or older. In some aspects, the human subject may be about 65 years of age or older.

In one aspect, the invention relates to methods for immunizing a subject (e.g., a human being) against C. difficile by administering thereto a composition comprising one or more antigens of C. difficile and an adjuvant. In one aspect, the invention relates to a composition disclosed herein for use in a method for immunizing a subject against C. difficile. In one aspect, the invention relates to a composition disclosed herein for use in a method for immunizing a human subject against C. difficile. In one aspect, the human subject is 40-90 years of age or older. In one aspect, the human subject is 50-85 years of age or older. In one aspect, the human subject is 60-85 years of age or older. In an aspect, the human subject is 65-85 years of age or older. In one aspect, the human subject is 65-69 years of age or older. In an aspect, the human subject is 70-79 years of age or older. In one aspect, the human subject is 75-79 years of age or older. In one aspect, the human subject is at least 50, 55, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 years of age or older. In one aspect, the human subject is at least 65, 66, 67, 68, 69, 70, 71, 72, 73, 74, 75, 76, 77, 78, 79, 80, 81, 82, 83, 84 or 85 years of age or older.

The immunogenic compositions described above may include one mutant C. difficile toxin (A or B), i.e., polypeptides and an adjuvant. Accordingly, the immunogenic compositions can occupy separate vials (e.g., a separate vial for a composition including mutant C. difficile toxin A and a separate vial for a composition including mutant C. difficile toxin B) in the preparation or kit. The immunogenic compositions may be intended for simultaneous, sequential, or separate use.

In another aspect, the immunogenic compositions described above may include both mutant C. difficile toxins (A and B), i.e., polypeptides. Any combination of mutant C. difficile toxin A and mutant C. difficile toxin B described may be combined for an immunogenic composition. Accordingly, the immunogenic compositions can be combined in a single vial (e.g., a single vial containing both a composition including mutant C. difficile TcdA and a composition including mutant C. difficile TcdB). Preferably, the immunogenic compositions include a mutant C. difficile TcdA and a mutant C. difficile TcdB, i.e., polypeptides.

In certain aspects, it is preferred that the compositions described herein exhibit immunogenic properties (e.g., inducing a detectable and/or neutralizing and/or protective immune response) following appropriate administration to a subject. The presence of neutralizing and/or protective immune response may be demonstrated as described above and/or by showing that infection by a pathogen (e.g., C. difficile) is affected (e.g., decreased) in individuals (e.g., human being or other animal) to whom the materials described herein have been administered as compared to individuals to whom the materials have not been administered. For instance, one or more test subjects (e.g., human or non-human) may be administered by any suitable route and schedule a composition described herein, and then after a suitable amount of time (e.g., about 1, 2, 3, 4, 5, 6, 7, 8, 9, or 10 weeks) challenged by a pathogenic organism. The animal(s) may be monitored for immune function (e.g., antibody production, T cell activity) following administration and/or challenge. Sera may be analyzed for total antibody response or for expression of particular subtypes using, for example, an antibody ELISA and/or a pathogen neutralization assay. T cell activity may be measured by, for example, measuring IFN-γ production after re-stimulation with the antigen. Statistical analysis (e.g., Fisher's exact test, Wilcoxon test, Mann-Whitney Test) may then be performed on data to determine whether the effectiveness of the material in affecting the immune response.

### Toxin Neutralizing Activity

Immune response induced by administering the immunogenic compositions of the present invention may be determined using a toxin neutralization assay (TNA), ELISA, or more preferably, a cytotoxicity assay, such as that described in WIPO Patent Application WO/2012/143902, U.S. Pat. No. 9,187,536, and WIPO Patent Application WO/2014/060898, which are each incorporated by reference herein in their respective entireties.

In one aspect, the immune response induced in the human is neutralizing against a C. difficile strain that expresses a toxin A having an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the toxoid A of the composition.

In another aspect, the immune response induced in the human is neutralizing against a C. difficile strain that expresses a toxin B including an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the toxoid B of the composition.

The usefulness (e.g., immunogenicity) of any of the materials (e.g., compositions) and/or methods described herein may be assayed by any of the variety of methods known to those of skill in the art. Any one or more of the assays described herein, or any other one or more suitable assays, may be used to determine the suitability of any of the materials described herein for an intended purpose. It is to be understood that these methods are exemplary and non-limiting; other assays may also be suitable. For instance, the compositions described herein typically induce and/or enhance the production of antibodies against C. difficile upon administration to a subject. Such antibodies may be detected in the subject using any of the methods available to those of ordinary skill in the art. For instance, as described in the Examples section, serum may be obtained from a subject and tested by ELISA to detect immunoglobulin type G (IgG) antibodies to C. difficile toxin A and/or toxin B (e.g., “primary immunogenicity data”). Antibodies present in test sera may be reacted with toxin A or B antigens adsorbed to individual wells of a microtiter plate. The amount of antibody bound to the antigen coated wells may be determined using a colorimetric substrate reaction after binding of a secondary anti-IgG (e.g., anti-human IgG) antibody-enzyme conjugate. Substrate for the enzyme is then typically added that causes colorimetric change that was directly proportional to the antibody bound to the antigen. The concentration of antibodies in serum may be derived by extrapolation from a standard curve, which was generated from multiple dilutions of a reference standard serum with defined IgG units (ELISA unit (EU)/mL)). A toxin neutralization assay (TNA) may also be used to quantitate neutralizing antibodies to C. difficile toxin. In this assay, serial diluted serum may be incubated with a fixed amount of C. difficile toxin A or B. Test cells (e.g., Vero cells) may then then added and serum-toxin-cell mixture incubated under appropriate conditions (e.g., 37° C. for 6 days). The ability of the sera to neutralize the cytotoxic effect of the C. difficile toxin may be determined by and correlated to the viability of the cells. The assay utilizes the accumulation of acid metabolites in closed culture wells as an indication of normal cell respiration. In cells exposed to toxin, metabolism and CO₂ production is reduced; consequently, the pH rises (e.g., to 7.4 or higher) as indicated by the phenol red pH indicator in the cell culture medium. At this pH, the medium appears red. Cell controls, or cells exposed to toxin which have been neutralized by antibody, however, metabolize and produce CO₂ in normal amounts; as a result, the pH is maintained (e.g., at 7.0 or below) and at this pH, the medium appears yellow. Therefore, C. difficile toxin neutralizing antibodies correlate with the ability of the serum to neutralize the metabolic effects of C. difficile toxin on cells as evidenced by their ability to maintain a certain pH (e.g., of 7.0 or lower). The color change of the media may be measured (e.g., at 562 nm to 630 nm) using a plate reader to further calculate the antitoxin neutralizing antibody titer at 50% inhibition of the C. difficile toxin-mediated cytotoxicity. In one aspect, the composition induces a toxin neutralizing antibody titer that is at least greater than 1-fold, such as, for example, at least 1.01-fold, 1.1-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 32-fold, or higher in the human after receiving a dose of the composition than a toxin neutralizing antibody titer in the human prior to receiving said dose, when measured under identical conditions in a toxin neutralization assay.

The toxin neutralization assay (TNA) utilized for the Examples provided herein is described. Briefly, a 384-well microtiter plate was seeded with IMR-90 cells serving as the target of toxin-mediated cytotoxicity. Each test serum sample was analyzed separately for the ability to neutralize Toxin A or Toxin B. Four serial dilutions of test sera were mixed with fixed concentrations of Toxin A (TcdA) or Toxin B (TcdB) for 60 minutes in a humidified incubator (37° C./5% CO₂) to allow for neutralization of the toxins to occur. All plates included a reference standard and quality controls which consisted of antitoxin antibodies of known titer to monitor assay performance. After the 60-minute incubation, the toxin-antiserum mixture was applied to the IMR-90 cell monolayers and the plates were incubated for an additional 72 hours. Viability of the IMR-90 cell monolayers was then tested using the luciferase-based CellTiter-Glo® reagent which provides a measure of ATP levels in metabolically active cells and was reported as relative luminescence units (RLU). A high ATP level indicates high cell viability and antibody mediated neutralization of TcdA or TcdB. The neutralizing antibody concentration was determined by comparing the RLU value of a test sample to the calibration curve from the antitoxin A or B reference standard using a custom Statistical Analysis System (SAS®) program. The functional antibody concentrations were expressed as arbitrary units per mL (or neutralizing units/mL) of serum. The lower limit of quantitation (LLOQ) for the TcdA and TcdB TNA assays are 75.9 and 249.7 neutralizing units/mL of serum, respectively.

### Sequence Identifiers

SEQ ID NO: 1 sets forth the amino acid sequence for wild-type C. difficile 630 toxin A (TcdA).
SEQ ID NO: 2 sets forth the amino acid sequence for wild-type C. difficile 630 toxin B (TcdB).
SEQ ID NO: 3 sets forth the amino acid sequence for a mutant TcdA having a mutation at positions 285 and 287, as compared to SEQ ID NO: 1.
SEQ ID NO: 4 sets forth the amino acid sequence for a mutant TcdA having a mutation at positions 285, 287, and 700, as compared to SEQ ID NO: 1.

(SEQ ID NO: 4)
MSLISKEELIKLAYSIRPRENEYKTILTNLDEYNKLTINNNENKYLQLKKLNESIDVEMNKYKTSSRNRA
LSNLKKDILKEVILIKNSNTSPVEKNLHFVWIGGEVSDIALEYIKQWADINAEYNIKLWYDSEAFLVNTL
KKAIVESSTTEALQLLEEEIQNPQFDNMKFYKKRMEFIYDRQKRFINYYKSQINKPTVPTIDDIIKSHLV
SEYNRDETVLESYRTNSLRKINSNHGIDIRANSLFTEQELLNIYSQELLNRGNLAAASDIVRLLALKNEG
GVYLAVAMLPGIHSDLFKTISRPSSIGLDRWEMIKLEAIMKYKKYINNYTSENFDKLDQQLKDNFKLIIE
SKSEKSEIFSKLENLNVSDLEIKIAFALGSVINQALISKQGSYLTNLVIEQVKNRYQFLNQHLNPAIESD
NNFTDTTKIFHDSLENSATAENSMELTKIAPYLQVGEMPEARSTISLSGPGAYASAYYDFINLQENTIEK
TLKASDLIEFKEPENNLSQLTEQEINSLWSFDQASAKYQFEKYVRDYTGGSLSEDNGVDENKNTALDKNY
LLNNKIPSNNVEEAGSKNYVHYIIQLQGDDISYEATCNLESKNPKNSIIIQRNMNESAKSYFLSDDGESI
LELNKYRIPERLKNKEKVKVTFIGHGKDEENTSEFARLSVDSLSNEISSELDTIKLDISPKNVEVNLLGA
NMFSYDENVEETYPGKLLLSIMDKITSTLPDVNKNSITIGANQYEVRINSEGRKELLAHSGKWINKEEAI
MSDLSSKEYIFFDSIDNKLKAKSKNIPGLASISEDIKTLLLDASVSPDTKFILNNLKLNIESSIGDYIYY
EKLEPVKNIIHNSIDDLIDEFNLLENVSDELYELKKLNNLDEKYLISFEDISKNNSTYSVRFINKSNGES
VYVETEKEIFSKYSEHITKEISTIKNSIITDVNGNLLDNIQLDHTSQVNTLNAAFFIQSLIDYSSNKDVL
NDLSTSVKVQLYAQLFSTGLNTIYDSIQLVNLISNAVNDTINVLPTITEGIPIVSTILDGINLGAAIKEL
LDEHDPLLKKELEAKVGVLAINMSLSIAATVASIVGIGAEVTIFLLPIAGISAGIPSLVNNELILHDKAT
SVVNYFNHLSESKKYGPLKTEDDKILVPIDDLVISEIDENNNSIKLGTCNILAMEGGSGHTVTGNIDHFF
SSPSISSHIPSLSIYSAIGIETENLDESKKIMMLPNAPSRVFWWETGAVPGLRSLENDGTRLLDSIRDLY
PGKFYWRFYAFFDYAITTLKPVYEDTNIKIKLDKDTRNFIMPTITTNEIRNKLSYSFDGAGGTYSLLLSS
YPISTNINLSKDDLWIENIDNEVREISIENGTIKKGKLIKDVLSKIDINKNKLIIGNQTIDESGDIDNKD
RYIFLTCELDDKISLIIEINLVAKSYSLLLSGDKNYLISNLSNIIEKINTLGLDSKNIAYNYTDESNNKY
FGAISKTSQKSIIHYKKDSKNILEFYNDSTLEFNSKDFIAEDINVEMKDDINTITGKYYVDNNTDKSIDE
SISLVSKNQVKVNGLYLNESVYSSYLDFVKNSDGHHNTSNEMNLFLDNISFWKLFGFENINFVIDKYFTL
VGKTNLGYVEFICDNNKNIDIYFGEWKTSSSKSTIFSGNGRNVVVEPIYNPDTGEDISTSLDESYEPLYG
IDRYINKVLIAPDLYTSLININTNYYSNEYYPEIIVLNPNTFHKKVNINLDSSSFEYKWSTEGSDFILVR
YLEESNKKILQKIRIKGILSNTQSENKMSIDFKDIKKLSLGYIMSNEKSENSENELDRDHLGFKIIDNKT
YYYDEDSKLVKGLININNSLFYFDPIEFNLVTGWQTINGKKYYFDINTGAALISYKIINGKHFYENNDGV
MQLGVFKGPDGFEYFAPANTQNNNIEGQAIVYQSKELTLNGKKYYFDNDSKAVTGWRIINNEKYYENPNN
AIAAVGLQVIDNNKYYFNPDTAIISKGWQTVNGSRYYFDTDTAIAFNGYKTIDGKHFYFDSDCVVKIGVE
STSNGFEYFAPANTYNNNIEGQAIVYQSKELTLNGKKYYEDNNSKAVTGWQTIDSKKYYENTNTAEAATG
WQTIDGKKYYFNTNTAEAATGWQTIDGKKYYFNTNTAIASTGYTIINGKHFYFNTDGIMQIGVEKGPNGF
EYFAPANTDANNIEGQAILYQNEFLTLNGKKYYFGSDSKAVTGWRIINNKKYYENPNNAIAAIHLCTINN
DKYYFSYDGILQNGYITIERNNFYFDANNESKMVTGVFKGPNGFEYFAPANTHNNNIEGQAIVYQNKELT
LNGKKYYFDNDSKAVTGWQTIDGKKYYENLNTAEAATGWQTIDGKKYYENLNTAEAATGWQTIDGKKYYF
NTNTFIASTGYTSINGKHFYFNTDGIMQIGVEKGPNGFEYFAPANTHNNNIEGQAILYQNKELTLNGKKY
YFGSDSKAVTGLRTIDGKKYYFNTNTAVAVTGWQTINGKKYYFNTNTSIASTGYTIISGKHFYENTDGIM
QIGVFKGPDGFEYFAPANTDANNIEGQAIRYQNRFLYLHDNIYYFGNNSKAATGWVTIDGNRYYFEPNTA
MGANGYKTIDNKNFYFRNGLPQIGVFKGSNGFEYFAPANTDANNIEGQAIRYQNRFLHLLGKIYYFGNNS
KAVTGWQTINGKVYYFMPDTAMAAAGGLFEIDGVIYFFGVDGVKAPGIYG

SEQ ID NO: 5 sets forth the amino acid sequence for a mutant TcdB having a mutation at positions 286 and 288, as compared to SEQ ID NO: 2.
SEQ ID NO: 6 sets forth the amino acid sequence for a mutant TcdB having a mutation at positions 286, 288, and 698, as compared to SEQ ID NO: 2.

(SEQ ID NO: 6)
MSLVNRKQLEKMANVRERTQEDEYVAILDALEEYHNMSENTVVEKYLKLKDINSLTDIYIDTYKKSGRNK
ALKKFKEYLVTEVLELKNNNLTPVEKNLHFVWIGGQINDTAINYINQWKDVNSDYNVNVFYDSNAFLINT
LKKTVVESAINDTLESFRENLNDPREDYNKFFRKRMEIIYDKQKNFINYYKAQREENPELIIDDIVKTYL
SNEYSKEIDELNTYIEESLNKITQNSGNDVRNFEEFKNGESENLYEQELVERWNLAAASDILRISALKEI
GGMYLAVAMLPGIQPDLFESIEKPSSVTVDEWEMTKLEAIMKYKEYIPEYTSEHEDMLDEEVQSSFESVL
ASKSDKSEIFSSLGDMEASPLEVKIAFNSKGIINQGLISVKDSYCSNLIVKQIENRYKILNNSLNPAISE
DNDENTTTNTFIDSIMAEANADNGREMMELGKYLRVGFFPDVKTTINLSGPEAYAAAYQDLLMFKEGSMN
IHLIEADLRNFEISKTNISQSTEQEMASLWSEDDARAKAQFEEYKRNYFEGSLGEDDNLDESQNIVVDKE
YLLEKISSLARSSERGYIHYIVQLQGDKISYEAACNLFAKTPYDSVLFQKNIEDSEIAYYYNPGDGEIQE
IDKYKIPSIISDRPKIKLTFIGHGKDEFNTDIFAGFDVDSLSTEIEAAIDLAKEDISPKSIEINLLGANM
FSYSINVEETYPGKLLLKVKDKISELMPSISQDSIIVSANQYEVRINSEGRRELLDHSGEWINKEESIIK
DISSKEYISFNPKENKITVKSKNLPELSTLLQEIRNNSNSSDIELEEKVMLTECEINVISNIDTQIVEER
IEEAKNLTSDSINYIKDEFKLIESISDALCDLKQQNELEDSHFISFEDISETDEGFSIRFINKETGESIF
VETEKTIFSEYANHITEEISKIKGTIFDTVNGKLVKKVNLDTTHEVNTLNAAFFIQSLIEYNSSKESLSN
LSVAMKVQVYAQLFSTGLNTITDAAKVVELVSTALDETIDLLPTLSEGLPIIATIIDGVSLGAAIKELSE
TSDPLLRQEIEAKIGIMAVNLTTATTAIITSSLGIASGFSILLVPLAGISAGIPSLVNNELVLRDKATKV
VDYFKHVSLVETEGVFTLLDDKIMMPQDDLVISEIDENNNSIVLGKCEIWRMEGGSGHTVTDDIDHFFSA
PSITYREPHLSIYDVLEVQKEELDLSKDLMVLPNAPNRVFAWETGWTPGLRSLENDGTKLLDRIRDNYEG
EFYWRYFAFIADALITTLKPRYEDTNIRINLDSNTRSFIVPIITTEYIREKLSYSFYGSGGTYALSLSQY
NMGINIELSESDVWIIDVDNVVRDVTIESDKIKKGDLIEGILSTLSIEENKIILNSHEINESGEVNGSNG
FVSLTESILEGINAIIEVDLLSKSYKLLISGELKILMLNSNHIQQKIDYIGENSELQKNIPYSFVDSEGK
ENGFINGSTKEGLFVSELPDVVLISKVYMDDSKPSFGYYSNNLKDVKVITKDNVNILTGYYLKDDIKISL
SLTLQDEKTIKLNSVHLDESGVAEILKEMNRKGNTNTSDSLMSFLESMNIKSIFVNELQSNIKFILDANE
IISGTTSIGQFEFICDENDNIQPYFIKENTLETNYTLYVGNRQNMIVEPNYDLDDSGDISSTVINFSQKY
LYGIDSCVNKVVISPNIYTDEINITPVYETNNTYPEVIVLDANYINEKINVNINDLSIRYVWSNDGNDFI
LMSTSEENKVSQVKIRFVNVEKDKTLANKLSENFSDKQDVPVSEIILSFTPSYYEDGLIGYDLGLVSLYN
EKFYINNFGMMVSGLIYINDSLYYFKPPVNNLITGFVTVGDDKYYENPINGGAASIGETIIDDKNYYENQ
SGVLQTGVESTEDGEKYFAPANTLDENLEGEAIDETGKLIIDENTYYEDDNYRGAVEWKELDGEMHYESP
ETGKAFKGLNQIGDYKYYFNSDGVMQKGFVSINDNKHYFDDSGVMKVGYTEIDGKHFYFAENGEMQIGVE
NTEDGFKYFAHHNEDLGNEEGEEISYSGILNENNKIYYFDDSFTAVVGWKDLEDGSKYYEDEDTAEAYIG
LSLINDGQYYFNDDGIMQVGFVTINDKVFYFSDSGIIESGVQNIDDNYFYIDDNGIVQIGVEDTSDGYKY
FAPANTVNDNIYGQAVEYSGLVRVGEDVYYFGETYTIETGWIYDMENESDKYYENPETKKACKGINLIDD
IKYYFDEKGIMRTGLISFENNNYYFNENGEMQFGYINIEDKMFYFGEDGVMQIGVENTPDGFKYFAHQNT
LDENFEGESINYTGWLDLDEKRYYFTDEYIAATGSVIIDGEEYYFDPDTAQLVISE

SEQ ID NO: 7 sets forth the amino acid sequence for a mutant TcdA having a mutation at positions 269, 272, 285, 287, 460, 462, and 700, as compared to SEQ ID NO: 1
SEQ ID NO: 8 sets forth the amino acid sequence for a mutant TcdB having a mutation at positions 270, 273, 286, 288, 461, 463, and 698, as compared to SEQ ID NO: 2
SEQ ID NO: 9 sets forth a DNA sequence encoding a wild-type C. difficile 630 toxin A (TcdA).
SEQ ID NO: 10 sets forth a DNA sequence encoding a wild-type C. difficile 630 toxin B (TcdB).
SEQ ID NO: 11 sets forth a DNA sequence encoding SEQ ID NO: 3
SEQ ID NO: 12 sets forth a DNA sequence encoding SEQ ID NO: 4
SEQ ID NO: 13 sets forth a DNA sequence encoding SEQ ID NO: 5
SEQ ID NO: 14 sets forth a DNA sequence encoding SEQ ID NO: 6
SEQ ID NO: 15 sets forth the amino acid sequence for wild-type C. difficile R20291 TcdA.
SEQ ID NO: 16 sets forth a DNA sequence encoding SEQ ID NO: 15.
SEQ ID NO: 17 sets forth the amino acid sequence for wild-type C. difficile CD196 TcdA.
SEQ ID NO: 18 sets forth a DNA sequence encoding SEQ ID NO: 17.
SEQ ID NO: 19 sets forth the amino acid sequence for wild-type C. difficile VPI10463 TcdA.
SEQ ID NO: 20 sets forth a DNA sequence encoding SEQ ID NO: 19.
SEQ ID NO: 21 sets forth the amino acid sequence for wild-type C. difficile R20291 TcdB.
SEQ ID NO: 22 sets forth a DNA sequence encoding SEQ ID NO: 21.
SEQ ID NO: 23 sets forth the amino acid sequence for wild-type C. difficile CD196 TcdB.
SEQ ID NO: 24 sets forth a DNA sequence encoding SEQ ID NO: 23.
SEQ ID NO: 25 sets forth the amino acid sequence for wild-type C. difficile VPI10463 TcdB.
SEQ ID NO: 26 sets forth a DNA sequence encoding SEQ ID NO: 25.
SEQ ID NO: 27 sets forth a DNA sequence of a pathogenicity locus of wild-type C. difficile VPI10463.
SEQ ID NO: 28 sets forth the amino acid sequence for residues 101 to 293 of SEQ ID NO: 1.
SEQ ID NO: 29 sets forth the amino acid sequence for residues 1 to 542 of SEQ ID NO: 1.
SEQ ID NO: 30 sets forth the amino acid sequence for residues 101 to 293 of SEQ ID NO: 2.
SEQ ID NO: 31 sets forth the amino acid sequence for residues 1 to 543 of SEQ ID NO: 2.
SEQ ID NO: 32 sets forth the amino acid sequence for residues 543 to 809 of SEQ ID NO: 1.
SEQ ID NO: 33 sets forth the amino acid sequence for residues 544 to 767 of SEQ ID NO: 2.
SEQ ID NO: 34 sets forth the amino acid sequence for a mutant TcdA, wherein residues 101, 269, 272, 285, 287, 460, 462, 541, 542, 543, 589, 655, and 700 may be any amino acid.
SEQ ID NO: 35 sets forth the amino acid sequence for a mutant TcdB, wherein 102, 270, 273, 286, 288, 384, 461, 463, 520, 543, 544, 587, 600, 653, 698, and 751 may be any amino acid.
SEQ ID NO: 36 sets forth the amino acid sequence for the variable light chain of a neutralizing antibody of C. difficile TcdA (A3-25 mAb).
SEQ ID NO: 37 sets forth the amino acid sequence for the variable heavy chain of a neutralizing antibody of C. difficile TcdA (A3-25 mAb).
SEQ ID NO: 38 sets forth the amino acid sequence for CDR1 of the variable light chain of neutralizing antibody of C. difficile TcdA (A3-25 mAb).
SEQ ID NO: 39 sets forth the amino acid sequence for CDR2 of the variable light chain of neutralizing antibody of C. difficile TcdA (A3-25 mAb).
SEQ ID NO: 40 sets forth the amino acid sequence for CDR3 of the variable light chain of neutralizing antibody of C. difficile TcdA (A3-25 mAb).
SEQ ID NO: 41 sets forth the amino acid sequence for CDR1 of the variable heavy chain of neutralizing antibody of C. difficile TcdA (A3-25 mAb).
SEQ ID NO: 42 sets forth the amino acid sequence for CDR2 of the variable heavy chain of neutralizing antibody of C. difficile TcdA (A3-25 mAb).
SEQ ID NO: 43 sets forth the amino acid sequence for CDR3 of the variable heavy chain of neutralizing antibody of C. difficile TcdA (A3-25 mAb).
SEQ ID NO: 44 sets forth a DNA sequence encoding SEQ ID NO: 3.
SEQ ID NO: 45 sets forth a DNA sequence encoding SEQ ID NO: 4.
SEQ ID NO: 46 sets forth a DNA sequence encoding SEQ ID NO: 5.
SEQ ID NO: 47 sets forth a DNA sequence encoding SEQ ID NO: 6.
SEQ ID NO: 48 sets forth the nucleotide sequence of immunostimulatory oligonucleotide ODN CpG 24555.
SEQ ID NO: 49 sets forth the amino acid sequence for the variable heavy chain of a C. difficile TcdB neutralizing antibody (B8-26 mAb).
SEQ ID NO: 50 sets forth the amino acid sequence for the signal peptide of the variable heavy chain of a C. difficile TcdB neutralizing antibody (B8-26 mAb).
SEQ ID NO: 51 sets forth the amino acid sequence for CDR1 of the variable heavy chain of a C. difficile TcdB neutralizing antibody (B8-26 mAb).
SEQ ID NO: 52 sets forth the amino acid sequence for CDR2 of the variable heavy chain of a C. difficile TcdB neutralizing antibody (B8-26 mAb).
SEQ ID NO: 53 sets forth the amino acid sequence for CDR3 of the variable heavy chain of a C. difficile TcdB neutralizing antibody (B8-26 mAb).
SEQ ID NO: 54 sets forth the amino acid sequence for the constant region of the variable heavy chain of a C. difficile TcdB neutralizing antibody (B8-26 mAb).
SEQ ID NO: 55 sets forth the amino acid sequence for the variable light chain of a C. difficile TcdB neutralizing antibody (B8-26 mAb).
SEQ ID NO: 56 sets forth the amino acid sequence for the signal peptide of the variable light chain of a C. difficile TcdB neutralizing antibody (B8-26 mAb).
SEQ ID NO: 57 sets forth the amino acid sequence for CDR1 of the variable light chain of a C. difficile TcdB neutralizing antibody (B8-26 mAb).
SEQ ID NO: 58 sets forth the amino acid sequence for CDR2 of the variable light chain of a C. difficile TcdB neutralizing antibody (B8-26 mAb).
SEQ ID NO: 59 sets forth the amino acid sequence for CDR3 of the variable light chain of a C. difficile TcdB neutralizing antibody (B8-26 mAb).
SEQ ID NO: 60 sets forth the amino acid sequence for the variable heavy chain of a C. difficile TcdB neutralizing antibody (B59-3 mAb).
SEQ ID NO: 61 sets forth the amino acid sequence for the signal peptide of the variable heavy chain of a C. difficile TcdB neutralizing antibody (B59-3 mAb).
SEQ ID NO: 62 sets forth the amino acid sequence for CDR1 of the variable heavy chain of a C. difficile TcdB neutralizing antibody (B59-3 mAb).
SEQ ID NO: 63 sets forth the amino acid sequence for CDR2 of the variable heavy chain of a C. difficile TcdB neutralizing antibody (B59-3 mAb).
SEQ ID NO: 64 sets forth the amino acid sequence for CDR3 of the variable heavy chain of a C. difficile TcdB neutralizing antibody (B59-3 mAb).
SEQ ID NO: 65 sets forth the amino acid sequence for the constant region of the variable heavy chain of a C. difficile TcdB neutralizing antibody (B59-3 mAb).
SEQ ID NO: 66 sets forth the amino acid sequence for the variable light chain of a C. difficile TcdB neutralizing antibody (B59-3 mAb).
SEQ ID NO: 67 sets forth the amino acid sequence for the signal peptide of the variable light chain of a C. difficile TcdB neutralizing antibody (B59-3 mAb).
SEQ ID NO: 68 sets forth the amino acid sequence for CDR1 of the variable light chain of a C. difficile TcdB neutralizing antibody (B59-3 mAb).
SEQ ID NO: 69 sets forth the amino acid sequence for CDR2 of the variable light chain of a C. difficile TcdB neutralizing antibody (B59-3 mAb).
SEQ ID NO: 70 sets forth the amino acid sequence for CDR3 of the variable light chain of a C. difficile TcdB neutralizing antibody (B59-3 mAb).
SEQ ID NO: 71 sets forth the amino acid sequence for the variable heavy chain of a C. difficile TcdB neutralizing antibody (B9-30 mAb).
SEQ ID NO: 72 sets forth the amino acid sequence for the signal peptide of the variable heavy chain of a C. difficile TcdB neutralizing antibody (B9-30 mAb).
SEQ ID NO: 73 sets forth the amino acid sequence for CDR1 of the variable heavy chain of a C. difficile TcdB neutralizing antibody (B9-30 mAb).
SEQ ID NO: 74 sets forth the amino acid sequence for CDR2 of the variable heavy chain of a C. difficile TcdB neutralizing antibody (B9-30 mAb).
SEQ ID NO: 75 sets forth the amino acid sequence for CDR3 of the variable heavy chain of a C. difficile TcdB neutralizing antibody (B9-30 mAb).
SEQ ID NO: 76 sets forth the amino acid sequence for the constant region of the variable heavy chain of a C. difficile TcdB neutralizing antibody (B9-30 mAb).
SEQ ID NO: 77 sets forth the amino acid sequence for the variable light chain of a C. difficile TcdB neutralizing antibody (B9-30 mAb).
SEQ ID NO: 78 sets forth the amino acid sequence for the signal peptide of the variable light chain of a C. difficile TcdB neutralizing antibody (B9-30 mAb).
SEQ ID NO: 79 sets forth the amino acid sequence for CDR1 of the variable light chain of a C. difficile TcdB neutralizing antibody (B9-30 mAb).
SEQ ID NO: 80 sets forth the amino acid sequence for CDR2 of the variable light chain of a C. difficile TcdB neutralizing antibody (B9-30 mAb).
SEQ ID NO: 81 sets forth the amino acid sequence for CDR3 of the variable light chain of a C. difficile TcdB neutralizing antibody (B9-30 mAb).
SEQ ID NO: 82 sets forth the amino acid sequence for a mutant TcdB, wherein a residue at positions 102, 270, 273, 286, 288, 384, 461, 463, 520, 543, 544, 587, 600, 653, 698, and 751 may be any amino acid.
SEQ ID NO: 83 sets forth the amino acid sequence for a mutant TcdA having a mutation at positions 269, 272, 285, 287, 460, 462, and 700, as compared to SEQ ID NO: 1, wherein the methionine at position 1 is absent.
SEQ ID NO: 84 sets forth the amino acid sequence for a mutant C. difficile toxin A having a mutation at positions 285, 287, and 700, as compared to SEQ ID NO: 1, wherein the methionine at position 1 is absent.

(SEQ ID NO: 84)
SLISKEELIKLAYSIRPRENEYKTILTNLDEYNKLTINNNENKYLQLKKLNESIDVEMNKYKTSSRNRAL
SNLKKDILKEVILIKNSNTSPVEKNLHFVWIGGEVSDIALEYIKQWADINAEYNIKLWYDSEAFLVNTLK
KAIVESSTTEALQLLEEEIQNPQFDNMKFYKKRMEFIYDRQKRFINYYKSQINKPTVPTIDDIIKSHLVS
EYNRDETVLESYRTNSLRKINSNHGIDIRANSLFTEQELLNIYSQELLNRGNLAAASDIVRLLALKNFGG
VYLAVAMLPGIHSDLFKTISRPSSIGLDRWEMIKLEAIMKYKKYINNYTSENFDKLDQQLKDNFKLIIES
KSEKSEIFSKLENLNVSDLEIKIAFALGSVINQALISKQGSYLTNLVIEQVKNRYQFLNQHLNPAIESDN
NFTDTTKIFHDSLENSATAENSMELTKIAPYLQVGFMPEARSTISLSGPGAYASAYYDFINLQENTIEKT
LKASDLIEFKEPENNLSQLTEQEINSLWSFDQASAKYQFEKYVRDYTGGSLSEDNGVDENKNTALDKNYL
LNNKIPSNNVEEAGSKNYVHYIIQLQGDDISYEATCNLESKNPKNSIIIQRNMNESAKSYFLSDDGESIL
ELNKYRIPERLKNKEKVKVTFIGHGKDEENTSEFARLSVDSLSNEISSELDTIKLDISPKNVEVNLLGAN
MFSYDENVEETYPGKLLLSIMDKITSTLPDVNKNSITIGANQYEVRINSEGRKELLAHSGKWINKEEAIM
SDLSSKEYIFFDSIDNKLKAKSKNIPGLASISEDIKTLLLDASVSPDTKFILNNLKLNIESSIGDYIYYE
KLEPVKNIIHNSIDDLIDEFNLLENVSDELYELKKLNNLDEKYLISFEDISKNNSTYSVRFINKSNGESV
YVETEKEIFSKYSEHITKEISTIKNSIITDVNGNLLDNIQLDHTSQVNTLNAAFFIQSLIDYSSNKDVLN
DLSTSVKVQLYAQLFSTGLNTIYDSIQLVNLISNAVNDTINVLPTITEGIPIVSTILDGINLGAAIKELL
DEHDPLLKKELEAKVGVLAINMSLSIAATVASIVGIGAEVTIELLPIAGISAGIPSLVNNELILHDKATS
VVNYFNHLSESKKYGPLKTEDDKILVPIDDLVISEIDENNNSIKLGTCNILAMEGGSGHTVTGNIDHFFS
SPSISSHIPSLSIYSAIGIETENLDESKKIMMLPNAPSRVEWWETGAVPGLRSLENDGTRLLDSIRDLYP
GKFYWRFYAFFDYAITTLKPVYEDTNIKIKLDKDTRNFIMPTITTNEIRNKLSYSFDGAGGTYSLLLSSY
PISTNINLSKDDLWIENIDNEVREISIENGTIKKGKLIKDVLSKIDINKNKLIIGNQTIDESGDIDNKDR
YIFLTCELDDKISLIIEINLVAKSYSLLLSGDKNYLISNLSNIIEKINTLGLDSKNIAYNYTDESNNKYF
GAISKTSQKSIIHYKKDSKNILEFYNDSTLEFNSKDFIAEDINVEMKDDINTITGKYYVDNNTDKSIDES
ISLVSKNQVKVNGLYLNESVYSSYLDFVKNSDGHHNTSNEMNLELDNISEWKLEGFENINFVIDKYFTLV
GKTNLGYVEFICDNNKNIDIYFGEWKTSSSKSTIFSGNGRNVVVEPIYNPDTGEDISTSLDESYEPLYGI
DRYINKVLIAPDLYTSLININTNYYSNEYYPEIIVLNPNTFHKKVNINLDSSSFEYKWSTEGSDFILVRY
LEESNKKILQKIRIKGILSNTQSFNKMSIDFKDIKKLSLGYIMSNFKSENSENELDRDHLGFKIIDNKTY
YYDEDSKLVKGLININNSLFYFDPIEFNLVTGWQTINGKKYYFDINTGAALISYKIINGKHFYENNDGVM
QLGVFKGPDGFEYFAPANTQNNNIEGQAIVYQSKELTLNGKKYYFDNDSKAVTGWRIINNEKYYENPNNA
IAAVGLQVIDNNKYYFNPDTAIISKGWQTVNGSRYYFDTDTAIAFNGYKTIDGKHFYFDSDCVVKIGVES
TSNGFEYFAPANTYNNNIEGQAIVYQSKELTLNGKKYYFDNNSKAVTGWQTIDSKKYYENTNTAEAATGW
QTIDGKKYYFNTNTAEAATGWQTIDGKKYYENTNTAIASTGYTIINGKHFYFNTDGIMQIGVEKGPNGFE
YFAPANTDANNIEGQAILYQNEFLTLNGKKYYFGSDSKAVTGWRIINNKKYYENPNNAIAAIHLCTINND
KYYFSYDGILQNGYITIERNNFYFDANNESKMVTGVFKGPNGFEYFAPANTHNNNIEGQAIVYQNKELTL
NGKKYYFDNDSKAVTGWQTIDGKKYYENLNTAEAATGWQTIDGKKYYENLNTAEAATGWQTIDGKKYYEN
TNTFIASTGYTSINGKHFYFNTDGIMQIGVFKGPNGFEYFAPANTHNNNIEGQAILYQNKELTLNGKKYY
FGSDSKAVTGLRTIDGKKYYFNTNTAVAVTGWQTINGKKYYENTNTSIASTGYTIISGKHFYENTDGIMQ
IGVFKGPDGFEYFAPANTDANNIEGQAIRYQNRFLYLHDNIYYFGNNSKAATGWVTIDGNRYYFEPNTAM
GANGYKTIDNKNFYFRNGLPQIGVFKGSNGFEYFAPANTDANNIEGQAIRYQNRFLHLLGKIYYFGNNSK
AVTGWQTINGKVYYFMPDTAMAAAGGLFEIDGVIYFFGVDGVKAPGIYG

SEQ ID NO: 85 sets forth the amino acid sequence for a mutant C. difficile toxin B having a mutation at positions 270, 273, 286, 288, 461, 463, and 698, as compared to SEQ ID NO: 2, wherein the methionine at position 1 is absent.
SEQ ID NO: 86 sets forth the amino acid sequence for a mutant C. difficile toxin B having a mutation at positions 286, 288, and 698, as compared to SEQ ID NO: 2, wherein the methionine at position 1 is absent.

(SEQ ID NO: 86)
SLVNRKQLEKMANVRFRTQEDEYVAILDALEEYHNMSENTVVEKYLKLKDINSLTDIYIDTYKKSGRNKA
LKKFKEYLVTEVLELKNNNLTPVEKNLHFVWIGGQINDTAINYINQWKDVNSDYNVNVFYDSNAFLINTL
KKTVVESAINDTLESFRENLNDPRFDYNKFFRKRMEIIYDKQKNFINYYKAQREENPELIIDDIVKTYLS
NEYSKEIDELNTYIEESLNKITQNSGNDVRNFEEFKNGESFNLYEQELVERWNLAAASDILRISALKEIG
GMYLAVAMLPGIQPDLFESIEKPSSVTVDFWEMTKLEAIMKYKEYIPEYTSEHFDMLDEEVQSSFESVLA
SKSDKSEIFSSLGDMEASPLEVKIAFNSKGIINQGLISVKDSYCSNLIVKQIENRYKILNNSLNPAISED
NDFNTTTNTFIDSIMAEANADNGRFMMELGKYLRVGFFPDVKTTINLSGPEAYAAAYQDLLMFKEGSMNI
HLIEADLRNFEISKTNISQSTEQEMASLWSEDDARAKAQFEEYKRNYFEGSLGEDDNLDESQNIVVDKEY
LLEKISSLARSSERGYIHYIVQLQGDKISYEAACNLFAKTPYDSVLFQKNIEDSEIAYYYNPGDGEIQEI
DKYKIPSIISDRPKIKLTFIGHGKDEFNTDIFAGFDVDSLSTEIEAAIDLAKEDISPKSIEINLLGANMF
SYSINVEETYPGKLLLKVKDKISELMPSISQDSIIVSANQYEVRINSEGRRELLDHSGEWINKEESIIKD
ISSKEYISFNPKFNKITVKSKNLPELSTLLQEIRNNSNSSDIELEEKVMLTECEINVISNIDTQIVEERI
EEAKNLTSDSINYIKDEFKLIESISDALCDLKQQNELEDSHFISFEDISETDEGFSIRFINKETGESIFV
ETEKTIFSEYANHITEEISKIKGTIFDTVNGKLVKKVNLDTTHEVNTLNAAFFIQSLIEYNSSKESLSNL
SVAMKVQVYAQLFSTGLNTITDAAKVVELVSTALDETIDLLPTLSEGLPIIATIIDGVSLGAAIKELSET
SDPLLRQEIEAKIGIMAVNLTTATTAIITSSLGIASGESILLVPLAGISAGIPSLVNNELVLRDKATKVV
DYFKHVSLVETEGVFTLLDDKIMMPQDDLVISEIDENNNSIVLGKCEIWRMEGGSGHTVTDDIDHFFSAP
SITYREPHLSIYDVLEVQKEELDLSKDLMVLPNAPNRVFAWETGWTPGLRSLENDGTKLLDRIRDNYEGE
FYWRYFAFIADALITTLKPRYEDTNIRINLDSNTRSFIVPIITTEYIREKLSYSFYGSGGTYALSLSQYN
MGINIELSESDVWIIDVDNVVRDVTIESDKIKKGDLIEGILSTLSIEENKIILNSHEINESGEVNGSNGF
VSLTFSILEGINAIIEVDLLSKSYKLLISGELKILMLNSNHIQQKIDYIGFNSELQKNIPYSFVDSEGKE
NGFINGSTKEGLFVSELPDVVLISKVYMDDSKPSFGYYSNNLKDVKVITKDNVNILTGYYLKDDIKISLS
LTLQDEKTIKLNSVHLDESGVAEILKFMNRKGNTNTSDSLMSFLESMNIKSIFVNFLQSNIKFILDANFI
ISGTTSIGQFEFICDENDNIQPYFIKFNTLETNYTLYVGNRQNMIVEPNYDLDDSGDISSTVINFSQKYL
YGIDSCVNKVVISPNIYTDEINITPVYETNNTYPEVIVLDANYINEKINVNINDLSIRYVWSNDGNDFIL
MSTSEENKVSQVKIRFVNVFKDKTLANKLSFNFSDKQDVPVSEIILSFTPSYYEDGLIGYDLGLVSLYNE
KFYINNFGMMVSGLIYINDSLYYFKPPVNNLITGFVTVGDDKYYFNPINGGAASIGETIIDDKNYYFNQS
GVLQTGVFSTEDGFKYFAPANTLDENLEGEAIDFTGKLIIDENIYYFDDNYRGAVEWKELDGEMHYFSPE
TGKAFKGLNQIGDYKYYFNSDGVMQKGFVSINDNKHYFDDSGVMKVGYTEIDGKHFYFAENGEMQIGVFN
TEDGFKYFAHHNEDLGNEEGEEISYSGILNFNNKIYYFDDSFTAVVGWKDLEDGSKYYEDEDTAEAYIGL
SLINDGQYYFNDDGIMQVGFVTINDKVFYFSDSGIIESGVQNIDDNYFYIDDNGIVQIGVEDTSDGYKYF
APANTVNDNIYGQAVEYSGLVRVGEDVYYFGETYTIETGWIYDMENESDKYYFNPETKKACKGINLIDDI
KYYFDEKGIMRTGLISFENNNYYFNENGEMQFGYINIEDKMFYFGEDGVMQIGVFNTPDGFKYFAHQNTL
DENFEGESINYTGWLDLDEKRYYFTDEYIAATGSVIIDGEEYYFDPDTAQLVISE

SEQ ID NO: 87 sets forth the amino acid sequence for wild-type C. difficile 2004013 TcdA.
SEQ ID NO: 88 sets forth the amino acid sequence for wild-type C. difficile 2004111 TcdA.
SEQ ID NO: 89 sets forth the amino acid sequence for wild-type C. difficile 2004118 TcdA.
SEQ ID NO: 90 sets forth the amino acid sequence for wild-type C. difficile 2004205 TcdA.
SEQ ID NO: 91 sets forth the amino acid sequence for wild-type C. difficile 2004206 TcdA.
SEQ ID NO: 92 sets forth the amino acid sequence for wild-type C. difficile 2005022 TcdA.
SEQ ID NO: 93 sets forth the amino acid sequence for wild-type C. difficile 2005088 TcdA.
SEQ ID NO: 94 sets forth the amino acid sequence for wild-type C. difficile 2005283 TcdA.
SEQ ID NO: 95 sets forth the amino acid sequence for wild-type C. difficile 2005325 TcdA.
SEQ ID NO: 96 sets forth the amino acid sequence for wild-type C. difficile 2005359 TcdA.
SEQ ID NO: 97 sets forth the amino acid sequence for wild-type C. difficile 2006017 TcdA.
SEQ ID NO: 98 sets forth the amino acid sequence for wild-type C. difficile 2007070 TcdA.
SEQ ID NO: 99 sets forth the amino acid sequence for wild-type C. difficile 2007217 TcdA.
SEQ ID NO: 100 sets forth the amino acid sequence for wild-type C. difficile 2007302 TcdA.
SEQ ID NO: 101 sets forth the amino acid sequence for wild-type C. difficile 2007816 TcdA.
SEQ ID NO: 102 sets forth the amino acid sequence for wild-type C. difficile 2007838 TcdA.
SEQ ID NO: 103 sets forth the amino acid sequence for wild-type C. difficile 2007858 TcdA.
SEQ ID NO: 104 sets forth the amino acid sequence for wild-type C. difficile 2007886 TcdA.
SEQ ID NO: 105 sets forth the amino acid sequence for wild-type C. difficile 2008222 TcdA.
SEQ ID NO: 106 sets forth the amino acid sequence for wild-type C. difficile 2009078 TcdA.
SEQ ID NO: 107 sets forth the amino acid sequence for wild-type C. difficile 2009087 TcdA.
SEQ ID NO: 108 sets forth the amino acid sequence for wild-type C. difficile 2009141 TcdA.
SEQ ID NO: 109 sets forth the amino acid sequence for wild-type C. difficile 2009292 TcdA.
SEQ ID NO: 110 sets forth the amino acid sequence for wild-type C. difficile 2004013 TcdB.
SEQ ID NO: 111 sets forth the amino acid sequence for wild-type C. difficile 2004111 TcdB.
SEQ ID NO: 112 sets forth the amino acid sequence for wild-type C. difficile 2004118 TcdB.
SEQ ID NO: 113 sets forth the amino acid sequence for wild-type C. difficile 2004205 TcdB.
SEQ ID NO: 114 sets forth the amino acid sequence for wild-type C. difficile 2004206 TcdB.
SEQ ID NO: 115 sets forth the amino acid sequence for wild-type C. difficile 2005022 TcdB.
SEQ ID NO: 116 sets forth the amino acid sequence for wild-type C. difficile 2005088 TcdB.
SEQ ID NO: 117 sets forth the amino acid sequence for wild-type C. difficile 2005283 TcdB.
SEQ ID NO: 118 sets forth the amino acid sequence for wild-type C. difficile 2005325 TcdB.
SEQ ID NO: 119 sets forth the amino acid sequence for wild-type C. difficile 2005359 TcdB.
SEQ ID NO: 120 sets forth the amino acid sequence for wild-type C. difficile 2006017 TcdB.
SEQ ID NO: 121 sets forth the amino acid sequence for wild-type C. difficile 2006376 TcdB.
SEQ ID NO: 122 sets forth the amino acid sequence for wild-type C. difficile 2007070 TcdB.
SEQ ID NO: 123 sets forth the amino acid sequence for wild-type C. difficile 2007217 TcdB.
SEQ ID NO: 124 sets forth the amino acid sequence for wild-type C. difficile 2007302 TcdB.
SEQ ID NO: 125 sets forth the amino acid sequence for wild-type C. difficile 2007816 TcdB.
SEQ ID NO: 126 sets forth the amino acid sequence for wild-type C. difficile 2007838 TcdB.
SEQ ID NO: 127 sets forth the amino acid sequence for wild-type C. difficile 2007858 TcdB.
SEQ ID NO: 128 sets forth the amino acid sequence for wild-type C. difficile 2007886 TcdB.
SEQ ID NO: 129 sets forth the amino acid sequence for wild-type C. difficile 2008222 TcdB.
SEQ ID NO: 130 sets forth the amino acid sequence for wild-type C. difficile 2009078 TcdB.
SEQ ID NO: 131 sets forth the amino acid sequence for wild-type C. difficile 2009087 TcdB.
SEQ ID NO: 132 sets forth the amino acid sequence for wild-type C. difficile 2009141 TcdB.
SEQ ID NO: 133 sets forth the amino acid sequence for wild-type C. difficile 2009292 TcdB.
SEQ ID NO: 134 sets forth the amino acid sequence for wild-type C. difficile 014 TcdA.
SEQ ID NO: 135 sets forth the amino acid sequence for wild-type C. difficile 015 TcdA.
SEQ ID NO: 136 sets forth the amino acid sequence for wild-type C. difficile 020 TcdA.
SEQ ID NO: 137 sets forth the amino acid sequence for wild-type C. difficile 023 TcdA.
SEQ ID NO: 138 sets forth the amino acid sequence for wild-type C. difficile 027 TcdA.
SEQ ID NO: 139 sets forth the amino acid sequence for wild-type C. difficile 029 TcdA.
SEQ ID NO: 140 sets forth the amino acid sequence for wild-type C. difficile 046 TcdA.
SEQ ID NO: 141 sets forth the amino acid sequence for wild-type C. difficile 014 TcdB.
SEQ ID NO: 142 sets forth the amino acid sequence for wild-type C. difficile 015 TcdB.
SEQ ID NO: 143 sets forth the amino acid sequence for wild-type C. difficile 020 TcdB.
SEQ ID NO: 144 sets forth the amino acid sequence for wild-type C. difficile 023 TcdB.
SEQ ID NO: 145 sets forth the amino acid sequence for wild-type C. difficile 027 TcdB.
SEQ ID NO: 146 sets forth the amino acid sequence for wild-type C. difficile 029 TcdB.
SEQ ID NO: 147 sets forth the amino acid sequence for wild-type C. difficile 046 TcdB.
SEQ ID NO: 148 sets forth the amino acid sequence for wild-type C. difficile 001 TcdA.
SEQ ID NO: 149 sets forth the amino acid sequence for wild-type C. difficile 002 TcdA.
SEQ ID NO: 150 sets forth the amino acid sequence for wild-type C. difficile 003 TcdA.
SEQ ID NO: 151 sets forth the amino acid sequence for wild-type C. difficile 004 TcdA.
SEQ ID NO: 152 sets forth the amino acid sequence for wild-type C. difficile 070 TcdA.
SEQ ID NO: 153 sets forth the amino acid sequence for wild-type C. difficile 075 TcdA.
SEQ ID NO: 154 sets forth the amino acid sequence for wild-type C. difficile 077 TcdA.
SEQ ID NO: 155 sets forth the amino acid sequence for wild-type C. difficile 081 TcdA.
SEQ ID NO: 156 sets forth the amino acid sequence for wild-type C. difficile 117 TcdA.
SEQ ID NO: 157 sets forth the amino acid sequence for wild-type C. difficile 131 TcdA.
SEQ ID NO: 158 sets forth the amino acid sequence for wild-type C. difficile 001 TcdB.
SEQ ID NO: 159 sets forth the amino acid sequence for wild-type C. difficile 002 TcdB.
SEQ ID NO: 160 sets forth the amino acid sequence for wild-type C. difficile 003 TcdB.
SEQ ID NO: 161 sets forth the amino acid sequence for wild-type C. difficile 004 TcdB.
SEQ ID NO: 162 sets forth the amino acid sequence for wild-type C. difficile 070 TcdB.
SEQ ID NO: 163 sets forth the amino acid sequence for wild-type C. difficile 075 TcdB.
SEQ ID NO: 164 sets forth the amino acid sequence for wild-type C. difficile 077 TcdB.
SEQ ID NO: 165 sets forth the amino acid sequence for wild-type C. difficile 081 TcdB.
SEQ ID NO: 166 sets forth the amino acid sequence for wild-type C. difficile 117 TcdB.
SEQ ID NO: 167 sets forth the amino acid sequence for wild-type C. difficile 131 TcdB.
SEQ ID NO: 168 sets forth the amino acid sequence for wild-type C. difficile 053 TcdA.
SEQ ID NO: 169 sets forth the amino acid sequence for wild-type C. difficile 078 TcdA.
SEQ ID NO: 170 sets forth the amino acid sequence for wild-type C. difficile 087 TcdA.
SEQ ID NO: 171 sets forth the amino acid sequence for wild-type C. difficile 095 TcdA.
SEQ ID NO: 172 sets forth the amino acid sequence for wild-type C. difficile 126 TcdA.
SEQ ID NO: 173 sets forth the amino acid sequence for wild-type C. difficile 053 TcdB.
SEQ ID NO: 174 sets forth the amino acid sequence for wild-type C. difficile 078 TcdB.
SEQ ID NO: 175 sets forth the amino acid sequence for wild-type C. difficile 087 TcdB.
SEQ ID NO: 176 sets forth the amino acid sequence for wild-type C. difficile 095 TcdB.
SEQ ID NO: 177 sets forth the amino acid sequence for wild-type C. difficile 126 TcdB.
SEQ ID NO: 178 sets forth the amino acid sequence for wild-type C. difficile 059 TcdA.
SEQ ID NO: 179 sets forth the amino acid sequence for wild-type C. difficile 059 TcdB.
SEQ ID NO: 180 sets forth the amino acid sequence for wild-type C. difficile 106 TcdA.
SEQ ID NO: 181 sets forth the amino acid sequence for wild-type C. difficile 106 TcdB.
SEQ ID NO: 182 sets forth the amino acid sequence for wild-type C. difficile 017 TcdB.
SEQ ID NO: 183 sets forth the amino acid sequence for a mutant TcdA having a mutation at positions 285, 287, 700, 972, and 978 as compared to SEQ ID NO: 1.
SEQ ID NO: 184 sets forth the amino acid sequence for a mutant TcdB having a mutation at positions 286, 288, 698, 970, and 976 as compared to SEQ ID NO: 2.
SEQ ID NO: 185 through SEQ ID NO: 195 each set forth the amino acid sequence for an exemplary mutant toxin.
SEQ ID NO: 196 through SEQ ID NO: 212 each set forth the amino acid sequence for an exemplary mutant toxin A.
SEQ ID NO: 213 through SEQ ID NO: 222 each set forth the amino acid sequence for an exemplary mutant toxin B.
SEQ ID NO: 223 through SEQ ID NO: 236 each set forth the amino acid sequence for an exemplary mutant toxin A.
SEQ ID NO: 237 through SEQ ID NO: 243 each set forth the amino acid sequence for an exemplary mutant toxin B.
SEQ ID NO: 244 through SEQ ID NO: 245 each set forth the amino acid sequence for an exemplary mutant toxin A.
SEQ ID NO: 246 through SEQ ID NO: 249 each set forth the amino acid sequence for an exemplary mutant toxin B.
SEQ ID NO: 250 through SEQ ID NO: 253 each set forth the amino acid sequence for an exemplary mutant toxin A.
SEQ ID NO: 254 sets forth the amino acid sequence for an exemplary mutant toxin.
SEQ ID NO: 255 through SEQ ID NO: 263 each set forth the amino acid sequence for an exemplary mutant toxin A.
SEQ ID NO: 264 through SEQ ID NO: 269 each set forth the amino acid sequence for an exemplary mutant toxin B.
SEQ ID NO: 270 through SEQ ID NO: 275 each set forth the amino acid sequence for an exemplary mutant toxin.
SEQ ID NO: 276 through SEQ ID NO: 323 each set forth the amino acid sequence for an exemplary mutant toxin A.
SEQ ID NO: 324 through SEQ ID NO: 373 each set forth the amino acid sequence for an exemplary mutant toxin B.
SEQ ID NO: 374 through SEQ ID NO: 421 each set forth the amino acid sequence for an exemplary mutant toxin A.
SEQ ID NO: 422 through SEQ ID NO: 471 each set forth the amino acid sequence for an exemplary mutant toxin B.
SEQ ID NO: 472 through SEQ ID NO: 519 each set forth the amino acid sequence for an exemplary mutant toxin A.
SEQ ID NO: 568 through SEQ ID NO: 615 each set forth the amino acid sequence for an exemplary mutant toxin B.
SEQ ID NO: 520 through SEQ ID NO: 567 each set forth the amino acid sequence for an exemplary mutant toxin A.
SEQ ID NO: 616 through SEQ ID NO: 663 each set forth the amino acid sequence for an exemplary mutant toxin B.
SEQ ID NO: 664 through SEQ ID NO: 711 each set forth the amino acid sequence for an exemplary mutant toxin A.
SEQ ID NO: 712 through SEQ ID NO: 761 each set forth the amino acid sequence for an exemplary mutant toxin B.
SEQ ID NO: 762 through SEQ ID NO: 800 each set forth the amino acid sequence for a toxin A variant.
SEQ ID NO: 801 through SEQ ID NO: 840 each set forth the amino acid sequence for a toxin B variant.
SEQ ID NO: 841 sets forth the nucleotide sequence of a B class oligonucleotide (CpG 1018).
SEQ ID NO: 842 sets forth the nucleotide sequence of a B class oligonucleotide (CpG 7909).
SEQ ID NO: 843 sets forth the nucleotide sequence of a B class oligonucleotide (CpG 10103).
SEQ ID NO: 844 sets forth the nucleotide sequence of a B class oligonucleotide (CpG 1826).
SEQ ID NO: 845 sets forth the nucleotide sequence of a B class oligonucleotide.
SEQ ID NO: 846 sets forth the nucleotide sequence of a B class oligonucleotide.
SEQ ID NO: 847 sets forth the nucleotide sequence of a class oligonucleotide.
SEQ ID NO: 848 sets forth the nucleotide sequence of a C class oligonucleotide.
SEQ ID NO: 849 sets forth the nucleotide sequence of a C class oligonucleotide.
SEQ ID NO: 850 sets forth the nucleotide sequence of a C class oligonucleotide.
SEQ ID NO: 851 sets forth the nucleotide sequence of a C class oligonucleotide.
SEQ ID NO: 852 sets forth the nucleotide sequence of a C class oligonucleotide.
SEQ ID NO: 853 sets forth the nucleotide sequence of a C class oligonucleotide.
SEQ ID NO: 854 sets forth the nucleotide sequence of a C class oligonucleotide.
SEQ ID NO: 855 sets forth the nucleotide sequence of a C class oligonucleotide.
SEQ ID NO: 856 sets forth the nucleotide sequence of a C class oligonucleotide.
SEQ ID NO: 857 sets forth the nucleotide sequence of a C class oligonucleotide.
SEQ ID NO: 858 sets forth the nucleotide sequence of a C class oligonucleotide.
SEQ ID NO: 859 sets forth the nucleotide sequence of a C class oligonucleotide.
SEQ ID NO: 860 sets forth the nucleotide sequence of a C class oligonucleotide.
SEQ ID NO: 861 sets forth the nucleotide sequence of a P class oligonucleotide.

### Particular Embodiments of the Invention are Set Forth in the Following Numbered Paragraphs:

1. A immunogenic composition comprising a Clostridioides difficile (C. difficile) toxoid A and/or toxoid B, and a CpG adjuvant and/or a saponin containing liposomal adjuvant.
2. The immunogenic composition of paragraph 1, comprising a CpG adjuvant.
3. The immunogenic composition of paragraph 2, wherein the CpG adjuvant comprises at least one CpG.
4. The immunogenic composition of any one of paragraph 2 or 3, wherein the CpG adjuvant comprises a CpG oligonucleotide.
5. The immunogenic composition of any one of paragraphs 2-4, wherein the CpG adjuvant comprises a CpG oligonucleotide TLR9 agonist.
6. The immunogenic composition of any one of paragraphs 2-5, wherein the CpG adjuvant is a B class CpG.
7. The immunogenic composition of any one of paragraphs 2-6, wherein the CpG adjuvant is selected from SEQ ID NOS: 48 and 841-861.
8. The immunogenic composition of any one of paragraphs 2-7, wherein the CpG adjuvant comprises CpG 24555 (SEQ ID NO: 48), CpG 1018 (SEQ ID NO: 841), CpG 7909 (SEQ ID NO: 842), CpG 10103 (SEQ ID NO: 843), or CpG 1826 (SEQ ID NO: 844).
9. The immunogenic composition of paragraph 8, wherein the CpG adjuvant comprises CpG 24555.
10. The immunogenic composition of any one of paragraphs 2-9, comprising about 0.1 to 5 mg/mL or higher the CpG.
11. The immunogenic composition of any one of paragraphs 2-9, comprising about 0.1 to about 1.0 mg/mL the CpG.
12. The immunogenic composition of any one of paragraphs 2-9, comprising about 0.5 mg/mL the CpG.
13. The immunogenic composition of any one of paragraphs 2-9, comprising about 0.5 to about 1.5 mg/mL the CpG.
14. The immunogenic composition of any one of paragraphs 2-9, comprising about 1.0 mg/mL the CpG.
15. The immunogenic composition of any one of paragraphs 2-9, comprising about 0.8 to about 1.8 mg/mL the CpG.
16. The immunogenic composition of any one of paragraphs 2-9, comprising about 1.2 mg/mL the CpG.
17. The immunogenic composition of any one of paragraphs 2-9, comprising about 3.0 to about 4.0 mg/mL the CpG.
18. The immunogenic composition of any one of paragraphs 2-9, comprising about 3.6 mg/mL the CpG.
19. The immunogenic composition of any one of paragraphs 2-18, wherein the CpG adjuvant further comprises an additional adjuvant.
20. The immunogenic composition of any one of paragraphs 2-19, wherein the CpG adjuvant further comprises an aluminum salt.
21. The immunogenic composition of paragraph 20, wherein the aluminum salt is aluminum phosphate, aluminum sulfate or aluminum hydroxide.
22. The immunogenic composition of any one of paragraphs 1-21, wherein the CpG adjuvant further comprises aluminum hydroxide (Al(OH)₃).
23. The immunogenic composition of any one of paragraphs 2-22, comprising a CpG and about 0.1 to 5 mg/mL or higher of Al(OH)₃.
24. The immunogenic composition of any one of paragraphs 2-22, comprising a CpG and about 0.5 to about 1.5 mg/mL of Al(OH)₃.
25. The immunogenic composition of any one of paragraphs 2-22, comprising a CpG and about 1.0 of Al(OH)₃.
26. The immunogenic composition of any one of paragraphs 2-22, comprising a CpG and about 1.0 to about 2.5 mg/mL of Al(OH)₃.
27. The immunogenic composition of any one of paragraphs 2-22, comprising a CpG and about 1.5 to about 2.5 mg/mL of Al(OH)₃.
28. The immunogenic composition of any one of paragraphs 2-22, comprising a CpG and about 1.5 of Al(OH)₃.
29. The immunogenic composition of any one of paragraphs 2-22, comprising a CpG and about 1.7 of Al(OH)₃.
30. The immunogenic composition of any one of paragraphs 2-22, comprising a CpG and about 1.8 of Al(OH)₃.
31. The immunogenic composition of any one of paragraphs 2-32, comprising a CpG and about 2.0 of Al(OH)₃.
32. The immunogenic composition of any one of paragraphs 2-22, comprising a CpG and about 2.5 of Al(OH)₃.
33. The immunogenic composition of any one of paragraphs 2-22, comprising about 0.5 mg/mL CpG and about 0.1 to 5 mg/mL or higher of Al(OH)₃.
34. The immunogenic composition of any one of paragraphs 2-22, comprising about 1.0 mg/mL CpG and about 1.5 mg/mL of Al(OH)₃.
35. The immunogenic composition of any one of paragraphs 2-22, comprising about 1.2, 1.3, 1.4, 1.5, or 1.6 mg/mL CpG and about 1.7 mg/mL of Al(OH)₃.
36. The immunogenic composition of any one of paragraphs 2-22, comprising about 1.5, 1.6, 1.7, 1.8, or 1.9 mg/mL CpG and about 2.0 mg/mL of Al(OH)₃.
37. The immunogenic composition of any one of paragraphs 2-22, comprising about 1.8, 1.9, 2.0, 2.1, 2.2, or 2.4 mg/mL CpG and about 2.5 mg/mL of Al(OH)₃.
38. The immunogenic composition of any one of paragraphs 2-22, comprising about 3.6 mg/mL CpG and about 1.8 mg/mL of Al(OH)₃.
39. The immunogenic composition of any one of paragraphs 2-22, comprising about 1.4 mg/mL CpG and about 1.7 mg/mL of Al(OH)₃ or higher.
40. The immunogenic composition of any one of paragraphs 2-122, comprising about 1.6 mg/mL CpG and about 2.0 mg/mL of Al(OH)₃ or higher.
41. The immunogenic composition of any one of paragraphs 2-22, comprising about 2.0 mg/mL CpG and about 2.5 mg/mL of Al(OH)₃ or higher.
42. The immunogenic composition of any one of paragraphs 2-22, comprising a mass ratio of CpG/Al(OH)₃ of about 1.21 or higher.
43. The immunogenic composition of any one of paragraphs 2-42, wherein the CpG adjuvant comprises a histidine or phosphate buffer.
44. The immunogenic composition of paragraph 43, wherein the histidine buffer is at a concentration of about 1 mM to 100 mM.
45. The immunogenic composition of paragraph 43, wherein the histidine buffer is at a concentration of about 5 mM to 15 mM.
46. The immunogenic composition of paragraph 43, wherein the histidine buffer is at a concentration of about 10 mM.
47. The immunogenic composition of paragraph 43, wherein the phosphate buffer is at a concentration of about 1 mM to 100 mM.
48. The immunogenic composition of paragraph 43, wherein the phosphate buffer is at a concentration of about 5 mM to 15 mM.
49. The immunogenic composition of paragraph 43, wherein the phosphate buffer is at a concentration of about 10 mM.
50. The immunogenic composition of any one of paragraphs 2-43, wherein the CpG adjuvant comprises sodium chloride.
51. The immunogenic composition of paragraph 50, wherein the sodium chloride is at a concentration of about 10 mM to 300 mM.
52. The immunogenic composition of paragraph 51, wherein the sodium chloride is at a concentration of about 20 mM to 100 mM.
53. The immunogenic composition of paragraph 51, wherein the sodium chloride is at a concentration of about 50 mM.
54. The immunogenic composition of paragraph 51, wherein the sodium chloride is at a concentration of about 60 mM.
55. The immunogenic composition of any one of paragraphs 2-54, wherein the CpG adjuvant has a pH of about 6.0 to 7.0.
56. The immunogenic composition of paragraph 55, wherein the pH is about 6.5.
57. The immunogenic composition of paragraph 2, wherein the CpG adjuvant comprises about 0.5, 1.0, 1.2 or 3.6 mg/mL CpG 24555.
58. The immunogenic composition of paragraph 57, wherein the CpG adjuvant comprises 3.6 mg/mL CpG 24555.
59. The immunogenic composition of paragraph 57 or 58, wherein the CpG adjuvant comprises 3.6 mg/mL CpG 24555, a histidine buffer and sodium chloride.
60. The immunogenic composition of any one of paragraphs 57-59, wherein the CpG adjuvant comprises about 3.6 mg/mL CpG 24555, 10 mM histidine buffer, 60 mM sodium chloride at a pH of about 6.5.
61. The immunogenic composition of paragraph 2, wherein the CpG adjuvant comprises about 0.5 mg/mL CpG 24555 and about 1.0 mg/mL of Al(OH)₃, about 1.0 mg/mL CpG 24555 and about 1.0 mg/mL of Al(OH)₃, about 3.6 mg/mL CpG 24555 and about 1.0 mg/mL of Al(OH)₃ or about 1.0 mg/mL CpG 24555 and about 1.5 mg/mL of Al(OH)₃.
62. The immunogenic composition of paragraph 61, wherein the CpG adjuvant comprises about 1.0 mg/mL CpG 24555 and about 1.5 mg/mL of Al(OH)₃.
63. The immunogenic composition of paragraph 61 or 62, wherein the CpG adjuvant comprises about 1.0 mg/mL CpG 24555 and about 1.5 mg/mL of Al(OH)₃, a histidine buffer and sodium chloride.
64. The immunogenic composition of any one of paragraphs 61-63, wherein the CpG adjuvant comprises about 1.0 mg/mL CpG 24555 and about 1.5 mg/mL of Al(OH)₃, 10 mM histidine buffer, 50 mM sodium chloride at a pH of about 6.5.
65. The immunogenic composition of paragraph 1, comprising a saponin containing liposomal adjuvant.
66. The immunogenic composition of paragraph 65, wherein the saponin containing liposomal adjuvant comprises a saponin and a monophosphoryl lipid A (MPLA)-containing liposome composition.
67. The immunogenic composition of paragraph 65 or 66, wherein the saponin containing liposomal adjuvant comprises a saponin and a monophosphoryl lipid A (MPLA)-containing liposome composition, wherein the liposome composition comprises i) a lipid bilayer comprising phospholipids and ii) cholesterol.
68. The immunogenic composition of any one of paragraphs 65-67, wherein the saponin is selected from QS-7, QS-18, QS-21, or a mixture thereof.
69. The immunogenic composition of any one of paragraphs 65-68, wherein the saponin is QS-21.
70. The immunogenic composition of any one of paragraphs 67-69, wherein the phospholipid is selected from dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG).
71. The immunogenic composition of any one of paragraphs 67-70, wherein the phospholipids are DMPC and DMPG.
72. The immunogenic composition any one of paragraphs 65-71, wherein the saponin containing liposomal adjuvant comprises QS-21, Monophosphoryl Lipid A (MPLA), DMPC, DMPG and cholesterol.
73. The immunogenic composition of any one of paragraphs 66-72, wherein the MPLA is Monophosphoryl 3-Deacyl Lipid A.
74. The immunogenic composition of any one of paragraphs 65-73, comprising about 0.05 to about 1.0 mg/mL or higher of QS-21.
75. The immunogenic composition of any one of paragraphs 65-73, comprising about 0.1 to about 0.4 mg/mL of QS-21.
76. The immunogenic composition of any one of paragraphs 65-73, comprising about 0.2 mg/mL of QS-21.
77. The immunogenic composition of any one of paragraphs 65-76, comprising about 0.1 to about 1.0 mg/mL or higher of MPLA.
78. The immunogenic composition of any one of paragraphs 65-76, comprising about 0.2 to about 0.6 mg/mL of MPLA.
79. The immunogenic composition of any one of paragraphs 65-76, comprising about 0.3 to about 0.5 mg/mL of MPLA.
80. The immunogenic composition of any one of paragraphs 65-76, comprising about 0.4 mg/mL of MPLA.
81. The immunogenic composition of any one of paragraphs 65-80, comprising about 0.5 to about 20 mg/mL or higher of cholesterol.
82. The immunogenic composition of any one of paragraphs 65-80, comprising about 5 to about 15 mg/mL of cholesterol.
83. The immunogenic composition of any one of paragraphs 65-80, comprising about 11 mg/mL of cholesterol.
84. The immunogenic composition of any one of paragraphs 65-83, comprising about 0.5 to about 20 mg/mL or higher of DMPC.
85. The immunogenic composition of any one of paragraphs 65-83, comprising about 5 to about 15 mg/mL of DMPC.
86. The immunogenic composition of any one of paragraphs 65-83, comprising about 14 mg/mL of DMPC.
87. The immunogenic composition of any one of paragraphs 65-86, comprising about 0.5 to about 3.0 mg/mL or higher of DMPG.
88. The immunogenic composition of any one of paragraphs 65-86, comprising about 1.0 to about 2.0 mg/mL of DMPG.
89. The immunogenic composition of any one of paragraphs 65-86 comprising about 1.6 mg/mL of DMPG.
90. The immunogenic composition of any one of paragraphs 65 to 89, wherein the saponin containing liposomal adjuvant comprises a histidine or phosphate buffer.
91. The immunogenic composition of paragraph 90, wherein the phosphate buffer is at a concentration of about 1 mM to 100 mM.
92. The immunogenic composition of paragraph 90, wherein the phosphate buffer is at a concentration of about 5 mM to 15 mM.
93. The immunogenic composition of paragraph 90, wherein the phosphate buffer is at a concentration of about 10 mM.
94. The immunogenic composition of any one of paragraphs 65 to 93, wherein the saponin containing liposomal adjuvant comprises sodium chloride.
95. The immunogenic composition of paragraph 94, wherein the sodium chloride is at a concentration of about 10 mM to 300 mM.
96. The immunogenic composition of paragraph 51, wherein the sodium chloride is at a concentration of about 100 mM to 200 mM.
97. The immunogenic composition of paragraph 51, wherein the sodium chloride is at a concentration of about 150 mM.
98. The immunogenic composition of any one of paragraphs 65-97, wherein the saponin containing liposomal adjuvant has a pH of about 5.5 to 7.0.
99. The immunogenic composition of paragraph 55, wherein the pH is about 6.2.
100. The immunogenic composition of paragraph 65, wherein the saponin containing liposomal adjuvant comprises about 0.2 mg/mL QS-21, about 0.4 mg/mL Monophosphoryl 3-Deacyl Lipid A, about 14 mg/mL DMPC, about 1.6 mg/mL DMPG and about 11 mg/mL cholesterol.
101. The immunogenic composition of paragraph 65, wherein the saponin containing liposomal adjuvant comprises about 0.2 mg/mL QS-21, about 0.4 mg/mL Monophosphoryl 3-Deacyl Lipid A, about 14 mg/mL DMPC, about 1.6 mg/mL DMPG, about 11 mg/mL cholesterol, 10 mM phosphate and 150 mM sodium chloride.
102. The immunogenic composition of paragraph 65, wherein the saponin containing liposomal adjuvant comprises about 0.2 mg/mL QS-21, about 0.4 mg/mL Monophosphoryl 3-Deacyl Lipid A, about 14 mg/mL DMPC, about 1.6 mg/mL DMPG, about 11 mg/mL cholesterol, 10 mM phosphate and 150 mM sodium chloride at a pH of about 6.2.
103. The immunogenic composition of paragraph 65, wherein the saponin containing liposomal adjuvant comprises about 0.4 mg/mL QS-21, about 0.8 mg/mL Monophosphoryl 3-Deacyl Lipid A, about 28 mg/mL DMPC, about 3.2 mg/mL DMPG and about 22 mg/mL cholesterol.
104. The immunogenic composition of paragraph 65, wherein the saponin containing liposomal adjuvant comprises about 0.1 mg/mL QS-21, about 0.2 mg/mL Monophosphoryl 3-Deacyl Lipid A, about 7 mg/mL DMPC, about 0.8 mg/mL DMPG and about 5.5 mg/mL cholesterol.
105. The immunogenic composition of any one of paragraphs 1-104, wherein the C. difficile toxoid A comprises the amino acid sequence of SEQ ID NO: 4, wherein the initial methionine is absent, and C. difficile toxoid B comprises the amino acid sequence of SEQ ID NO: 6, wherein the initial methionine is absent.
106. The immunogenic composition of any one of paragraphs 1-105, wherein the C. difficile toxoid A comprises the amino acid sequence of SEQ ID NO: 84, and C. difficile toxoid B comprises the amino acid sequence of SEQ ID NO: 86.
107. The immunogenic composition of any one of paragraphs 1 to 106, wherein the composition comprises the C. difficile toxoid A and the C. difficile toxoid B in a ratio of about 3:1 to about 1:1.
108. The immunogenic composition of any one of paragraphs 1 to 107, wherein the composition comprises 200 μg of toxoid per dose.
109. The immunogenic composition of any one of paragraphs 1 to108, further comprising at least one of a buffer, a stabilizer, and a surfactant.
110. The immunogenic composition of any one of paragraphs 1 to 73, further comprising 10 mM Tris, 4.5% (/v) trehalose dihydrate and polysorbate 80.
111. The immunogenic composition of any one of paragraphs 1 to 110, wherein the C. difficile toxoid A and toxoid B are lyophilized and reconstituted with a CpG adjuvant or a saponin containing liposomal adjuvant.
112. The immunogenic composition of paragraph 111, wherein the lyophilized C. difficile toxoid A and toxoid B is reconstituted with a CpG adjuvant comprising about 3.6 mg/mL of CpG 24555.
113. The immunogenic composition of paragraph 112, wherein the lyophilized C. difficile toxoid A and toxoid B comprising 0.4 mg/mL total (200 μg/mL of toxoid A and 200 μg/mL of toxoid B) is reconstituted with a CpG adjuvant comprising about 3.6 mg/mL of CpG 24555.
114. The immunogenic composition of any one of paragraphs 111, wherein the lyophilized C. difficile toxoid A and toxoid B is reconstituted with a CpG adjuvant comprising about 1.0 mg/mL of CpG 24555 and about 1.5 mg/mL of Al(OH)₃.
115. The immunogenic composition of paragraph 114, wherein the lyophilized C. difficile toxoid A and toxoid B comprising 0.4 mg/mL total (200 μg/mL of toxoid A and 200 μg/mL of toxoid B) is reconstituted with a CpG adjuvant comprising about 1.0 mg/mL of CpG 24555 and about 1.5 mg/mL of Al(OH)₃.
116. The immunogenic composition of paragraph 111, wherein the lyophilized C. difficile toxoid A and toxoid B is reconstituted with a saponin containing liposomal adjuvant comprising about 0.2 mg/mL QS-21, about 0.4 mg/mL Monophosphoryl 3-Deacyl Lipid A, about 14 mg/mL DMPC, about 1.6 mg/mL DMPG and about 11 mg/mL cholesterol.
117. The immunogenic composition of paragraph 116, wherein the lyophilized C. difficile toxoid A and toxoid B comprising 0.4 mg/mL total (200 μg/mL of toxoid A and 200 μg/mL of toxoid B) is reconstituted with a saponin containing liposomal adjuvant comprising about 0.2 mg/mL QS-21, about 0.4 mg/mL Monophosphoryl 3-Deacyl Lipid A, about 14 mg/mL DMPC, about 1.6 mg/mL DMPG and about 11 mg/mL cholesterol.
118. The immunogenic composition of any one of paragraphs 1-117, wherein the composition is administered at a dose volume of 0.5 mL.
119. The immunogenic composition of any one of paragraphs 1-118, for use as a medicament.
120. The immunogenic composition of any one of paragraphs 1-118, for use in a method of eliciting an immune response in a human subject against C. difficile in a human.
121. The immunogenic composition of any one of paragraphs 1 to 118, for use as a vaccine.
122. The immunogenic composition of any one of paragraphs 119-121, wherein the human subject is administered in a first and second dose of the immunogenic composition.
123. The immunogenic composition of any one of paragraphs 119-121, wherein the immunogenic composition is administered in a first and second dose.
124. The immunogenic composition of paragraph 123, wherein the second dose is administered about 2 months after the first dose (e.g. M 0, 2).
125. The immunogenic composition of paragraphs 123, wherein the second dose is administered about 6 months after the first dose (M 0, 6).
126. The immunogenic composition of any one of paragraphs 119-121, wherein the human subject is 18 years of age or older 50 years of age or older, 55 years of age or older, 60 years of age or older, 65 years of age or older, or 70 years of age or older.
127. A method of eliciting an immune response in a human subject against C. difficile, the method comprising administering to the human subject an immunogenic composition of any one of paragraphs 1-118.
128. A method of preventing, treating or ameliorating an infection, disease or condition associated with C. difficile in a human subject, the method comprising administering to the human subject an immunogenic composition of any one of paragraphs 1-118.
129. A method of preventing, treating or ameliorating a medically attended C. difficile infection in a human subject, the method comprising administering to the human subject an immunogenic composition of any one of paragraphs 1-118.
130. The method of any one of paragraphs 127-129, wherein the immunogenic composition is administered in a first and second dose.
131. The method of paragraph 130, wherein the second dose is administered about 2 months after the first dose (e.g. M 0, 2).
132. The method of paragraphs 130, wherein the second dose is administered about 6 months after the first dose (M 0, 6).
133. The method of any one of paragraphs 127-132, wherein the human subject is 18 years of age or older 50 years of age or older, 55 years of age or older, 60 years of age or older, 65 years of age or older, or 70 years of age or older.
134. The method of any one of paragraphs 127-133, wherein an immune response is elicited and comprises neutralizing antibodies against C. difficile toxin A and/or neutralizing antibodies against C. difficile toxin B.
135. The method of any one of paragraphs 127-134, wherein neutralizing antibodies elicited against C. difficile toxin A and/or B is greater after administration of 2 doses of the immunogenic composition compared to administration of 3 doses of the investigational C. difficile vaccine.

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every whole number integer within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of” and “consists of”, respectively, in every instance.

All publications and patent applications mentioned in the specification are indicative of the level of those skilled in the art to which this invention pertains. All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLES

### Example 1—Immunogenicity of C. difficile Vaccine Antigens Formulated with a Saponin Containing Liposomal Adjuvant

The relative immunogenicity of C. difficile toxoid antigens formulated with a saponin containing liposomal adjuvant (LiNA-2), as described herein, was compared to formulations with aluminum hydroxide (Al(OH)₃) in nonhuman primates (NHPs). The toxin neutralization assay (TNA) described herein was used to analyze sera collected to determine functional antitoxin activity. The TNA demonstrates the ability of an antiserum to neutralize the cytotoxic effect mediated by wild type C. difficile toxin A (TcdA) or toxin B (TcdB) in vitro. The TNA provides a quantitative assessment of the functional activity of antibodies that are present in a serum sample. The geometric mean concentrations (GMTs) with 95% confidence intervals (CI) are provided for each C. difficile toxoid antigen.

Rhesus macaques were immunized IM with Toxoid A (Txd A) and Toxoid B (Txd B) according to the study design in Table 1. Group 1 received C. difficile vaccine antigens formulated with Al(OH)₃. Groups 2, 3 and 4 received the C. difficile vaccine antigens formulated with LiNA-2 adjuvant at Month 0, 1, 6, Month 0, 2 and Month 0, 6, respectively.

TABLE 1
NHP study design with LiNA-2 adjuvanted formulations

| | Number NHPs | | | Dose | Dosing |
|---|---|---|---|---|---|
| Group | per group | Formulation | Composition (per dose) | Route | Schedule |
| 1 | 5 | Al(OH)₃ | 10 μg of each Txd A&B | 0.5 mL | Month |
| | | | 0.5 mg Al(OH)₃ | IM | 0, 1, 6 |
| 2 | 5 | LiNA-2 | 10 μg of each Txd A&B | 0.5 mL | Month |
| | | | 0.2 mg (200 μg) 3D-PHAD ® | IM | 0, 1, 6 |
| | | | 0.1 mg (100 μg) QS-21 | | |
| | | | 7 mg DMPC | | |
| | | | 0.89 mg DMPG | | |
| | | | 5.41 mg Cholesterol | | |
| 3 | 5 | LiNA-2 | 10 μg of each Txd A&B | 0.5 mL | Month |
| | | | 0.2 mg (200 μg) 3D-PHAD ® | IM | 0, 2 |
| | | | 0.1 mg (100 μg) QS-21 | | |
| | | | 7 mg DMPC | | |
| | | | 0.89 mg DMPG | | |
| | | | 5.41 mg Cholesterol | | |
| 4 | 5 | LiNA-2 | 10 μg of each Txd A&B | 0.5 mL | Month |
| | | | 0.2 mg (200 μg) 3D-PHAD ® | IM | 0, 6 |
| | | | 0.1 mg (100 μg) QS-21 | | |
| | | | 7 mg DMPC | | |
| | | | 0.89 mg DMPG | | |
| | | | 5.41 mg Cholesterol | | |

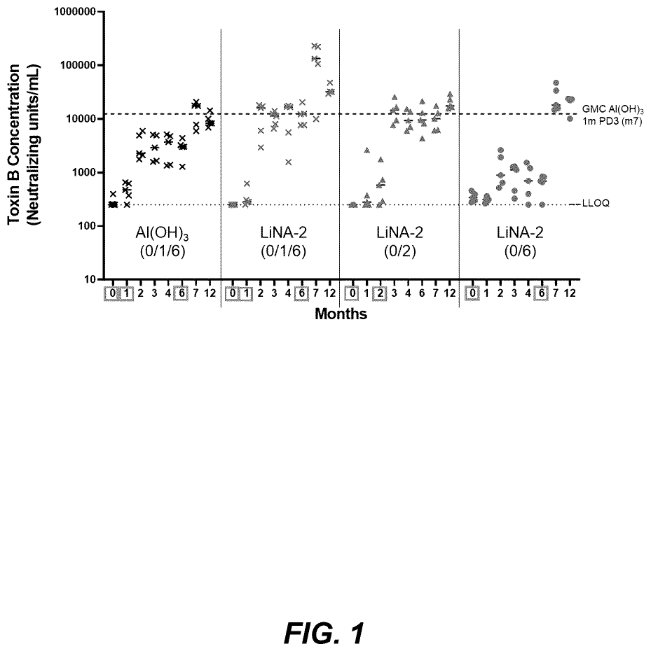

Sera were collected at multiple time points and the ability to neutralize toxin cytotoxic activity was measured in TNAs. The neutralization titers of individual animals for Toxin B are shown in FIG. 1 and GMTs are provided in Table 2, and illustrate that toxoids formulated with LUNA-2 elicit a robust and more rapid immune response able to neutralize Toxin B cytotoxicity after 2 doses compared to 3 doses formulated with Al(OH)₃. GMTs for Toxin A are provided in Table 3.

TABLE 2
Toxin B neutralizing titers for LiNA-2 adjuvanted formulations

| | Toxin B concentration (Neutralizing units/mL) | | | | | | | |
|---|---|---|---|---|---|---|---|---|
| | Al(OH)₃ | | LiNA-2 | | LiNA-2 | | LiNA-2 | |
| | 0/1/6 | | 0/1/6 | | 0/2 | | 0/6 | |
| Months | GMT | 95% CI | GMT | 95% CI | GMT | 95% CI | GMT | 95% CI |
| 0 | 276 | 214- | 250 | 250- | 250 | 250- | 347 | 270- |
| | | 356 | | 250 | | 250 | | 445 |
| 1 | 447 | 273- | 327 | 208- | 445 | 127- | 308 | 267- |
| | | 731 | | 513 | | 1560 | | 354 |
| 2 | 3016 | 1528- | 9729 | 3551- | 561 | 212- | 1081 | 454- |
| | | 5950 | | 26659 | | 1484 | | 2573 |
| 3 | 2847 | 1414- | 10052 | 6784- | 13498 | 7467- | 771 | 344- |
| | | 5735 | | 14895 | | 24401 | | 1728 |
| 4 | 2793 | 1229- | 8441 | 2263- | 9633 | 5811- | 661 | 259- |
| | | 6351 | | 31484 | | 15972 | | 1682 |
| 6 | 2774 | 1569- | 11279 | 6774- | 9878 | 4762- | 601 | 322- |
| | | 4905 | | 18780 | | 20491 | | 1120 |
| 7 | 12421 | 6179- | 93736 | 18718- | 9701 | 5473- | 23186 | 12215- |
| | | 24969 | | 469416 | | 17196 | | 44009 |
| 12 | 9253 | 6552- | 29754 | 18434- | 19904 | 14325- | 17400 | 10587- |
| | | 13067 | | 48028 | | 27657 | | 28596 |

TABLE 3
Toxin A neutralizing titers for LiNA-2 adjuvanted formulations

| | Toxin A concentration (Neutralizing units/mL) | | | | | | | |
|---|---|---|---|---|---|---|---|---|
| | Al(OH)₃ | | LiNA-2 | | LiNA-2 | | LiNA-2 | |
| | 0/1/6 | | 0/1/6 | | 0/2 | | 0/6 | |
| Months | GMT | 95% CI | GMT | 95% CI | GMT | 95% CI | GMT | 95% CI |
| 0 | 136 | 96- | 117 | 78- | 143 | 118- | 177 | 119- |
| | | 191 | | 177 | | 172 | | 263 |
| 1 | 228 | 109- | 733 | 291- | 883 | 673- | 796 | 316- |
| | | 477 | | 1848 | | 1158 | | 2008 |
| 2 | 2099 | 1135- | 4468 | 2518- | 705 | 300- | 962 | 567- |
| | | 3883 | | 7926 | | 1653 | | 1633 |
| 3 | 927 | 393- | 2442 | 1451- | 3389 | 1188- | 979 | 456- |
| | | 2185 | | 4110 | | 9667 | | 2102 |
| 4 | 503 | 177-1 | 1834 | 1158- | 1954 | 1166- | 703 | 385- |
| | | 430 | | 2905 | | 3272 | | 1286 |
| 6 | 461 | 174- | 1742 | 739- | 1814 | 868- | 581 | 356- |
| | | 1218 | | 4108 | | 3789 | | 948 |
| 7 | 2348 | 1486- | 6032 | 2242- | 1336 | 525- | 18895 | 8332- |
| | | 3710 | | 16230 | | 3401 | | 42851 |
| 12 | 1097 | 568- | 4452 | 3215- | 2644 | 1030- | 4125 | 2933- |
| | | 2120 | | 6167 | | 6784 | | 5801 |

### Example 2—Immunogenicity of C. difficile Vaccine Antigens Formulated with a Saponin Containing Liposomal Adjuvant or CpG Adjuvant+Al(OH)₃ Administered at Month 0, 2 and Month 0, 6

The relative immunogenicity of C. difficile toxoid antigens formulated with a saponin containing liposomal adjuvant (LiNA-2) or CpG adjuvant (CpG 24555) combined with Al(OH)₃ was compared to formulations with Al(OH)₃ in NHPs. The toxin neutralization assay described in Example 1 was used to measure the functional cytotoxic activity of sera at multiple time points following immunization. The geometric mean concentrations (GMTs) with 95% confidence intervals (CI) are provided for each C. difficile toxoid antigen.

Rhesus macaques were immunized IM with Txd A and Txd B according to the study design in Table 4. Group 1 received C. difficile vaccine antigens formulated with Al(OH)₃, at Month 0, 1, 6. Groups 2 and 3 received the C. difficile vaccine antigens formulated with LiNA-2 adjuvant at Month 0, 2 and Month 0, 6, respectively. Groups 4 and 5 received the C. difficile vaccine antigens formulated with 1 mg/mL CpG adjuvant+1 mg/mL Al(OH)₃ at Month 0, 2 and Month 0, 6, respectively.

TABLE 4
NHP study design with LiNA-2 and CpG/Al(OH)₃ adjuvanted formulations

| | Number NHPs | | | Dose | Dosing |
|---|---|---|---|---|---|
| Group | per group | Formulation | Composition (per dose) | Route | Schedule |
| 1 | 5 | Al(OH)₃ | 10 μg of each Txd A&B | 0.5 mL | Month |
| | | | 0.5 mg Al(OH)₃ | IM | 0, 1, 6 |
| 2 | 5 | LiNA-2 | 10 μg of each Txd A&B | 0.5 mL | Month |
| | | | 0.2 mg (200 μg) 3D-PHAD ® | IM | 0, 2 |
| | | | 0.1 mg (100 μg) QS-21 | | |
| | | | 7 mg DMPC | | |
| | | | 0.89 mg DMPG | | |
| | | | 5.41 mg Cholesterol | | |
| 3 | 5 | LiNA-2 | 10 μg of each Txd A&B | 0.5 mL | Month |
| | | | 0.2 mg (200 μg) 3D-PHAD ® | IM | 0, 6 |
| | | | 0.1 mg (100 μg) QS-21 | | |
| | | | 7 mg DMPC | | |
| | | | 0.89 mg DMPG | | |
| | | | 5.41 mg Cholesterol | | |
| 4 | 5 | CpG/Al(OH)₃ | 10 μg of each Txd A&B | 0.5 mL | Month |
| | | | 0.5 mg CpG | IM | 0, 2 |
| | | | 0.5 mg Al(OH)₃ | | |
| 5 | 5 | CpG/Al(OH)₃ | 10 μg of each Txd A&B | 0.5 mL | Month |
| | | | 0.5 mg CpG | IM | 0, 6 |
| | | | 0.5 mg Al(OH)₃ | | |

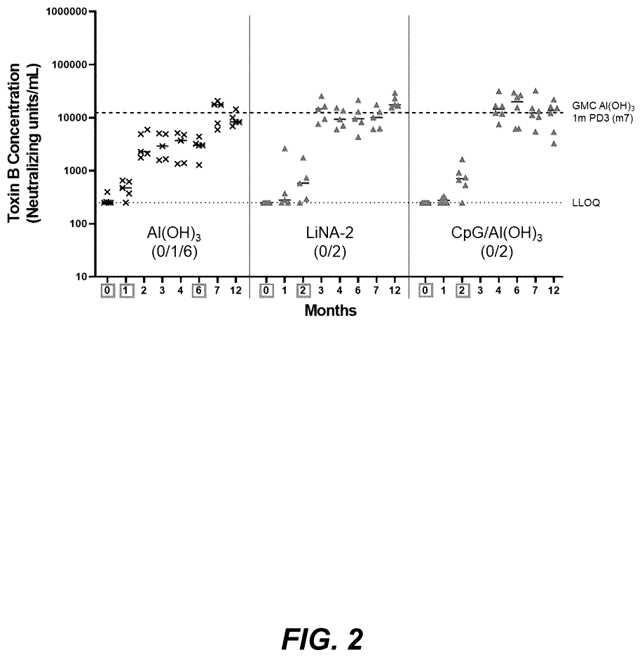

Sera were collected at multiple time points and the ability to neutralize toxin cytotoxic activity was measured in TNAs. The neutralization titers of individual animals for Toxin B immunized at Month 0, 2 are show in FIG. 2 and GMTs are provided in Table 5. Toxoid antigens administered with either the LiNA-2 or the CpG+Al(OH)₃ formulations on the Month 0, 2 schedule elicited a robust and more rapid immune response able to neutralize Toxin B cytotoxicity after 2 doses compared to 3 doses formulated with Al(OH)₃. Both the LiNA-2 and the CpG+Al(OH)₃ adjuvanted formulations elicited responses that are comparable to the responses determined following 3 doses of the Al(OH)₃ formulated toxoid antigens. The functional immune response measured in both of the adjuvanted formulation groups vaccinated using the abbreviated Month 0, 2 schedule were durable to study Month 12 (10 months following the second dose). GMTs for Toxin A are provided in Table 6.

TABLE 5
Toxin B neutralizing titers for LiNA-2 and
CpG/Al(OH)₃ adjuvanted formulations (0.2M)

| | Toxin B concentration (Neutralizing units/mL) | | | | | |
|---|---|---|---|---|---|---|
| | Al(OH)₃ | | LiNA-2 | | CpG/Al(OH)₃ | |
| | 0/1/6 | | 0/2 | | 0/2 | |
| Months | GMT | 95% CI | GMT | 95% CI | GMT | 95% CI |
| 0 | 276 | 214- | 250 | 250- | 250 | 250- |
| | | 356 | | 250 | | 250 |
| 1 | 447 | 273- | 445 | 127- | 279 | 244- |
| | | 731 | | 1560 | | 318 |
| 2 | 3016 | 1528- | 561 | 212- | 682 | 354- |
| | | 5950 | | 1484 | | 1313 |
| 3 | 2847 | 1414- | 13498 | 7467- | Time point not measured | |
| | | 5735 | | 24401 | | |
| 4 | 2793 | 1229- | 9633 | 5811- | 14586 | 8791- |
| | | 6351 | | 15972 | | 24201 |
| 6 | 2774 | 1569- | 9878 | 4762- | 15058 | 7104- |
| | | 4905 | | 20491 | | 31916 |
| 7 | 12421 | 6179- | 9701 | 5473- | 12666 | 6889- |
| | | 24969 | | 17196 | | 23290 |
| 12 | 9253 | 6552- | 19904 | 14325- | 10240 | 4749- |
| | | 13067 | | 27657 | | 22081 |

TABLE 6
Toxin A neutralizing titers for LiNA-2 and
CpG/Al(OH)₃ adjuvanted formulations (0.2M)

| | Toxin A concentration (Neutralizing units/mL) | | | | | |
|---|---|---|---|---|---|---|
| | Al(OH)₃ | | LiNA-2 | | CpG/Al(OH)₃ | |
| | 0/1/6 | | 0/2 | | 0/2 | |
| Months | GMT | 95% CI | GMT | 95% CI | GMT | 95% CI |
| 0 | 136 | 96- | 143 | 118- | 307 | 211- |
| | | 191 | | 172 | | 447 |
| 1 | 228 | 109- | 883 | 673- | 453 | 293- |
| | | 477 | | 1158 | | 701 |
| 2 | 2099 | 1135- | 705 | 300- | 695 | 580- |
| | | 3883 | | 1653 | | 833 |
| 3 | 927 | 393- | 3389 | 1188- | Time point not measured | |
| | | 2185 | | 9667 | | |
| 4 | 503 | 177- | 1954 | 1166- | 1896 | 1544- |
| | | 1430 | | 3272 | | 2328 |
| 6 | 461 | 174- | 1814 | 868- | 1136 | 762- |
| | | 1218 | | 3789 | | 1693 |
| 7 | 2348 | 1486- | 1336 | 525- | 891 | 566- |
| | | 3710 | | 3401 | | 1405 |
| 12 | 1097 | 568- | 2644 | 1030- | 467 | 261- |
| | | 2120 | | 6784 | | 836 |

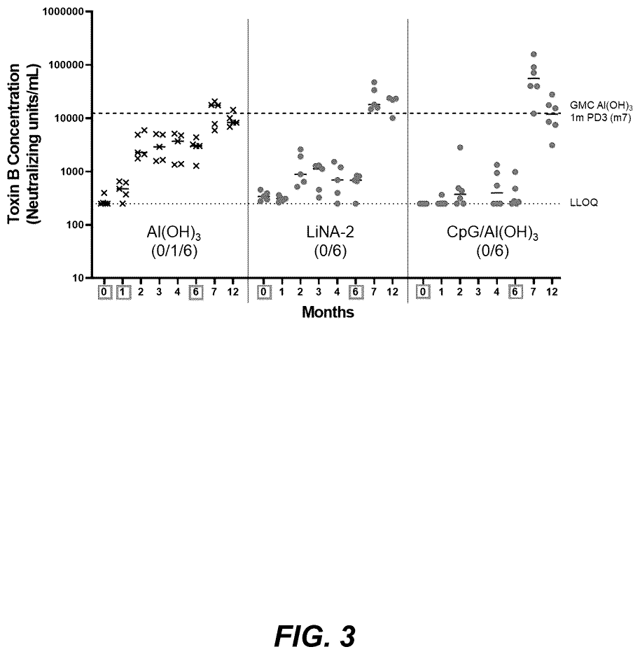

The neutralization titers of individual animals for Toxin B immunized at Month 0, 6 are show in FIG. 3 and GMTs are provided in Table 7. Toxoid antigens administered with either LiNA-2 or the CpG+Al(OH)₃ formulations on the Month 0, 6 schedule also elicited a robust and rapid functional immune response after 2 doses compared to 3 doses of the Al(OH)₃ formulated toxoid antigens. GMTs for Toxin A are provided in Table 8.

TABLE 7
Toxin B neutralizing titers for LiNA-2 and
CpG/Al(OH)₃ adjuvanted formulations (0.6M)

| | Toxin B concentration (Neutralizing units/mL) | | | | | |
|---|---|---|---|---|---|---|
| | Al(OH)₃ | | LiNA-2 | | CpG/Al(OH)₃ | |
| | 0/1/6 | | 0/6 | | 0/6 | |
| Months | GMT | 95% CI | GMT | 95% CI | GMT | 95% CI |
| 0 | 276 | 214- | 347 | 270- | 250 | 250- |
| | | 356 | | 445 | | 250 |
| 1 | 447 | 273- | 308 | 267- | 266 | 226- |
| | | 731 | | 354 | | 314 |
| 2 | 3016 | 1528- | 1081 | 454- | 477 | 182- |
| | | 5950 | | 2573 | | 1247 |
| 3 | 2847 | 1414- | 771 | 344- | Time point not measured | |
| | | 5735 | | 1728 | | |
| 4 | 2793 | 1229- | 661 | 259- | 469 | 214- |
| | | 6351 | | 1682 | | 1027 |
| 6 | 2774 | 1569- | 601 | 322- | 361 | 203- |
| | | 4905 | | 1120 | | 642 |
| 7 | 12421 | 6179- | 23186 | 12215- | 52074 | 20631- |
| | | 24969 | | 44009 | | 131439 |
| 12 | 9253 | 6552- | 17400 | 10587- | 10709 | 4756- |
| | | 13067 | | 28596 | | 24116 |

TABLE 8
Toxin A neutralizing titers for LiNA-2 and
CpG/Al(OH)₃ adjuvanted formulations (0.6M)

| | Toxin A concentration (Neutralizing units/mL) | | | | | |
|---|---|---|---|---|---|---|
| | Al(OH)₃ | | LiNA-2 | | CpG/Al(OH)₃ | |
| | 0/1/6 | | 0/6 | | 0/6 | |
| Months | GMT | 95% CI | GMT | 95% CI | GMT | 95% CI |
| 0 | 136 | 96- | 177 | 119- | 319 | 217- |
| | | 191 | | 263 | | 469 |
| 1 | 228 | 109- | 796 | 316- | 593 | 408- |
| | | 477 | | 2008 | | 864 |
| 2 | 2099 | 1135- | 962 | 567- | 726 | 545- |
| | | 3883 | | 1633 | | 968 |
| 3 | 927 | 393- | 979 | 456- | Time point not measured | |
| | | 2185 | | 2102 | | |
| 4 | 503 | 177- | 703 | 385- | 420 | 313- |
| | | 1430 | | 1286 | | 563 |
| 6 | 461 | 174- | 581 | 356- | 321 | 225- |
| | | 1218 | | 948 | | 457 |
| 7 | 2348 | 1486- | 18895 | 8332- | 5959 | 1709- |
| | | 3710 | | 42851 | | 20775 |
| 12 | 1097 | 568- | 4125 | 2933- | 1421 | 877- |
| | | 2120 | | 5801 | | 2303 |

### Example 3—Immunogenicity of C. difficile Vaccine Antigens Formulated with Different Dosage Strengths of CpG Adjuvant+Al(OH)₃ Administered at Month 0, 6

The relative immunogenicity of C. difficile toxoid antigens formulated with two levels of CpG adjuvant (CpG 24555) combined with Al(OH)₃ were compared to formulations with Al(OH)₃ in NHPs. The toxin neutralization assay described in Example 1 was used to measure the functional cytotoxic activity of sera at multiple time points following immunization. The geometric mean concentrations (GMTs) with 95% confidence intervals (CI) are provided for each C. difficile toxoid antigen.

Rhesus macaques were immunized IM with Txd A and Txd B according to the study design in Table 9. Group 1 received 3 doses (Month 0, 1, 6) of the C. difficile vaccine antigens formulated with Al(OH)₃. Groups 2 and 3 received two doses (Month 0, 6) of the C. difficile vaccine antigens formulated with either 0.5 mg/mL CpG+1 mg/mL Al(OH)₃ or 1 mg/mL CpG+1 mg/mL Al(OH)₃, respectively.

TABLE 9
NHP study design with CpG/Al(OH)₃ adjuvanted formulations

| | Number NHPs | | | Dose | Dosing |
|---|---|---|---|---|---|
| Group | per group | Formulation | Composition (per dose) | Route | Schedule |
| 1 | 5 | Al(OH)₃ | 10 μg of each Txd A&B | 0.5 mL | Month |
| | | | 0.5 mg Al(OH)₃ | IM | 0, 1, 6 |
| 2 | 5 | CpG/Al(OH)₃ | 10 μg of each Txd A&B | 0.5 mL | Month |
| | | | 0.25 mg CpG | IM | 0, 6 |
| | | | 0.5 mg Al(OH)₃ | | |
| 3 | 5 | CpG/Al(OH)₃ | 10 μg of each Txd A&B | 0.5 mL | Month |
| | | | 0.5 mg CpG | IM | 0, 6 |
| | | | 0.5 mg Al(OH)3 | | |

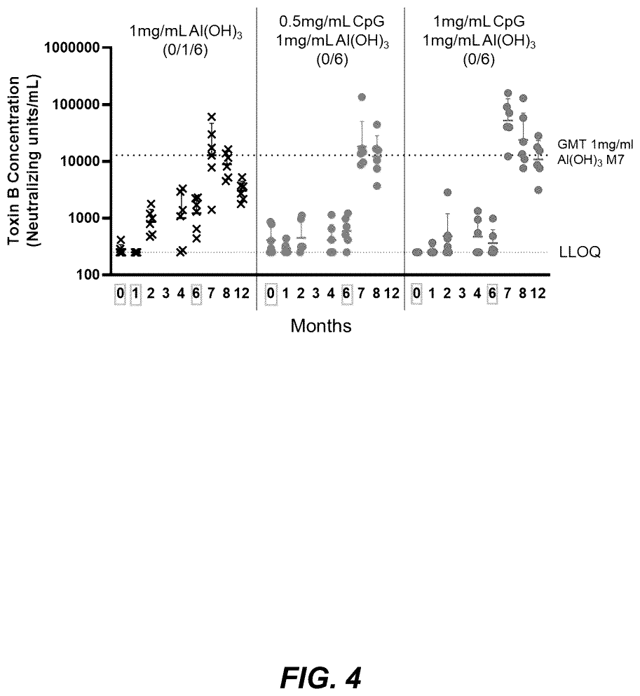

Sera were collected at multiple time points and the ability to neutralize toxin cytotoxic activity was measured in TNAs. The neutralization titers of individual animals for Toxin B are shown in FIG. 4 and GMTs are provided in Table 10, and illustrate that both of the CpG+Al(OH)₃ formulations elicited robust and rapid immune responses able to neutralize Toxin B cytotoxicity after just 2 doses. GMTs for Toxin A are provided in Table 11.

TABLE 10
Toxin B neutralizing titers for CpG/Al(OH)₃ adjuvanted formulations

| | Toxin B concentration (Neutralizing units/mL) | | | | | |
|---|---|---|---|---|---|---|
| | | | 0.5 mg/mL CpG | | 1 mg/mL CpG | |
| | Al(OH)₃ | | 1 mg/mL Al(OH)3 | | 1 mg/mL Al(OH)₃ | |
| | 0/1/6 | | 0/6 | | 0/6 | |
| Months | GMT | 95% CI | GMT | 95% CI | GMT | 95% CI |
| 0 | 278 | 225- | 411 | 229- | 250 | 250- |
| | | 343 | | 738 | | 250 |
| 1 | 250 | 249- | 292 | 232- | 266 | 226- |
| | | 251 | | 369 | | 314 |
| 2 | 858 | 503- | 447 | 224- | 477 | 182- |
| | | 1463 | | 890 | | 1247 |
| 3 | Time point not measured | | Time point not measured | | Time point not measured | |
| 4 | 993 | 306- | 409 | 211- | 469 | 214- |
| | | 3228 | | 793 | | 1027 |
| 6 | 1213 | 592- | 591 | 321- | 361 | 203- |
| | | 2485 | | 1090 | | 642 |
| 7 | 12770 | 3284- | 18184 | 6265- | 52074 | 20631- |
| | | 49655 | | 52777 | | 131439 |
| 8 | 8855 | 5087- | 12154 | 5024- | 23807 | 7582- |
| | | 15416 | | 29404 | | 74750 |
| 12 | 2998 | 1977- | 7942 | 4577- | 10709 | 4756- |
| | | 4545 | | 13780 | | 24116 |

TABLE 11
Toxin A neutralizing titers for CpG/Al(OH)₃ adjuvanted formulations

| | Toxin A concentration (Neutralizing units/mL) | | | | | |
|---|---|---|---|---|---|---|
| | | | 0.5 mg/mL CpG | | 1 mg/mL CpG | |
| | Al(OH)₃ | | 1 mg/mL Al(OH)3 | | 1 mg/mL Al(OH)₃ | |
| | 0/1/6 | | 0/6 | | 0/6 | |
| Months | GMT | 95% CI | GMT | 95% CI | GMT | 95% CI |
| 0 | 416 | 391- | 334 | 214- | 319 | 217- |
| | | 443 | | 524 | | 469 |
| 1 | 217 | 134- | 462 | 310- | 593 | 408- |
| | | 349 | | 689 | | 864 |
| 2 | 706 | 550- | 512 | 428- | 726 | 545- |
| | | 905 | | 613 | | 968 |
| 3 | Time point not measured | | Time point not measured | | Time point not measured | |
| 4 | 353 | 227- | 236 | 176- | 420 | 313- |
| | | 548 | | 318 | | 563 |
| 6 | 297 | 211- | 316 | 208- | 321 | 225- |
| | | 419 | | 481 | | 457 |
| 7 | 1233 | 530- | 4226 | 1392- | 5959 | 1709- |
| | | 2870 | | 12830 | | 20775 |
| 8 | 1417 | 584- | 3874 | 1902- | 3623 | 1674- |
| | | 3438 | | 7891 | | 7841 |
| 12 | 355 | 249- | 1720 | 898- | 1421 | 877- |
| | | 506 | | 3296 | | 2303 |

### Example 4—Immunogenicity of C. difficile Vaccine Antigens Formulated with Different Dosage Strengths of CpG Adjuvant Alone or CpG Adjuvant+Al(OH)₃ Administered at Month 0, 2

The relative immunogenicity of C. difficile toxoid antigens formulated with CpG (CpG 24555) alone or CpG 24555 combined with Al(OH)₃ at different levels of the CpG adjuvant were compared to formulations with Al(OH)₃ in NHPs. The toxin neutralization assay described in Example 1 was used to measure the functional cytotoxic activity of sera at multiple time points following immunization. The geometric mean concentrations (GMTs) with 95% confidence intervals (CI) are provided for each C. difficile toxoid antigen.

Rhesus macaques were immunized IM with Txd A and Txd B according to the study design in Table 12. Group 1 received C. difficile vaccine antigens formulated with Al(OH)₃, at Month 0, 1, 6. Groups 2, 3 and 4 received 2 doses (Month 0, 2) of vaccine formulated with CpG+Al(OH)₃, with CpG adjuvant included at 0.5, 1.0 or 3.6 mg/mL, respectively, and 1 mg/mL Al(OH)₃. Groups 5 and 6 received 2 doses (Month 0, 2) of vaccine formulated with CpG alone, either at 0.5 or 3.6 mg/mL CpG, respectively.

TABLE 12
NHP study design with CpG/Al(OH)₃ or CpG alone adjuvanted formulations

| | Number NHPs | | | Dose | Dosing |
|---|---|---|---|---|---|
| Group | per group | Formulation | Composition (per dose) | Route | Schedule |
| 1 | 5 | Al(OH)₃ | 10 μg of each Txd A&B | 0.5 mL | Month |
| | | | 0.5 mg Al(OH)₃ | IM | 0, 1, 6 |
| 2 | 5 | CpG/Al(OH)₃ | 10 μg of each Txd A&B | 0.5 mL | Month |
| | | | 0.25 mg CpG | IM | 0, 2 |
| | | | 0.5 mg Al(OH)₃ | | |
| 3 | 5 | CpG/Al(OH)₃ | 10 μg of each Txd A&B | 0.5 mL | Month |
| | | | 0.5 mg CpG | IM | 0, 2 |
| | | | 0.5 mg Al(OH)₃ | | |
| 4 | 5 | CpG/Al(OH)₃ | 10 μg of each Txd A&B | 0.5 mL | Month |
| | | | 1.8 mg CpG | IM | 0, 2 |
| | | | 0.5 mg Al(OH)₃ | | |
| 5 | 4 | CpG Alone | 10 μg of each Txd A&B | 0.5 mL | Month |
| | | | 0.25 mg CpG | IM | 0, 2 |
| 6 | 5 | CpG Alone | 10 μg of each Txd A&B | 0.5 mL | Month |
| | | | 1.8 mg CpG | IM | 0, 2 |

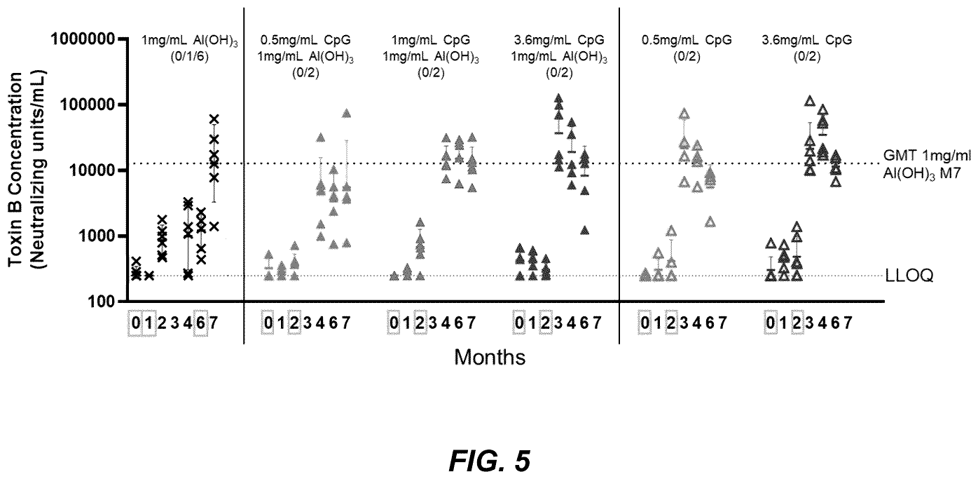

Sera were collected at multiple time points and the ability to neutralize toxin cytotoxic activity was measured in TNAs. The neutralization titers of individual animals for Toxin B are shown in FIG. 5 and GMTs are provided in Tables 13-14. The TNA response to the toxoid antigens formulated with the combination of CpG+Al(OH)₃ revealed a CpG dose response (Groups 2-4). The TNA response measured in sera collected from NHPs immunized with toxoids formulated with 1.0 or 3.6 mg/mL CpG+Al(OH)₃ groups (Groups 3 and 4) were substantially higher than the response measured in sera from the 0.5 mg/mL CpG+1 mg/mL Al(OH)₃ group (Group 2). Toxoid antigens formulated with either level of CpG alone (without of Al(OH)₃) (Groups 5 and 6) also elicited strong TNA responses in NHPs. GMTs for Toxin A are provided in Tables 15-16.

TABLE 13
Toxin B neutralizing titers CpG/Al(OH)₃ adjuvanted formulations

| | Toxin B concentration (Neutralizing units/mL) | | | | | | | |
|---|---|---|---|---|---|---|---|---|
| | | | 0.5 mg/mL CpG | | 1 mg/mL CpG | | 3.6 mg/mL CpG | |
| | Al(OH)₃ | | 1 mg/mL Al(OH)3 | | 1 mg/mL Al(OH)₃ | | 1 mg/mL Al(OH)₃ | |
| | 0/1/6 | | 0/2 | | 0/2 | | 0/2 | |
| Months | GMT | 95% CI | GMT | 95% CI | GMT | 95% CI | GMT | 95% CI |
| 0 | 278 | 225- | 322 | 213- | 250 | 250- | 423 | 266- |
| | | 343 | | 489 | | 250 | | 674 |
| 1 | 250 | 249- | 282 | 242- | 279 | 244- | 395 | 258- |
| | | 251 | | 330 | | 318 | | 605 |
| 2 | 858 | 503- | 347 | 222- | 682 | 354- | 304 | 241- |
| | | 1463 | | 544 | | 1313 | | 384 |
| 3 | Not measured | | Not measured | | Not measured | | Not measured | |
| 4 | 993 | 306- | 4563 | 1261- | 14586 | 8791- | 19012 | 7499- |
| | | 3228 | | 16509 | | 24201 | | 48202 |
| 6 | 1213 | 592- | 3380 | 989- | 15058 | 7104- | 8310 | 2802- |
| | | 2485 | | 11553 | | 31916 | | 24645 |
| 7 | 12770 | 3284- | 5518 | 714- | 12666 | 6889- | 16056 | 4297- |
| | | 49655 | | 42664 | | 23290 | | 59996 |

TABLE 14
Toxin B neutralizing titers for CpG alone adjuvanted formulations
Toxin B concentration (Neutralizing units/mL)

| 0.5 mg/mL CpG | 3.6 mg/mL CpG |
|---|---|
| 0/2 | 0/2 |

| Months | GMT | 95% CI | GMT | 95% CI |
|---|---|---|---|---|
| 0 | 254 | 240-270 | 302 | 185-494 |
| 1 | 304 | 162-571 | 441 | 296-656 |
| 2 | 417 | 127-1370 | 485 | 229-1030 |

| | | |
|---|---|---|
| 3 | Not measured | Not measured |

| | | | | |
|---|---|---|---|---|
| 4 | 13104 | 4908-34989 | 34763 | 16982-71159 |
| 6 | 5466 | 1540-19394 | 11362 | 8045-16046 |
| 7 | 5104 | 2656-9811 | 10027 | 6968-14430 |

TABLE 15
Toxin A neutralizing titers CpG/Al(OH)₃ adjuvanted formulations

| | Toxin A concentration (Neutralizing units/mL) | | | | | | | |
|---|---|---|---|---|---|---|---|---|
| | | | 0.5 mg/mL CpG | | 1 mg/mL CpG | | 3.6 mg/mL CpG | |
| | Al(OH)₃ | | 1 mg/mL Al(OH)3 | | 1 mg/mL Al(OH)₃ | | 1 mg/mL Al(OH)₃ | |
| | 0/1/6 | | 0/2 | | 0/2 | | 0/2 | |
| Months | GMT | 95% CI | GMT | 95% CI | GMT | 95% CI | GMT | 95% CI |
| 0 | 416 | 391- | 377 | 259- | 307 | 211- | 447 | 415- |
| | | 443 | | 547 | | 447 | | 482 |
| 1 | 217 | 134- | 448 | 239- | 453 | 293- | 818 | 494- |
| | | 349 | | 838 | | 701 | | 1355 |
| 2 | 706 | 550- | 491 | 287- | 695 | 580- | 566 | 303- |
| | | 905 | | 841 | | 833 | | 1058 |
| 3 | Not measured | | Not measured | | Not measured | | Not measured | |
| 4 | 353 | 227- | 1520 | 790- | 1896 | 1544- | 2292 | 1502- |
| | | 548 | | 2926 | | 2328 | | 3497 |
| 6 | 297 | 211- | 719 | 206- | 1136 | 762- | 1634 | 852- |
| | | 419 | | 2510 | | 1693 | | 3133 |
| 7 | 1233 | 530- | 420 | 229- | 891 | 566- | 2750 | 1225- |
| | | 2870 | | 771 | | 1405 | | 6174 |

TABLE 16
Toxin A neutralizing titers for CpG alone adjuvanted formulations
Toxin A concentration (Neutralizing units/mL)

| 0.5 mg/mL CpG | 3.6 mg/mL Cp |
|---|---|
| 0/2 | 0/2 |

| Months | GMT | 95% CI | GMT | 95% CI |
|---|---|---|---|---|
| 0 | 441 | 422-460 | 417 | 350-498 |
| 1 | 462 | 237-899 | 617 | 464-821 |
| 2 | 214 | 164-279 | 228 | 137-377 |

| | | |
|---|---|---|
| 3 | Not measured | Not measured |

| | | | | |
|---|---|---|---|---|
| 4 | 841 | 492-1435 | 1659 | 729-3777 |
| 6 | 561 | 233-1347 | 423 | 215-835 |
| 7 | 404 | 202-807 | 640 | 394-1037 |

### Example 5—Immunogenicity of C. difficile Vaccine Antigens Formulated with Different Saponin Containing Liposomal Adjuvants

The relative immunogenicity of C. difficile toxoid antigens formulated with aluminum hydroxide (Al(OH)₃) and different saponin containing liposomal adjuvant (LiNA-2) formulations (e.g. homogeneous and heterogeneous) was compared in rats. Homogeneous and heterogeneous LiNA-2 adjuvants are described herein and in Table 17. Final rat LiNA-2 adjuvant doses were prepared by diluting 1× concentration of LiNA-2 at a 1:5 dilution using PBS buffer at pH 6.2.

TABLE 17
LiNA-2 adjuvant formulation

| | LiNA-2 homogenous | LiNA-2 heterogenous |
|---|---|---|
| Components | (1X, mg/mL) | (1X, mg/mL) |
| 3D-PHAD ® | 0.4 | 0.4 |
| QS-21 | 0.2 | 0.2 |
| DMPC | 14 | 14 |
| DMPG | 1.58 | 1.58 |
| Cholesterol | 10.82 | 10.82 |

All in 10 mM Phosphate, 150 mM NaCl solution at pH 6.2

The toxin neutralization assay described in Example 1 was used to measure the functional cytotoxic activity of sera at multiple time points following immunization.

Wistar Han rats (10 per Group, 8-10 weeks old, Charles River Laboratories) were immunized IM according to the study design in Table 18. Group 1 received C. difficile vaccine antigens formulated with Al(OH)₃. Groups 2 and 3 received the C. difficile vaccine antigens formulated with the homogeneous and heterogeneous LUNA-2 adjuvant, respectively.

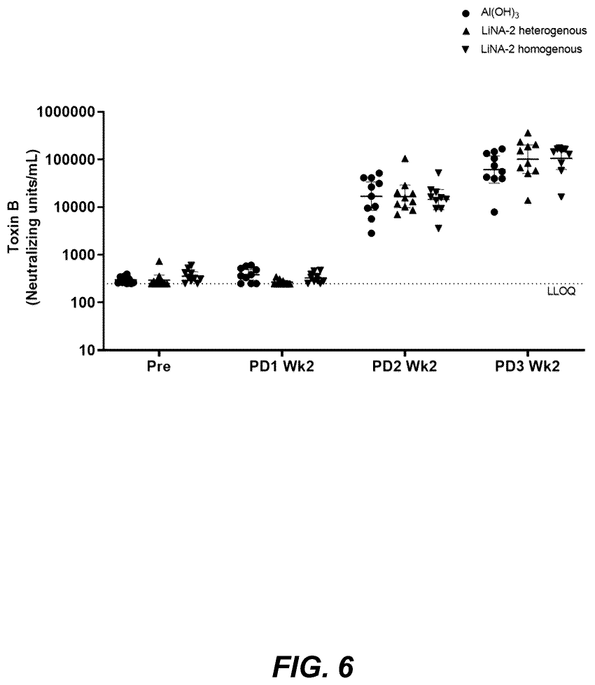

Sera were collected at multiple time points and the ability to neutralize toxin cytotoxic activity was measured in TNAs. Neutralization titers in sera from individual animals for Toxin B are shown in FIG. 6 and illustrate that toxoids formulated with homogeneous and heterogeneous LUNA-2 elicited similar immune responses able to neutralize Toxin B cytotoxicity.

TABLE 18
Rat study design with LiNA-2 adjuvanted formulations

| | | | Dose | | |
|---|---|---|---|---|---|
| | | Composition | Vol | Dosing | Assay |
| Group | Formulation | (per dose, mg/mL) | Route | Schedule | Schedule |
| 1 | Al(OH)₃ | 0.04 mg of each Txd A&B | 250 μl | Week | Week |
| | | 0.4 mg Al(OH)₃ | IM | 0, 4, 8 | 0, 2, 6, 10 |
| 2 | LiNA-2 | 0.04 mg of each Txd A&B | 250 μl | Week | Week |
| | Homogeneous | 0.08 mg 3D-PHAD ® | IM | 0, 4, 8 | 0, 2, 6, 10 |
| | | 0.04 mg QS-21 | | | |
| | | 2.8 mg DMPC | | | |
| | | 0.316 mg DMPG | | | |
| | | 2.16 mg Cholesterol | | | |
| 3 | LiNA-2 | 0.04 mg of each Txd A&B | 250 μl | Week | Week |
| | Heterogeneous | 0.08 mg 3D-PHAD ® | IM | 0, 4, 8 | 0, 2, 6, 10 |
| | | 0.04 mg QS-21 | | | |
| | | 2.8 mg DMPC | | | |
| | | 0.316 mg DMPG | | | |
| | | 2.16 mg Cholesterol | | | |

### Example 6—Determination of Ratio and Concentrations of CpG/Al(OH)₃

Binding studies with various CpG and Al(OH)₃ concentrations and ratios were performed to understand the binding properties of the C. difficile toxoids and CpG 24555 (referred to as “CpG” herein). Formulation parameters affecting the binding of both CpG and the antigens to Al, such as CpG concentration and Al(OH)₃ concentration were investigated. C. difficile drug product for the binding studies was formulated and lyophilized at a concentration of 0.4 mg/mL (200 μg/mL of Toxoid A and 200 μg/mL of Toxoid B) in 10 mM Tris, 4.5% (w/v) trehalose dihydrate, 0.01% (w/v) polysorbate 80, pH 7.4 and reconstituted with the CpG/Al(OH)₃ formulations. The CpG/Al(OH)₃ was formulated as a liquid in 10 mM histidine, 60 mM NaCl, pH 6.5.

UV Spectroscopy method was used to determine the total and bound CpG concentration in CpG/Al(OH)₃ suspension samples. Bound CpG concentration is the amount of CpG associated with Al(OH)₃. Standard optical density at 260 nm was measured to calculate the concentration of oligonucleotides in the sample using Beer-Lambert equation with a theoretical absorptivity constant of CpG (absorptivity constant of CpG at 260 nm is 38.19 mg·L⁻¹·cm). Total oligonucleotide concentration was measured after dissociating CpG from Al(OH)₃. Unbound oligonucleotide concentration was measured by analysis of the supernatant after centrifugation of CpG/Al(OH)₃ suspension to precipitate the particles. The concentration of bound CpG was determined by subtracting the unbound CpG concentration from the total CpG concentration. % bound CpG to Al(OH)₃ is percent bound CpG concentration in total CpG concentration.

To determine % bound toxoid, TxdA and TxdB were separated by anion-exchange chromatography, detected by ultraviolet detection and subsequently quantitated by comparing the peak response of the protein of interest against the response of a reference material of known concentration. Total toxoid concentration was measured after reconstituting a vial of lyophilized C. difficile drug product with saline. Unbound toxoid concentration was measured after reconstituting a vial of lyophilized C. difficile drug product with CpG/Al(OH)₃ by analysis of the supernatant after centrifugation of C. difficile drug product+CpG/Al(OH)₃ suspension to precipitate the particles. The concentration of bound toxoid was determined by subtracting the unbound toxoid concentration from the total toxoid concentration. % bound toxoid to Al(OH)₃ is percent bound toxoid concentration in total toxoid concentration.

Three aluminum levels: 0.85 mg, 1.0 mg and 1.25 mg per 0.5 mL dose were assessed. At each aluminum concentration, different concentrations of CpG were prepared with Al(OH)₃ as shown in Table 19. The prepared CpG/Al(OH)₃ adjuvants were used to reconstitute the lyophilized C. difficile toxoids to reach 400 μg/mL of total toxoids after reconstitution. Table 19 shows full toxoids binding was achieved at a CpG/Al(OH)₃ concentration greater than 1.4/1.7 mg/mL, 1.6/2.0 mg/mL, and 2.0/2.5 mg/mL, respectively.

TABLE 19
% C. difficile binding with varying CpG/Al(OH)3 ratios

| | Reconstituted C. difficile Lyo Drug Product | |
|---|---|---|
| | (TxdA + TxdB at 400 μg/mL) | |
| | % Bound total | % Bound |
| CpG/Al(OH)3 (mg/mL) | Toxoids (A + B)^{a} | CpG |
| At Al(OH)₃ level 1.7 | | |
| 1.2/1.7 | >98% | >99.0% |
| 1.3/1.7 | >98% | >99.1% |
| 1.4/1.7 | >98% | >99.2% |
| 1.5/1.7 | 87% | >99.2% |
| 1.6/1.7 | 77% | >99.3% |
| At Al(OH)₃ level 2.0 | | |
| 1.5/2.0 | >98% | >99.3% |
| 1.6/2.0 | >98% | >99.3% |
| 1.7/2.0 | 94% | >99.4% |
| 1.8/2.0 | 81% | >99.4% |
| 1.9/2.0 | 82% | >99.4% |
| At Al(OH)₃ level 2.5 | | |
| 1.8/2.5 | >98% | >99.4% |
| 1.9/2.5 | >98% | >99.4% |
| 2.0/2.5 | >98% | >99.5% |
| 2.1/2.5 | 96% | >99.5% |
| 2.2/2.5 | 90% | >99.5% |
| 2.4/2.5 | 82% | >99.5% |

^{a}The preliminary LOQ of the assay was 2%

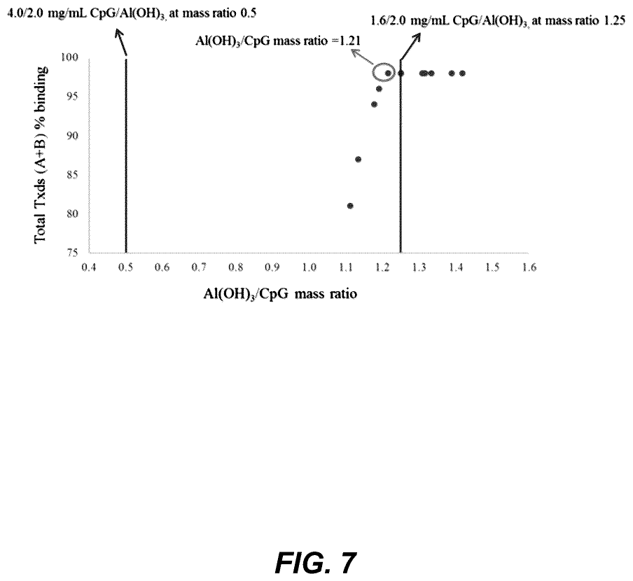

Based on the results in Table 19, the relationship between the % toxoids binding to the mass ratio of the Al(OH)₃/CpG was summarized and plotted in FIG. 7. The toxoids binding to aluminum increased with the increased mass ratio of Al(OH)₃/CpG. Full binding of toxoids to aluminum was achieved when the mass ratio of the Al(OH)₃/CpG was ≥1.21, providing an estimate of the maximum CpG allowed in the formulation with a fixed amount of Al(OH)₃ to achieve complete binding of C. difficile toxoids.

### Example 7—Formulation Development of CpG/Al(OH)₃

Binding and resuspension studies were performed for various CpG/Al(OH)₃ formulations: low dose formulations of 1.0/1.5 mg/mL CpG/Al(OH)₃ and 1.2/1.8 mg/mL CpG/Al(OH)₃, and a high dose formulation of 3.6/1.8 mg/mL CpG/Al(OH)₃, see Tables 20 and 21. C. difficile drug product for the binding studies was formulated and lyophilized at a concentration of 200 μg/mL of Toxoid A and 200 μg/mL of Toxoid B in 10 mM Tris, 4.5% (w/v) trehalose dihydrate, 0.01% (w/v) polysorbate 80, pH 7.4. CpG/Al(OH)₃ adjuvants were formulated as liquids and assessed.

The binding studies were performed as described above. The resuspension properties of formulations were studied by filling each CpG/Al(OH)₃ adjuvant into 1 mL syringes with 0.75 mL fill volume and stoppered with ˜5±1 mm head space. Filled syringes were held horizontally at 5° C. until complete settling was achieved. The resuspension was monitored by counting the average number of shakes from three syringes required to achieve a homogeneous suspension based on visual observation.

Complete CpG binding and C. difficile toxoids binding after reconstitution was observed for various low dose formulations. Further, various formulations took less than five shakes to fully resuspend after initial settling in pre-filled syringes placed in a horizontal orientation, indicating no resuspension concerns. For the high dose formulations various buffer components and NaCl amounts were assessed to improve resuspension and binding.

TABLE 20
CpG/Al(OH)₃ formulations

| CpG/ | | | | CpG | Txds | |
|---|---|---|---|---|---|---|
| Al(OH)₃ | Buffer | NaCl | | Binding | Binding (%) | # Shakes |
| (mg/mL) | (mM) | (mM) | pH | (%) | Total (A + B) | in PFS |
| 1.0/1.5 | 10 | 50 | 6.5 | >98.9 | TxdA: >98 | <5 |
| | Histidine | | | | TxdB: >98 | |
| | | | | | Total Txds 98 | |
| 3.6/1.8 | 10 | 60 | 6.5 | 55.4 | TxdA: >98 | 20 |
| | Histidine | | | | TxdB: 22 | |
| | | | | | Total Txds: 60 | |
| 3.6/1.8 | 10 | 200 | 6.5 | 57.8 | Not tested | 12 |
| | Histidine | | | | | |
| 3.6/1.8 | 25 | 150 | 6.5 | 21.5 | TxdA: 89 | <5 |
| | Phosphate | | | | TxdB: 3 | |
| | | | | | Total Txds: 46 | |
| 3.6/1.8 | 20 | 150 | 6.5 | 24.7 | TxdA: 82 | <5 |
| | Phosphate | | | | TxdB: 6 | |
| | | | | | Total Txds: 44 | |
| 3.6/1.8 | 20 | 60 | 6.5 | 24.1% | TxdA: 97 | <5 |
| | Phosphate | | | | TxdB: 6 | |
| | | | | | Total Txds: 51.5 | |
| 3.6/1.8 | 25 | 80 | 7.0 | 19.0% | TxdA: 87 | <5 |
| | Phosphate | | | | TxdB: 0 | |
| | | | | | Total Txds: 43.5 | |
| 3.6/1.8 | 15 | 40 | 6.0 | 28.4% | TxdA: > 98 | <5 |
| | Phosphate | | | | TxdB: 13 | |
| | | | | | Total Txds: 55.5 | |

TABLE 21
CpG/Al(OH)₃ formulations

| CpG/ | | | | | | |
|---|---|---|---|---|---|---|
| Al(OH)₃ | | | | | | # Shakes |
| Conc. | Buffer | NaCl | | CpG | Txds | Resuspension |
| (mg/mL) | (mM) | (mM) | pH | Binding (%) | Binding (%) | in vials and PFS |
| 1.2/1.8 | 10 | 60 | 6.5 | >99.1 | TxdA: >98 | <5 |
| | Histidine | | | | TxdB: >98 | |
| 1.2/1.8 | 20 | 150 | 6.5 | 66.4 | TxdA: 94 | <5 |
| | Phosphate | | | | TxdB: 9 | |
| 1.2/1.8 | 20 | 60 | 6.5 | 59.7 | TxdA: >98 | <5 |
| | Phosphate | | | | TxdB: 5 | |
| 1.2/1.8 | 10 | 60 | 6.5 | 71.4 | Txd A: >98 | <5 |
| | Phosphate | | | | Txd B: 7 | |

### Example 8—A Phase 1 Randomized and Phase 2 Study to Evaluate the Safety, Tolerability, Immunogenicity, and Immunopersistence of a Clostridioides difficile Vaccine Administered in a 2-Dose Regimen with Novel Adjuvants in Healthy Adults

This Phase 1/2 study in adults ≥50 to <85 years of age evaluates the safety and immunogenicity of various C. difficile vaccine formulations. It assess whether adjuvanted formulations show a similar safety profile with improved immunogenicity (more rapid rise in protective antibodies with maintained durability) in a 2-dose regimen when compared to the current Al(OH)₃-containing vaccine formulation in a 3-dose regimen.

### Phase 1

Phase 1 identifies the preferred adjuvant(s) and dosing schedule. Using a parallel-group design, approximately 140 healthy adults ≥65 to <85 years of age will be randomized equally to 1 of 7 groups (20 participants per group) and will receive the C. difficile vaccine administered with 1 of 3 novel formulations on a 0- and 2-month or 0- and 6-month dosing schedule, or the C. difficile vaccine administered in the current Al(OH)₃-containing formulation (control) on a 0-, 1-, and 6-month dosing schedule. Blood will be collected from all participants at screening and at Visits 1 through 7 for safety laboratory assessments. Additional blood will be collected from all participants at Visits 1 and 3 through 8 for immunogenicity assessments. Participants will be followed for safety through 6 months after the last study injection (Visit 8). The groups will receive one of the following C. difficile vaccine: investigational C. difficile vaccine+low-dose CpG+Al(OH)₃ at 0-2 Months or 0-6 Month; investigational C. difficile vaccine+high-dose CpG only at 0-2 Months or 0-6 Months; investigational C. difficile vaccine+LiNA-2 at 0-2 Months or 0-6 Months; or investigational C. difficile vaccine+Al(OH)₃ only at 0-1-6 Months (current formulation; control)

### Phase 2

Based on safety and immunogenicity data collected during Phase 1, 1 or 2 adjuvant(s) and the corresponding dosing schedule will be selected to proceed into Phase 2. If 1 adjuvant is selected, Phase 2 will enroll a total of approximately 215 healthy adults, with approximately 50 participants ≥50 to <65 years of age and approximately 165 participants ≥65 to <85 years of age. All participants will receive 2 doses of the adjuvanted C. difficile vaccine on the same dosing schedule (0- and 2-month or 0- and 6-month) in an open-label manner. In the event that 2 adjuvant formulations are selected after Phase 1, an additional vaccine group consisting of 215 participants (50 participants ≥50 to <65 years of age and 165 participants ≥65 to <85 years of age) will be added for the second adjuvant formulation, and participants will be randomized equally to 1 of 2 vaccine groups that will receive the C. difficile vaccine formulated with 1 of the 2 selected adjuvants.

Participants will be followed for safety through 6 months after the last vaccination and for antibody persistence for up to 4 years after the last vaccination. Blood will be collected from all participants at Visits 1, 2, and 4 through 9 for immunogenicity assessments.

### Study Arms and Duration:

The study is conducted in 2 phases. Participants in Phase 1 take part in the study for approximately 12 months. Participants in Phase 2 take part in the study for up to approximately 4.5 years (2- to 6-month vaccination period, 6-month safety follow-up period, and up to 3.5 years of antibody persistence follow-up).

### Phase 1 Objectives

### Primary

To describe the safety profile of the C. difficile vaccine when administered in a 2-dose regimen with novel adjuvants in healthy adults
   Endpoints: Local reactions (injection site pain, redness, and swelling); Systemic events (fever, vomiting, diarrhea, headache, fatigue, new or worsening muscle pain, and new or worsening joint pain); adverse events (AEs); serious adverse events (SAEs); medically attended adverse events (MAAEs)
To further describe the safety profile of the C. difficile vaccine when administered in a 2-dose regimen with novel adjuvants in healthy adults
   Endpoints: Hematology and chemistry laboratory parameters

### Secondary

To describe the immune responses elicited by the C. difficile vaccine when administered in a 2-dose regimen with novel adjuvants in healthy adults
   Endpoints: C. difficile TNA (toxin A- and toxin B-specific neutralizing antibody) concentrations
   Estimands: In evaluable participants: GMCs (geometric mean concentration) at each planned postvaccination time point, and GMFRs (geometric mean fold rise) from before vaccination at each postvaccination time point
To describe the safety profile of the C. difficile vaccine when administered in a 2-dose regimen with novel adjuvants in healthy adults
   Endpoints: SAEs and medically attended adverse events from 6 month through 12 months after the last dose

### Phase 2 Objectives

### Primary

To describe the safety profile of the C. difficile vaccine when administered in the selected 2-dose regimen with the selected novel adjuvant(s) in healthy adults
   Endpoints: Local reactions (injection site pain, redness, and swelling); Systemic events (fever, vomiting, diarrhea, headache, fatigue, new or worsening muscle pain, and new or worsening joint pain); adverse events (AEs); serious adverse events (SAEs); medically attended adverse events (MAAEs)
To describe the immune response to the C. difficile vaccine when administered in the selected 2-dose regimen with the selected novel adjuvant(s) in healthy adults
   Endpoints: C. difficile TNA concentrations
   Estimands: In evaluable participants: GMCs at 1 month after the last dose; GMFRs from before vaccination to 1 month after the last dose

### Secondary

To further describe the immune response to the C. difficile vaccine when administered in the selected 2-dose regimen with the selected novel adjuvant(s) in healthy adults
   Endpoints: C. difficile TNA concentrations
   Estimands: In evaluable participants: GMCs at 1 month after the first dose and 6 months after the last dose; GMFRs from before vaccination to 1 month after the first dose and 6 months after the last dose; the proportion of participants with a ≥4-fold rise in TNA concentrations from before vaccination to 1 month after the last dose
To describe the persistence of the immune response to the C. difficile vaccine when administered in the selected 2-dose regimen with the selected novel adjuvant(s) in healthy adults
   Endpoints: C. difficile TNA concentrations
   Estimands: In evaluable participants: GMCs at each planned persistence time point (1, 2, 3, and 4 years after the last dose); GMFRs from before vaccination to each planned persistence time point (from before vaccination to 1, 2, 3, and 4 years after the last dose)
To describe the safety profile of the C. difficile vaccine when administered in a 2-dose regimen with novel adjuvants in healthy adults
   Endpoints: SAEs and medically attended adverse events from 6 month through 12 months after the last dose

1. A immunogenic composition comprising a Clostridioides difficile (C. difficile) toxoid A and/or toxoid B, and a CpG adjuvant comprising at least one CpG or a saponin containing liposomal adjuvant.

2. (canceled)

3. The immunogenic composition of claim 1, wherein the CpG adjuvant comprises a B class CpG.

4. The immunogenic composition of claim 1, wherein the CpG adjuvant comprises CpG 24555 (SEQ ID NO: 48).

5. The immunogenic composition of claim 1, comprising about 0.1 to 5 mg/mL or higher, about 0.1 to about 1.0 mg/mL, about 0.5 to about 1.5 mg/mL, about 0.8 to about 1.8 mg/mL, or about 3.0 to about 4.0 mg/mL CpG.

6. The immunogenic composition of claim 1, comprising about 0.5 mg/mL, about 1.0 mg/mL, about 1.2 mg/mL, or about 3.6 mg/mL CpG.

7. The immunogenic composition of claim 1, comprising about 3.6 mg/mL CpG.

8. The immunogenic composition of claim 1, wherein the CpG adjuvant further comprises an additional adjuvant.

9. The immunogenic composition of claim 1, wherein the CpG adjuvant further comprises an aluminum salt selected from aluminum phosphate, aluminum sulfate and aluminum hydroxide (Al(OH)₃).

10. (canceled)

11. The immunogenic composition of claim 1, comprising a CpG and about 0.1 to 5 mg/mL, about 0.5 to about 1.5 mg/mL, or about 1.0 to about 2.5 mg/mL Al(OH)₃.

12. The immunogenic composition of claim 1, comprising a CpG and about 1.0, about 1.5, about 1.7, about 1.8, about 2.0, or about 2.5 Al(OH)₃.

13. The immunogenic composition of claim 1, comprising about 0.5 to about 3.6 mg/mL CpG and about 0.1 to 5 mg/mL Al(OH)₃.

14. The immunogenic composition of claim 1, comprising:
about 1.0 mg/mL CpG and about 1.5 mg/mL Al(OH)₃;
about 1.2, 1.3, 1.4, 1.5, or 1.6 mg/mL CpG and about 1.7 mg/mL Al(OH)₃;
about 1.5, 1.6, 1.7, 1.8, or 1.9 mg/mL CpG and about 2.0 mg/mL Al(OH)₃;
about 1.8, 1.9, 2.0, 2.1, 2.2, or 2.4 mg/mL CpG and about 2.5 mg/mL Al(OH)₃;
about 3.6 mg/mL CpG and about 1.8 mg/mL Al(OH)₃;
about 1.4 mg/mL CpG and about 1.7 mg/mL or higher Al(OH)₃;
about 1.6 mg/mL CpG and about 2.0 mg/mL or higher Al(OH)₃; or
about 2.0 mg/mL CpG and about 2.5 mg/mL or higher Al(OH)₃.

15. The immunogenic composition of claim 1,
comprising a mass ratio of CpG/Al(OH)₃ of about 1.21 or higher.

16. The immunogenic composition of claim 1, wherein the CpG adjuvant comprises a histidine or phosphate buffer.

17. The immunogenic composition of claim 16, wherein the histidine or phosphate buffer is at a concentration of about 5 mM to 15 mM, or preferably about 10 mM.

18. The immunogenic composition of claim 1, wherein the CpG adjuvant comprises sodium chloride.

19. The immunogenic composition of claim 18, wherein the sodium chloride is at a concentration of about 20 mM to 100 mM, or preferably about 50 mM or about 60 mM.

20. The immunogenic composition of claim 1, wherein the CpG adjuvant has a pH of about 6.0 to 7.0, or preferably about 6.5.

21. The immunogenic composition of claim 1, wherein the CpG adjuvant comprises about 3.6 mg/mL CpG 24555.

22. The immunogenic composition of claim 1, wherein the CpG adjuvant comprises about 1.0 mg/mL CpG 24555 and about 1.5 mg/mL of Al(OH)₃.

23. The immunogenic composition of claim 1, wherein the saponin containing liposomal adjuvant comprises a saponin and a monophosphoryl lipid A (MPLA)-containing liposome composition, wherein the liposome composition comprises i) a lipid bilayer comprising phospholipids and ii) cholesterol.

24. The immunogenic composition of claim 23, wherein the saponin is selected from QS-7, QS-18, QS-21, or a mixture thereof.

25. (canceled)

26. The immunogenic composition of claim 23, wherein the phospholipid is selected from dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG).

27. (canceled)

28. The immunogenic composition of claim 1, wherein the saponin containing liposomal adjuvant comprises QS-21, Monophosphoryl Lipid A (MPLA), DMPC, DMPG and cholesterol.

29. (canceled)

30. The immunogenic composition of claim 1, comprising:
about 0.1 to about 0.4 mg/mL of QS-21, preferably about 0.2 mg/mL of QS-21;
about 0.2 to about 0.6 mg/mL of MPLA, preferably about 0.4 mg/mL of MPLA;
about 5 to about 15 mg/mL of cholesterol, preferably about 11 mg/mL of cholesterol;
about 5 to about 20 mg/mL of DMPC, preferably about 14 mg/mL of DMPC; and/or
about 1.0 to about 2.0 mg/mL of DMPG, preferably about 1.6 mg/mL of DMPG.

31. The immunogenic composition of claim 1, wherein the saponin containing liposomal adjuvant comprises a histidine or phosphate buffer.

32. The immunogenic composition of claim 31, wherein the phosphate buffer is at a concentration of about 5 mM to 15 mM, or preferably about 10 mM, and optionally, further comprises sodium chloride at a concentration of about 100 mM to 200 mM, or preferably about 150 mM.

33. (canceled)

34. The immunogenic composition of claim 1, wherein the saponin containing liposomal adjuvant has a pH of about 5.5 to 7.0, preferably about 6.2.

35. The immunogenic composition of claim 1, wherein the C. difficile toxoid A comprises the amino acid sequence of SEQ ID NO: 4, wherein the initial methionine is absent, and C. difficile toxoid B comprises the amino acid sequence of SEQ ID NO: 6, wherein the initial methionine is absent.

36. The immunogenic composition of claim 1, wherein the C. difficile toxoid A comprises the amino acid sequence of SEQ ID NO: 84, and C. difficile toxoid B comprises the amino acid sequence of SEQ ID NO: 86.

37. The immunogenic composition of claim 1, wherein the composition comprises the C. difficile toxoid A and the C. difficile toxoid B in a ratio of about 3:1 to about 1:1 and/or the composition comprises 200 ρq of toxoid per dose.

38. (canceled)

39. The immunogenic composition of claim 1, wherein the C. difficile toxoid A and toxoid B are lyophilized and reconstituted with a CpG adjuvant or a saponin containing liposomal adjuvant.

40. The immunogenic composition of claim 39, wherein the lyophilized C. difficile toxoid A and toxoid B are reconstituted with:
a CpG adjuvant comprising about 3.6 mg/mL CpG 24555,
a CpG adjuvant comprising about 1.0 mg/mL CpG 24555 and about 1.5 mq/mL Al(OH)₃; and/or
a saponin containing liposomal adjuvant comprising about 0.2 mq/mL QS-21, about 0.4 mg/mL Monophosphoryl 3-Deacyl Lipid A, about 14 mq/mL DMPC, about 1.6 mq/mL DMPG and about 11 mq/mL cholesterol.

41. (canceled)

42. (canceled)

43. The immunogenic composition of claim 1, wherein the composition is administered at a dose volume of 0.5 mL.

44. (canceled)

45. (canceled)

46. A method of eliciting an immune response in a human subject against C. difficile, the method comprising administering to the human subject an immunogenic composition of claim 1.

47. A method of preventing, treating or ameliorating an infection, disease or condition associated with C. difficile in a human subject, the method comprising administering to the human subject an immunogenic composition of claim 1.

48. A method of preventing, treating or ameliorating a medically attended C. difficile infection in a human subject, the method comprising administering to the human subject an immunogenic composition of claim 1.

49. The method of claim 46, wherein the immunogenic composition is administered in a first and second dose.

50. The method of claim 49, wherein the second dose is administered about 2 months after the first dose (e.g. M 0, 2) or about 6 months after the first dose (M 0, 6).

51. The method of claim 46, wherein the human subject is 18 years of age or older, 50 years of age or older, 55 years of age or older, 60 years of age or older, 65 years of age or older, or 70 years of age or older.

52. The method of claim 46, wherein
the immune response elicits neutralizing antibodies against C. difficile toxin A and/or neutralizing antibodies against C. difficile toxin B; and/or
the immune response elicits greater neutralizing antibodies against C. difficile toxin A and/or B after administration of 2 doses of the immunogenic composition compared to administration of 3 doses of the investigational C. difficile vaccine.

53. (canceled)
